SEQUENCE LISTING (25 sequences)

SEQ ID NO: 1 - DNA, 11 bp, Artificial Sequence
  Restriction Enzyme Site
gacnnnnngtc

SEQ ID NO: 2 - DNA, 9 bp, Artificial Sequence
  Restriction Enzyme Site
cagnnnctg

SEQ ID NO: 3 - DNA, 13 bp, Artificial Sequence
  Restriction Enzyme Site
nacnnnngtaycn

SEQ ID NO: 4 - DNA, 12 bp, Artificial Sequence
  Restriction Enzyme Site
cgannnnnntgc

SEQ ID NO: 5 - DNA, 11 bp, Artificial Sequence
  Restriction Enzyme Site
gccnnnnnggc

SEQ ID NO: 6 - DNA, 10 bp, Artificial Sequence
  Restriction Enzyme Site
gatnnnnatc

SEQ ID NO: 7 - DNA, 11 bp, Artificial Sequence
  Restriction Enzyme Site
ccnnnnnnngg

SEQ ID NO: 8 - DNA, 11 bp, Artificial Sequence
  Restriction Enzyme Site
gcannnnntgc

SEQ ID NO: 9 - DNA, 12 bp, Artificial Sequence
  Restriction Enzyme Site
ccannnnnntgg

SEQ ID NO: 10 - DNA, 9 bp, Artificial Sequence
  Restriction Enzyme Site
cacnnngtg

SEQ ID NO: 11 - DNA, 12 bp, Artificial Sequence
  Restriction Enzyme Site
gacnnnnnngtc

SEQ ID NO: 12 - DNA, 11 bp, Artificial Sequence
  Restriction Enzyme Site
cctnnnnnagg

SEQ ID NO: 13 - DNA, 10 bp, Artificial Sequence
  Restriction Enzyme Site
gagtcnnnnn

SEQ ID NO: 14 - DNA, 10 bp, Artificial Sequence
  Restriction Enzyme Site
caynnnnrtg

SEQ ID NO: 15 - DNA, 11 bp, Artificial Sequence
  Restriction Enzyme Site
gcnnnnnnngc

SEQ ID NO: 16 - DNA, 11 bp, Artificial Sequence
  Restriction Enzyme Site
ccannnnntgg

SEQ ID NO: 17 - DNA, 10 bp, Artificial Sequence
  Restriction Enzyme Site
gacnnnngtc

SEQ ID NO: 18 - DNA, 13 bp, Artificial Sequence
  Restriction Enzyme Site
ggccnnnnnggcc

SEQ ID NO: 19 - DNA, 15 bp, Artificial Sequence
  Restriction Enzyme Site
ccannnnnnnnntgg

SEQ ID NO: 20 - DNA, 10 bp, Artificial Sequence
  Restriction Enzyme Site
gaannnnttc

SEQ ID NO: 21 - DNA, 10 bp, Artificial Sequence
  Restriction Enzyme Site
gatnnnnatc

SEQ ID NO: 22 - DNA, 11 bp, Artificial Sequence
  Restriction Enzyme Site
ccnnnnnnngg

SEQ ID NO: 23 - DNA, 12 bp, Artificial Sequence
  Restriction Enzyme Site
gacnnnnnngtc

SEQ ID NO: 24 - DNA, 10 bp, Artificial Sequence
  Restriction Enzyme Site
gacnnnngtc

SEQ ID NO: 25 - DNA, 11 bp, Artificial Sequence
  Restriction Enzyme Site
gacnnnnngtc

(19) United States
(12) Patent Application Publication
(10) Pub. No.: US 2004/0197791 A1
(43) Pub. Date: Oct. 7, 2004

(54) Methods of using nick translate libraries for snp analysis

(76) Inventors: Vladimir L. Makarov, Ann Arbor, MI (US); John P. Langmore, Ann Arbor, MI (US)

Correspondence Address: Melissa L Sistrunk, Fulbright & Jaworski, Suite 5100, 1301 McKinney, Houston, TX, 77010-3095, US

(21) Appl. No.: 10/481,488

(22) PCT Filed: Jun. 25, 2002
(86) PCT No.: PCT/US02/20200

Related U.S. Application Data
(60) Provisional application No. 60/302,172, filed on Jun. 29, 2001.

(51) Int. Cl. C12Q 1/68; C12P 19/34

(52) U.S. Cl. 435/6; 435/91.2

(57) ABSTRACT

The present invention is directed to amplification of a single nucleotide polymorphism by utilizing a library of nick translate molecules. The methods are also directed to highly multiplexed amplification of a nucleic acid sequence to facilitate detection of a single nucleotide polymorphism.

This application claims priority to U.S. Provisional Patent Application No. 60/302,172, filed Jun. 29, 2001, which is incorporated by reference herein in its entirety.

### FIELD OF THE INVENTION

The present invention relates generally to molecular biology and single nucleotide polymorphism amplification methods. More specifically, the present invention relates to amplification of single nucleotide polymorphisms (SNP) from a library of nick translate molecules.

### BACKGROUND OF THE INVENTION

Genetic information is critical in the continuation of life processes. Life is substantially informationally based, and its genetic content controls the growth and reproduction of the organism and its elements. The amino acid sequences of polypeptides, which are critical features of all living systems, are encoded by the genetic material of the cell. Further, the properties of these polypeptides, e.g., as enzymes, functional proteins, and structural proteins, are determined by the sequence of amino acids of which they consist. As structure and function are integrally related, many biological functions may be explained by elucidating the underlying structural features which provide those functions, and these structures are determined by the underlying genetic information in the form of polynucleotide sequences. Further, in addition to encoding polypeptides, polynucleotide sequences also can be involved in control and regulation of gene expression. It therefore follows that the determination of the content of this genetic information has achieved significant scientific importance.

As a specific example, diagnosis and treatment of a variety of disorders may often be accomplished through identification and/or manipulation of the genetic material which encodes for specific disease-associated traits. In order to accomplish this, however, one must first identify a correlation between a particular gene and a particular trait. This is generally accomplished by providing a genetic linkage map through which one identifies a set of genetic markers that follow a particular trait. These markers can identify the location of the gene encoding for that trait within the genome, eventually leading to the identification of the gene. Once the gene is identified, methods of treating the disorder that result from that gene, i.e., as a result of overexpression, constitutive expression, mutation, underexpression, etc., can be more easily developed.

Polymorphisms

One class of genetic markers includes variants in the genetic code termed “polymorphisms.” In the course of evolution, the genome of a species can collect a number of variations in individual bases. These single base changes are termed single-base polymorphisms. Polymorphisms may also exist as stretches of repeating sequences that vary as to the length of the repeat from individual to individual. Where these variations are recurring, e.g., exist in a significant percentage of a population, they can be readily used as markers linked to genes involved in mono- and polygenic traits. In the human genome, single-base polymorphisms occur approximately once per 300 bp. Accordingly, in a human genome of approximately 3 billion bp, one would expect to find approximately 10 million of these polymorphisms.

The use of polymorphisms as genetic linkage markers is thus of critical importance in locating, identifying and characterizing the genes which are responsible for specific traits. In particular, such mapping techniques allow for the identification of genes responsible for a variety of disease or disorder-related traits which may be used in the diagnosis and or eventual treatment of those disorders. Given the size of the human genome, as well as those of other mammals, it is desirable to provide methods of rapidly identifying and screening for polymorphic genetic markers.

Many genetic diseases and traits (i.e. hemophilia, sickle-cell anemia, cystic fibrosis, etc.) reflect the consequences of mutations that have arisen in the genomes of some members of a species through mutation or evolution (Gusella, 1986). In some cases, such polymorphisms are only linked to a genetic locus responsible for the disease or trait; in other cases, the polymorphisms are the determinative characteristic of the condition. The ability to detect variations in nucleic acid sequences is of great importance in the field of medical genetics: the detection of genetic variation is essential, inter alia, for identifying polymorphisms for genetic studies, to determine the molecular basis of inherited diseases, to provide carrier and prenatal diagnosis for genetic counseling and to facilitate individualized medicine. Detection and analysis of genetic variation at the DNA level has been performed by karyotyping, analysis of restriction fragment length polymorphisms (RFLPs) or variable nucleotide type polymorphisms (VNTRs), and more recently, analysis of single nucleotide polymorphisms (SNPs) (see, e.g., Lai et al., 1998; Gu et al., 1998; Taillon-Miller et al., 1998; Weiss, 1998; Zhao et al., 1998).

Because single nucleotide polymorphisms constitute sites of variation flanked by regions of invariant sequence, their analysis requires no more than the determination of the identity of the single nucleotide present at the site of variation; it is unnecessary to determine the complete sequence of a gene for each patient.

Identification and Analysis of Polymorphisms

A wide variety of techniques have been developed for SNP detection and analysis, see, e.g., U.S. Pat. No. 5,858,659; U.S. Pat. No. 5,633,134; U.S. Pat. No. 5,719,028; WO98/30717; WO97/10366; WO98/44157; WO98/20165; WO95/12607 and WO98/30883. In addition, ligase based methods are described by WO97/31256 and Chen et al., 1998; mass-spectroscopy-based methods in WO98/12355, WO98/14616 and Ross et al., 1997; PCR-based methods by Hauser et al. (1998); exonuclease-based methods in U.S. Pat. No. 4,656,127; dideoxynucleotide-based methods in WO91/02087; Genetic Bit Analysis or GBA™ in WO92/15712; Oligonucleotide Ligation Assays or OLAs by Landegren et al., (1988) and Nickerson et al. (1990); and primer-guided nucleotide incorporation procedures by Prezant et al. (1992); Ugozzoli et al. (1992); Nyreen et al., (1993).

The methods and arrays of the present invention find use in the amplification and detection of polymorphisms which are present in an individual to facilitate identification of polymorphisms associated with disease. The present invention in a particular embodiment relates to the amplification and detection of specific variants of previously identified polymorphisms.

An assortment of methods have been used to screen for mutations in genes, including polymorphism associated with disease. Often, such methods begin with amplification of individual exons by polymerase chain reaction or amplification of the transcript by reverse transcription polymerase chain reaction. These methods include direct DNA sequencing, allele-specific probes, allele-specific primers and probe arrays.

Repeated sequencing of genomic material from large numbers of individuals, although extremely time consuming, can be used to identify such polymorphisms. Alternatively, ligation methods may be used where a probe having an overhang of defined sequence is ligated to a target nucleotide sequence derived from a number of individuals. Differences in the ability of the probe to ligate to the target can reflect polymorphisms within the sequence. Similarly, restriction patterns generated from treating a target nucleic acid with a prescribed restriction enzyme or set of restriction enzymes can be used to identify polymorphisms. Specifically, a polymorphism may result in the presence of a restriction site in one variant but not in another. This yields a difference in restriction patterns for the two variants, and thereby identifies a polymorphism.

Screening polymorphisms in samples of genomic material may be carried out using arrays of oligonucleotide probes. These arrays may generally be “tiled” for a large number of specific polymorphisms. By “tiling” is generally meant the synthesis of a defined set of oligonucleotide probes which is made up of a sequence complementary to the specific sequence of interest, or preferably to a sample probe comprising a specific sequence of interest which includes a specific polymorphism. Tiling strategies are discussed in detail in Published PCT Application No. WO 95/11995 (U.S. Ser. No. 08/143,312 (Oct. 26, 1993); U.S. Ser. No. 08/284,064 (Aug. 2, 1994)), incorporated herein by reference in its entirety for all purposes.

In particular, nucleic acid-based analyses often require sequence identification and/or analysis, such as in vitro diagnostic assays and methods development, high throughput screening of natural products for biological activity, and rapid screening of perishable items such as donated blood, tissues, or food products for a wide array of pathogens. In all of these cases there are fundamental constraints to the analysis, e.g., limited sample, time, or often both. In these fields of use, a balance must be achieved between accuracy, speed, and sensitivity in the context of these constraints. Most existing methodologies are generally not multiplexed. That is, optimization of analysis conditions and interpretation of results are performed in simplified single determination assays. However, this can be problematic if a large number of samples need to be analyzed accurately quickly.

Multiplexing requires additional controls to maintain accuracy. False positive or negative results due to contamination, degradation of sample, presence of inhibitors or cross reactants, and inter/intra strand interactions should be considered when designing the analysis conditions, and these are well known to a skilled artisan.

Available technologies can be used in SNPs analysis. For example, U.S. Pat. No. 5,888,819 describes a technique involving first binding a primer to a single-stranded polynucleotide immediately adjacent a polymorphic site of interest, and extending the primer by a terminating nucleotide such as a labeled ddNTP. Incorporation of the labeled base is then detected indicating what allele is present in the sample at the polymorphic site. A similar technique is described in U.S. Pat. No. 5,302,509. A significant drawback with the single-base extension methods described in U.S. Pat. No. 5,888,819 and U.S. Pat. No. 5,302,509 is that they require labor-intensive affinity or physical separation steps to remove all nonterminating labeled nucleotides prior to detection, so that signal from bound nucleotide can be detected without interference with signal from unbound labeled nucleotides. The complexity of these single-base extension methods renders them impractical for some applications, such as SNPs testing procedures that require rapid testing of large numbers of samples. Thus, there is a significant need for simpler methods of detecting single-base variability in polynucleotides, in particular methods that are capable of detecting incorporated labeled nucleotides in the presence of unbound nucleotides, homogenously, without labor-intensive physical separation steps.

WO 00/55372 is directed to the detection of nucleic acid polymorphisms in luminescence-based assays.

WO 01/32929 regards methods and compositions for SNP analysis, wherein a triplex forming oligonucleotide hybridizes near the SNP and a 3′ to 5′ exonuclease generates a protected nucleic acid tail structure which is then hybridized to a SNP identification probe.

WO 00/66607 is related to detection of a SNP wherein a SNP detection sequence binds downstream from a primer to a target DNA in the direction of a primer extension reaction. The SNP detection sequence has a nucleotide complementary to the SNP and adjacent nucleotides complementary to adjacent nucleotides in the target and an electrophoretic tag bonded to the 5′ nucleotide. The pair of sequences is combined with the target DNA under primer extension conditions, wherein the polymerase has 5′ to 3′ exonuclease activity. When the SNP is present, the electrophoretic tag is released and can be detected by electrophoresis as indicative of the presence of the SNP in the target DNA.

Marino (1996) describes low-stringency-sequence specific PCR (LSSP-PCR). A PCR amplified sequence is subjected to single primer amplification under conditions of low stringency to produce a range of different length amplicons. Different patterns are obtained when there are differences in sequence. The patterns are unique to an individual and of possible value for identity testing.

Single strand conformational polymorphism (SSCP) yields similar results. In this method the PCR amplified DNA is denatured and sequence dependent conformations of the single strands are detected by their differing rates of migration during gel electrophoresis. As with LSSP-PCR above, different patterns are obtained that signal differences in sequence. However, neither LSSP-PCR or SSCP gives specific sequence information and both depend on the questionable assumption that any base that is changed in a sequence will give rise to a conformational change that can be detected. Pastinen (1996) amplifies the target DNA and immobilizes the amplicons. Multiple primers are then allowed to hybridize to site 3′ and contiguous to an SNP site of interest. Each primer has a different size that serves as a code. The hybridized primers are extended by one base using a fluorscently labeled dideoxynucleotide triphosphate. The size of each of the fluorescent products that is produced, determined by gel electrophoresis, indicates the sequence and, thus, the location of the SNP. The identity of the base at the SNP site is defined by the triphosphate that is used. A similar approach is taken by Haff (1997), except that the sizing is carried out by mass spectroscopy and thus avoids the need for a label. However, both methods have the serious limitation that screening for a large number of sites will require large, very pure primers that can have troublesome secondary structures and be very expensive to synthesize.

Hacia (1996) uses a high density array of oligonucleotides and the binding patterns produced from different individuals were compared. The method is attractive in that SNPs can be directly identified but the cost of the arrays is high.

Fan (1997) has reported results of a large scale screening of human sequence-tagged sites. The accuracy of single nucleotide polymorphism screening was determined by conventional ABI resequencing.

Allele specific oligonucleotide hybridization along with mass spectroscopy has been discussed by Ross (1997).

Holland et al. (1991) describes use of DNA polymerase 5′-3′ exonuclease activity for detection of PCT products.

Probe-Based Hybridization Assays

Recently, probe hybridization assays have been performed in array formats on solid surfaces, also called “chip formats.” A large number of hybridization reactions using very small amounts of sample can be conducted using these chip formats, thereby facilitating information rich analyses utilizing reasonable sample volumes.

Various strategies have been implemented to enhance the accuracy of these probe-based hybridization assays. One strategy deals with the problems of maintaining selectivity with assays that have many nucleic acid probes with varying GC content. Stringency conditions used to eliminate single base mismatched cross reactants to GC rich probes will strip AT rich probes of their perfect match. Strategies to combat this problem range from using electrical fields at individually addressable probe sites for stringency control to providing separate micro-volume reaction chambers so that separate wash conditions can be maintained. This latter example would be analogous to a miniaturized microplate. Other systems use enzymes as “proof readers” to allow for discrimination against mismatches while using less stringent conditions.

Although the above discussion addresses the problem of mismatches, nucleic acid hybridization is subject to other errors as well. False negatives pose a significant problem and are often caused by the following conditions:

1) Unavailability of the binding domain often caused by intra-strand folding in the target or probe molecule, protein binding, cross reactant DNA/RNA competitive binding, or degradation of target molecule.

2) Non-amplification of target molecule due to the presence of small molecule inhibitors, degradation of sample, and/or high ionic strength.

3) Problems with labeling systems are often problematic in sandwich assays. Sandwich assays, consisting of labeled probes complementary to secondary sites on the bound target molecule, are commonly used in hybridization experiments. These sites are subject to the above mentioned binding domain problems. Enzymatic chemiluminescent systems are subject to inhibitors of the enzyme or substrate and endogenous peroxidases can cause false positives by oxidizing the chemiluminescent substrate.

Methods regarding allele-specific probes for analyzing polymorphisms are described by e.g., Saiki et al., (1986); EP 235,726 (U.S. Pat. No. 836,378 (Mar. 5, 1986); U.S. Pat. No. 943,006 (Dec. 29, 1986)); and WO 89/11548 (U.S. Pat. No. 197,000 (May 20, 1988); U.S. Pat. No. 347,495 (May 4, 1989)). Allele-specific probes are typically used in pairs. One member of the pair shows perfect complementarity to a wildtype allele and the other members to a variant allele. In idealized hybridization conditions to a homozygous target, such a pair shows an essentially binary response. That is, one member of the pair hybridizes and the other does not. An allele-specific primer hybridizes to a site on target DNA overlapping a polymorphism and primes amplification of an allelic form to which the primer exhibits perfect complementarily (Gibbs, 1989). This primer is used in conjunction with a second primer which hybridizes at a distal site. Amplification proceeds from the two primers leading to a detectable product signifying the particular allelic form is present. A control is usually performed with a second pair of primers, one of which shows a single base mismatch at the polymorphic site and the other of which exhibits perfect complementarily to a distal site. The single-base mismatch impairs amplification and little, if any, amplification product is generated.

Polymorphisms can also be identified by hybridization to oligonucleotide arrays. An example is described in WO 95/11995, which includes arrays having four probe sets. A first probe set includes overlapping probes spanning a region of interest in a reference sequence. Each probe in the first probe set has an interrogation position that corresponds to a nucleotide in the reference sequence. That is, the interrogation position is aligned with the corresponding nucleotide in the reference sequence when the probe and reference sequence are aligned to maximize complementarily between the two. For each probe in the first set, there are three corresponding probes from three additional probe sets. Thus, there are four probes corresponding to each nucleotide in the reference sequence. The probes from the three additional probe sets are identical to the corresponding probe from the first probe set except at the interrogation position, which occurs in the same position in each of the four corresponding probes from the four probe sets, and is occupied by a different nucleotide in the four probe sets. Such an array is hybridized to a labeled target sequence, which may be the same as the reference sequence, or a variant thereof. The identity of any nucleotide of interest in the target sequence can be determined by comparing the hybridization intensities of the four probes having interrogation positions aligned with that nucleotide. The nucleotide in the target sequence is the complement of the nucleotide occupying the interrogation position of the probe with the highest hybridization intensity.

WO 95/11995 also describes subarrays that are optimized for detection of variant forms of a precharacterized polymorphism. A subarray contains probes designed to be complementary to a second reference sequence, which can be an allelic variant of the first reference sequence. The second group of probes is designed by the same principles as above except that the probes exhibit complementarity to the second reference sequence. The inclusion of a second group can be particularly useful for analyzing short subsequences of the primary reference sequence in which multiple mutations are expected to occur within a short distance commensurate with the length of the probes (i.e., two or more mutations within 9 to 21 bases).

A further strategy for detecting a polymorphism using an array of probes is described in EP 717,113 (U.S. Pat. No. 327,525 (Oct. 21, 1994). In this strategy, an array contains overlapping probes spanning a region of interest in a reference sequence. The array is hybridized to a labeled target sequence, which may be the same as the reference sequence or a variant thereof. If the target sequence is a variant of the reference sequence, probes overlapping the site of variation show reduced hybridization intensity relative to other probes in the array. In arrays in which the probes are arranged in an ordered fashion stepping through the reference sequence (e.g., each successive probe has one fewer 5′ base and one more 3′ base than its predecessor), the loss of hybridization intensity is manifested as a “footprint” of probes approximately centered about the point of variation between the target sequence and reference sequence.

Conventional Technologies and Limitations

U.S. Pat. No. 4,656,127, for example, discusses a method for determining the identity of the nucleotide present at a particular polymorphic site that employs a specialized exonuclease-resistant nucleotide derivative. A primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data. It has the disadvantages of destroying the amplified target sequences, and unmodified primer and of being extremely sensitive to the rate of polymerase incorporation of the specific exonuclease-resistant nucleotide being used.

French Patent 2,650,840 (U.S. Pat. No. 4,420,902 (Dec. 20, 1983)); PCT Appln. No. WO91/02087) discuss a solution-based method for determining the identity of the nucleotide of a polymorphic site. As in the method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

An alternative method, known as Genetic Bit Analysis or GBA™ is described in PCT Appln. No. 92/15712 (U.S. Pat. No. 664,837 (Mar. 5, 1991); U.S. Pat. No. 775,786 (Oct. 11, 1991). This method uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of French Patent 2,650,840; PCT Appln. No. WO91/02087, this method is preferably a heterogeneous phase assay, in which the primer or the target molecule is immobilized to a solid phase. It is thus easier to perform, and more accurate than the method discussed by PCT Appln. No. 92/15712.

An alternative approach, the “Oligonucleotide Ligation Assay” (“OLA”) (Landegren, U. et al. (1988)) has also been described as capable of detecting single nucleotide polymorphisms. The OLA protocol uses two oligonucleotides which are designed to be capable of hybridizing to abutting sequences of a single strand of a target. One of the oligonucleotides is biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson, et al. have described a nucleic acid detection assay that combines attributes of PCR and OLA (Nickerson et al., 1990). In this method, PCR is used to achieve the exponential amplification of target DNA, which is then detected using OLA. In addition to requiring multiple, and separate, processing steps, one problem associated with such combinations is that they inherit all of the problems associated with PCR and OLA.

Recently, several primer-guided nucleotide incorporation procedures for assaying polymorphic sites in DNA have been described (Komher et al., 1989; Sokolov, 1990); Syv anen et al., 1990; Kuppuswamy et al., 1991; Prezant, 1992; Ugozzoli et al., 1992; Nyren, 1993). These methods differ from GBA™. in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syv anen et al., 1993). Such a range of locus-specific signals could be more complex to interpret, especially for heterozygotes, compared to the simple, ternary (2:0, 1:1, or 0:2) class of signals produced by the GBA™ method. In addition, for some loci, incorporation of an incorrect deoxynucleotide can occur even in the presence of the correct dideoxynucleotide (Komher et al., 1989). Such deoxynucleotide misincorporation events may be due to the Kₘ of the DNA polymerase for the mispaired deoxy-substrate being comparable, in some sequence contexts, to the relatively poor Kₘ of even a correctly base paired dideoxy-substrate (Kornberg et al., 1992; Tabor et al., 1989). This effect would contribute to the background noise in the polymorphic site interrogation.

Nucleic Acid Hybridization

Many molecular biology techniques involve carrying out numerous operations on a large number of samples. They are often complex and time consuming, and generally require a high degree of accuracy. Many techniques are limited in their application by a lack of sensitivity, specificity, or reproducibility. For example, problems with sensitivity and specificity have so far limited the practical applications of nucleic acid hybridization.

Nucleic acid hybridization analysis generally involves the detection of a very small numbers of specific target nucleic acids (DNA or RNA) with probes among a large amount of non-target nucleic acids. In order to keep high specificity, hybridization is normally carried out under the most stringent conditions, achieved through various combinations of temperature, salts, detergents, solvents, chaotropic agents, and denaturants.

Multiple sample nucleic acid hybridization analysis has been conducted on a variety of filter and solid support formats (see Beltz et al., 1985). One format, the so-called “dot blot” hybridization, involves the non-covalent attachment of target DNAs to a filter, which are subsequently hybridized with a radioisotope labeled probe(s). “Dot blot” hybridization gained wide-spread use, and many versions were developed (see Anderson and Young, 1985). The “dot blot” hybridization has been further developed for multiple analysis of genomic mutations (Nanibhushan and Rabin, 1987) and for the detection of overlapping clones and the construction of genomic maps (U.S. Pat. No. 5,219,726). Another format, the so-called “sandwich” hybridization, involves attaching oligonucleotide probes covalently to a solid support and using them to capture and detect multiple nucleic acid targets. (Ranki et al., 1983; UK Patent Application GB 2156074A; U.S. Pat. No. 4,563,419; PCT WO 86/03782; U.S. Pat. No. 4,751,177; PCT WO 90/01564; Wallace et al., 1979; and Connor et al., 1983). Multiplex versions of these formats are called “reverse dot blots”.

Using the current nucleic acid hybridization formats and stringency control methods, it remains difficult to detect low copy number (i.e., 1-100,000) nucleic acid targets even with the most sensitive reporter groups (enzyme, fluorophores, radioisotopes, etc.) and associated detection systems (fluorometers, luminometers, photon counters, scintillation counters, etc.).

This difficulty is caused by several underlying problems associated with direct probe hybridization. One problem relates to the stringency control of hybridization reactions. Hybridization reactions are usually carried out under the stringent conditions in order to achieve hybridization specificity. Methods of stringency control involve primarily the optimization of temperature, ionic strength, and denaturants in hybridization and subsequent washing procedures. Unfortunately, the application of these stringency conditions causes a significant decrease in the number of hybridized probe/target complexes for detection.

Another problem relates to the high complexity of DNA in most samples, particularly in human genomic DNA samples. When a sample is composed of an enormous number of sequences which are closely related to the specific target sequence, even the most unique probe sequence has a large number of partial hybridizations with non-target sequences.

A third problem relates to the unfavorable hybridization dynamics between a probe and its specific target. Even under the best conditions, most hybridization reactions are conducted with relatively low concentrations of probes and target molecules. In addition, a probe often has to compete with the complementary strand for the target nucleic acid.

A fourth problem for most present hybridization formats is the high level of non-specific background signal. This is caused by the affinity of DNA probes to almost any material.

These problems, either individually or in combination, lead to a loss of sensitivity and/or specificity for nucleic acid hybridization in the above described formats. This is unfortunate because the detection of low copy number nucleic acid targets is necessary for most nucleic acid-based clinical diagnostic assays.

Because of the difficulty in detecting low copy number nucleic acid targets, the research community relies heavily on the polymerase chain reaction (PCR) for the amplification of target nucleic acid sequences. The enormous number of target nucleic acid sequences produced by the PCR reaction improves the subsequent direct nucleic acid probe techniques, albeit at the cost of a lengthy and cumbersome procedure.

A distinctive exception to the general difficulty in detecting low copy number target nucleic acid with a direct probe is the in situ hybridization technique. This technique allows low copy number unique nucleic acid sequences to be detected in individual cells. In the in situ format, target nucleic acid is naturally confined to the area of a cell (about 20-50 μm²) or a nucleus (about 10 μm²) at a relatively high local concentration. Furthermore, the probe/target hybridization signal is confined to a microscopic and morphologically distinct area; this makes it easier to distinguish a positive signal from artificial or non-specific signals than hybridization on a solid support.

Mimicking the in situ hybridization in some aspects, new techniques are being developed for carrying out multiple sample nucleic acid hybridization analysis on micro-formatted multiplex or matrix devices (e.g., DNA chips) (Barinaga, 1991; Bains, 1992). These methods usually attach specific DNA sequences to very small specific areas of a solid support, such as micro-wells of a DNA chip. These hybridization formats are micro-scale versions of the conventional “reverse dot blot” and “sandwich” hybridization systems.

The micro-formatted hybridization can be used to carry out “sequencing by hybridization” (SBH) (Barinaga, 1991; Bains, 1992). SBH makes use of all possible n-nucleotide oligomers (n-mers) to identify n-mers in an unknown DNA sample, which are subsequently aligned by algorithm analysis to produce the DNA sequence (Yugoslav Patent Application #570/87, 1987; Drmanac et al., 1989; Strezoska et al., 1991; and U.S. Pat. No. 5,202,231).

There are two formats for carrying out SBH. One format involves creating an array of all possible n-mers on a support, which is then hybridized with the target sequence. This is a version of the reverse dot blot. Another format involves attaching the target sequence to a support, which is sequentially probed with all possible n-mers. Both formats have the fundamental problems of direct probe hybridizations and additional difficulties related to multiplex hybridizations. This inability to achieve “sequencing by hybridization” by a direct hybridization method lead to a so-called “format 3”, which incorporates a ligase reaction step. While, providing some degree of improvement, it actually represents a different mechanism involving an enzyme reaction step to identify base differences.

Southern, United Kingdom Patent Application GB 8810400, 1988 (U.S. Pat. No. 6,054,270 (Apr. 25, 2000)); Southern et al. (1992) proposed using the “reverse dot blot” format to analyze or sequence DNA. Southern identified a known single point mutation using PCR amplified genomic DNA. Southern also described a method for synthesizing an array of oligonucleotides on a solid support for SBH. However, Southern did not address how to achieve optimal stringency condition for each oligonucleotide on an array.

Fodor et al. (1993) used an array of 1,024 8-mer oligonucleotides on a solid support to sequence DNA. In this case, the target DNA was a fluorescently labeled single-stranded 12-mer oligonucleotide containing only nucleotides the A and C bases. A concentration of 1 pmol (about 6×10¹¹ molecules) of the 12-mer target sequence was necessary for the hybridization with the 8-mer oligomers on the array. The results showed many mismatches. Like Southern, Fodor et al., did not address the underlying problems of direct probe hybridization, such as stringency control for multiplex hybridizations. These problems, together with the requirement of a large quantity of the simple 12-mer target, indicate severe limitations to this SBH format.

Concurrently, Drmanac et al. (1993) used the above discussed second format to sequence several short (116 bp) DNA sequences. Target DNAs were attached to membrane supports (“dot blot” format). Each filter was sequentially hybridized with 272 labeled 10-mer and 11-mer oligonucleotides. A wide range of stringency conditions were used to achieve specific hybridization for each n-mer probe; washing times varied from 5 minutes to overnight, and temperatures from 0° C. to 16° C. Most probes required 3 hours of washing at 16° C. The filters had to be exposed for 2 to 18 hours in order to detect hybridization signals. The overall false positive hybridization rate was 5% in spite of the simple target sequences, the reduced set of oligomer probes, and the use of the most stringent conditions available.

Fodor et al (1991) used photolithographic techniques to synthesize oligonucleotides on a matrix. Pirrung et al., in U.S. Pat. No. 5,143,854, teach large scale photolithographic solid phase synthesis of polypeptides in an array fashion on silicon substrates.

In another approach of matrix hybridization, Beattie et al. (1992) used a microrobotic system to deposit micro-droplets containing specific DNA sequences into individual microfabricated sample wells on a glass substrate. The hybridization in each sample well is detected by interrogating miniature electrode test fixtures, which surround each individual microwell with an alternating current (AC) electric field.

Regardless of the format, all current micro-scale DNA hybridizations and SBH approaches do not overcome the underlying problems associated with nucleic acid hybridization reactions. They require very high levels of relatively short single-stranded target sequences or PCR-amplified DNA, and produce a high level of false positive hybridization signals even under the most stringent conditions. In the case of multiplex formats using arrays of short oligonucleotide sequences, it is not possible to optimize the stringency condition for each individual sequence with any conventional approach because the arrays or devices used for these formats can not change or adjust the temperature, ionic strength, or denaturants at an individual location, relative to other locations. Therefore, a common stringency condition must be used for all the sequences on the device. This results in a large number of non-specific and partial hybridizations and severely limits the application of the device. The problem becomes more compounded as the number of different sequences on the array increases, and as the length of the sequences decreases below 10-mers or increase above 20-mers. This is particularly troublesome for SBH, which requires a large number of short oligonucleotide probes.

More recently, attempts have been made at microchip based nucleic acid arrays to permit the rapid analysis of genetic information by hybridization. Many of these devices take advantage of the sophisticated silicon manufacturing processes developed by the semiconductor industry over the last forty years. In these devices, many parallel hybridizations may occur simultaneously on immobilized capture probes. Stringency and rate of hybridization is generally controlled by temperature and salt concentration of the solutions and washes. Even though they are of very high probe densities, such a “passive” micro-hybridization approaches have several limitations, particularly for arrays directed at reverse dot blot formats, for base mismatch analysis, and for re-sequencing and sequencing by hybridization applications.

First, as all nucleic acid probes are exposed to the same conditions simultaneously, capture probes must have similar melting temperatures to achieve similar levels of hybrid stringency. This places limitations on the length, GC content and secondary structure of the capture probes. Also, single-stranded target fragments must be selected out for the actual hybridization, and extremely long hybridization and stringency times are required (see, e.g., Guo, Z, et al., Nucleic Acid Research, V.22, #24, pp. 5456-5465, 1994).

Second, for single base mismatch analysis and re-sequencing applications a relatively large number of capture probes (>16) must be present on the array to interrogate each position in a given target sequence. For example, a 400 base-pair target sequence would require an array with over 12,000 different probe sequences (see, e.g., Kozal et al., 1996).

Third, for many applications, large target fragments, including PCR or other amplicons, can not be directly hybridized to the array. Frequently, complicated secondary processing of the amplicons is required, including: (1) further amplification; (2) conversion to single-stranded RNA fragments; (3) size reduction to short oligomers, and (4) intricate molecular biological/enzymatic reactions steps, such as ligation reactions.

Fourth, for passive hybridization the rate is proportional to the initial concentration of the target fragments in the solution, therefore, very high concentrations of target is required to achieve rapid hybridization.

Fifth, because of difficulties controlling hybridization conditions, single base discrimination is generally restricted to capture oligomers sequences of 20 bases or less with centrally placed differences (see, e.g., Chee, 1996; Guo et al., 1994; Kozal et al., 1996).

### SUMMARY OF THE INVENTION

Single nucleotide polymorphisms (SNPs) are important markers for the identification of genomic regions associated with complex diseases in humans. Understanding genetic variations promises to have a great impact on our ability to predict the individual response to therapeutics, reduce cost and time associated with clinical trials, and improve the efficacy of existing and next generation drugs. There are likely over 10 million SNPs in the human genome, and analysis of even 1% of all human variations (100,000 SNPs) would result in a high-resolution whole genome molecular fingerprint that can be used to uniquely identify an individual. Considering that association studies of complex diseases and pharmacogenomics applications would require analysis of many individuals (10²-10³), the total number of polymorphisms to analyze is tremendous and can be only achieved by high throughput parallel analysis of multiple DNA samples. Several genotyping platforms are currently available, but at a current average price of 0.5-1 dollar per genotype, their use in large-scale SNP genotyping studies may be prohibitively expensive.

Genotyping of SNPs requires two steps: DNA amplification and SNP detection. For high throughput analysis of potentially all SNPs from a large number of samples, both the amplification and the detection steps should be highly multiplexed and inexpensive. Whereas there are many new ideas concerning how to perform parallel detection of thousands of DNA variations simultaneously, few address the issue of highly multiplexed sample amplification.

An additional important factor limiting the whole-genome genotyping is the amount of DNA isolated from a standard blood sample. Typically, 1 ml of blood sample gives about 10 μg of DNA. Because 10-50 ng of DNA is necessary for reproducible amplification of SNP containing loci by PCR, the genotype analysis is usually restricted to only 200-1,000 SNPs per sample.

A skilled artisan is cognizant that any method to make an amplifiable nick translate molecule for SNP analysis is within the scope of the present invention. A skilled artisan also recognizes that, in a preferred method, the amplifiable nick translate molecule is generated by methods comprising at least fragmenting a DNA sample; attaching an adaptor to one end of the fragmented molecules, such as by covalent attachment, wherein the adaptor comprises a nick; nick translating with a DNA polymerase having 5′→3′ polymerase activity and 5′→3′ exonuclease activity; and attaching a second adaptor to the other end of the nick translated product. The nick translate molecule may be amplified by primer sequences for the adaptors. Although the nick is preferably generated by an adaptor comprising more than one oligonucleotide, wherein the oligonucleotide assembly has a nick between them, a skilled artisan recognizes that the nick may be generated by any standard means in the art.

The skilled artisan recognizes that, as the present invention is directed to methods and compositions regarding amplification of a SNP and/or high multiplex amplification of a nucleic acid sequence to facilitate SNP detection, standard means in the art are available for the terminal step of detecting the SNP. For example, the SNP may be identified by commonly used microarray analysis techniques, hybridization techniques, fluorescence techniques, etc. In one embodiment, following SNP amplification provided by the teachings described herein, the SNP is detected by a microarray, such as by Affymetrix GeneChip® technology. In relation to this, U.S. Pat. Nos. 5,858,659 and 6,045,996 are directed to such technology. U.S. Pat. No. 5,858,659 provides a method of employing arrays of oligonucleotide probes that are complementary to target nucleic acids which correspond to a marker sequence for an individual. The probes are arranged in detection blocks, each block capable of discriminating the three genotypes for a given marker. U.S. Pat. No. 6,045,996 regards methods for improving the discrimination of hybridization of the target nucleic acids to the probes on the substrate-bound oligonucleotide arrays. In this method of improving a hybridization assay, the array comprising a surface of covalently attached oligonucleotide probes having different known sequences in discrete locations is incubated with a hybridization mixture including betaine. Thus, a skilled artisan recognizes that there are not only multiple methods for actual detection of the SNP following its amplification by the novel methods described herein, but that a variety of improvements exist therein.

The following definitions are provided to assist in understanding the nature of the invention.

The term “down-stream (nick-attaching) adaptor molecules” as used herein refers to partially double-stranded or completely single-stranded DNA molecules that can be linked to 3′ or 5′ DNA termini at a nick within double-stranded DNA molecule. Their design has a minimum of two domains: 1) a domain that facilitates ligation to the 3′ or 5′ DNA termini within the nick or a domain that facilitates priming of the polymerization reaction which results in the extension of the 3′ terminus near the nick; 2) a domain that facilitates amplification. In addition, down-stream adaptors may comprise additional domains that facilitate manipulation of the DNA strand, including, for example, recombination, amplification, detection, affinity capture, and inhibition of self-ligation.

The term “haplotype” as used herein is defined as a combination of two or more separate polymorphisms that are located on the same copy of the chromosome inherited from one parent.

The term “kernel” as used herein is a known sequence of DNA that is used to select the amplified region within the template DNA.

The terms “multiplex” or “multiplexing” as used herein refers to processing multiple DNA sequences at the same time and in the same reactions such that the information from each sequence can be recovered later.

The term “nick translation” as used herein refers to a coupled polymerization/degradation process that is characterized by a coordinated 5′→3′ DNA polymerase activity and a 5′→3′ exonuclease activity.

The term “nick translation initiation site” as used herein is a free 3′OH-containing terminus at a nick or a small gap within an adaptor molecule. Where the nick site is contained within an adaptor, the nick translation initiation site can be: 1) a part of the adaptor before attachment to DNA, 2) created by annealing a priming oligonucleotide to the distal primer binding region of the adaptor before or after the first nick translation reaction, or, 3) created by recombination of two different adaptors.

The term “nick translate molecule” as used herein refers to nucleic acid molecules produced by coordinated 5′→3′ polymerase activity, such as DNA polymerase, and 5′→3′ exonuclease activity. The two activities can be present within on enzyme molecule (such as DNA polymerase I or Taq DNA polymerase). In a preferred embodiment, they have adaptor sequences at their 5′ and 3′ termini.

The term “up-stream (terminus-attaching) adaptor molecules” as used herein are short artificial DNA molecules that are ligated to the ends of DNA fragments. Their design has a minimum of two domains: 1) a domain that facilitates ligation to the ends of template DNA molecules; and 2) a domain that facilitates initiation of a nick-translation reaction. In addition, up-stream adaptors may comprise additional domains that facilitate manipulation of the DNA strand, including, for example, recombination, amplification, detection, affinity capture, and inhibition of self-ligation.

It is an object of the present invention to provide a method of amplifying a single nucleotide polymorphism (SNP) from a DNA sample, comprising obtaining the DNA sample comprising said single nucleotide polymorphism to be amplified; generating at least one nick translate molecule from said DNA sample, wherein said nick translate molecule comprises said single nucleotide polymorphism; and amplifying said nick translate molecule. In a specific embodiment, the step of generating the nick translate molecule comprises attaching upstream adaptor molecules to ends of DNA sample molecules to provide a nick translation initiation site; subjecting the DNA molecules to nick translation comprising DNA polymerization and 5′→3′ exonuclease activity to produce the nick translate molecules; and attaching downstream adaptor molecules to the nick translate molecules to produce adaptor attached nick translate molecules.

In another object of the present invention, there is a method of producing a library of SNP-containing DNA molecules, comprising obtaining a DNA sample comprising at least one SNP; digesting DNA molecules of the DNA sample with a sequence-specific endonuclease; attaching upstream adaptor molecules to ends of DNA molecules of the sample to provide a nick translation initiation site; subjecting the DNA molecules to nick translation comprising DNA polymerization and 5′-3′ exonuclease activity to produce the nick translate molecules, wherein said nick translate molecules comprise said SNP; attaching downstream adaptor molecules to the nick translate molecules to produce adaptor attached nick translate molecules; and separating the SNP-containing nick translate molecules. In a specific embodiment, the separating step is by size. In another specific embodiment, the separating step is by hybridization. In an additional specific embodiment, the separating step further comprises amplification of at least one said SNP-containing nick translate molecules. In an additional specific embodiment, the amplification is by polymerase chain reaction.

In an additional object of the present invention, there is a method of analyzing a SNP from a plurality of DNA samples, comprising obtaining said plurality of DNA samples, wherein at least one DNA sample comprises said SNP; digesting DNA molecules of the DNA sample with a sequence-specific endonuclease; attaching upstream adaptor molecules to ends of DNA molecules of the sample to provide a nick translation initiation site; subjecting the DNA molecules to nick translation comprising DNA polymerization and 5′-3′ exonuclease activity to produce the nick translate molecules; wherein said nick translate molecules comprise said at least one SNP; attaching downstream adaptor molecules to the nick translate molecules to produce adaptor attached nick translate molecules; and separating the SNP-containing nick translate molecules. In a specific embodiment, the upstream adaptors are nonidentical. In an additional specific embodiment, the separating step is by size. In another specific embodiment, the separating step is by hybridization. In a further specific embodiment, the separating step further comprises amplification of said SNP-containing nick translate molecules.

In an additional object of the present invention, there is a method of isolating a specific SNP-containing nick translate molecule from a plurality of nick translate molecules, comprising obtaining a plurality of SNP-containing nick translate molecules; ligating to an end of the SNP-containing nick translate molecules a first oligonucleotide to form a first oligonucleotide-nick translate molecule complex, wherein said first oligonucleotide comprises nucleic acid sequence complementary to an adaptor end of said nick translate molecules; a double stranded region; wherein the double stranded region facilitates the formation of an adjacent hairpin or loop in the oligonucleotide; a free 3′ OH; and a 5′ phosphate; attaching to said first oligonucleotide-nick translate molecule complex a second oligonucleotide to form a first oligonucleotide-nick translate molecule-second oligonucleotide-complex, wherein the second oligonucleotide comprises nucleic acid sequence adjacent to an adaptor end of said nick translate molecules; nucleic acid sequence nonidentical to a restriction endonuclease site used in generating the nick translate molecules; and an affinity tag; isolating the nick translate molecule-first oligonucleotide-second oligonucleotide-complex from said plurality of nick translate molecules by said affinity tag. In a further specific embodiment, the attaching step further comprises ligation of said second oligonucleotide to said first oligonucleotide-nick translate molecule complex. In additional embodiments, the first oligonucleotide further comprises a labile base, the double stranded region of said first oligonucleotide is approximately six to eight bases, the double stranded region of said first oligonucleotide is at least about 4 bases, and/or the double stranded region of said first oligonucleotide is no more than about 100 bases. In an additional specific embodiment, the nucleic acid sequence in said second oligonucleotide which corresponds to the nucleic acid sequence adjacent to an adaptor end of said nick translate molecules is five nucleotides in length. In a specific embodiment, the affinity tag of said second oligonucleotide is biotin.

In another object of the present invention method of isolating a complementary nucleic acid molecule to a specific SNP-containing nick translate molecule, comprising obtaining a plurality of nick translate molecules; introducing to said plurality an oligonucleotide comprising a nucleic acid sequence complementary to a specific region of said specific nick translate molecule; a nucleic acid sequence substantially nonidentical to a sequence in said specific nick translate molecule, wherein the nucleic acid sequence is 5′ to said sequence in i); and an affinity tag, wherein the oligonucleotide hybridizes to the specific nick translate molecule; extending the oligonucleotide by polymerization to form a complementary nucleic acid molecule for the specific nick translate molecule; and isolating the extended complementary nucleic acid sequence molecule from the plurality of nick translate molecules. In a specific embodiment, the method further comprises amplifying said complementary nucleic acid molecule. In another specific embodiment, the amplification step is by polymerase chain reaction. In an additional specific embodiment, the oligonucleotide further comprises a hairpin or loop structure.

In an additional object of the present invention, there is a method of amplifying a nucleic acid sequence for SNP analysis, comprising generating a nick translate molecule comprising the nucleic acid sequence and comprising an upstream adaptor and a downstream adaptor; performing polymerase chain reaction to amplify said nick translate molecule using a first oligonucleotide complementary to an adaptor sequence of said nick translate molecule and a second oligonucleotide complementary to a known nucleic acid sequence of said nick translate molecule. In a further specific embodiment, the step of generating said nick translate molecule comprises attaching said upstream adaptor molecule to ends of DNA molecules comprising said nucleic acid sequence for SNP analysis to provide a nick translation initiation site; subjecting the DNA molecules to nick translation comprising DNA polymerization and 5′-3′ exonuclease activity to produce the nick translate molecules; and attaching downstream adaptor molecules to the nick translate molecules to produce adaptor attached nick translate molecules.

In another object of the present invention there is a method of multiplex amplification of a plurality of nucleic acid sequences for SNP analysis, comprising generating a plurality of nick translate molecules comprising a nucleic acid sequence comprising said SNP, wherein each nick translate molecule comprises a first adaptor and a second adaptor; introducing to said plurality of nick translate molecules a plurality of first oligonucleotides complementary to said first or second adaptor sequence of said nick translate molecules and a plurality of second oligonucleotides, wherein each second oligonucleotide is complementary to a known nucleic acid sequence in a nick translate molecule; and amplifying the region in the nucleic acid sequence of said nick translate molecules between said first oligonucleotide and said second oligonucleotide by polymerase chain reaction.

In another object of the present invention, there is a method of multiplex amplification of a plurality of nucleic acid sequences for SNP analysis, comprising generating a plurality of nick translate molecules each comprising a nucleic acid sequence comprising said SNP, wherein each nick translate molecule comprises a first adaptor and a second adaptor; introducing to said plurality of nick translate molecules a plurality of first oligonucleotides complementary to said first adaptor sequence of said nick translate molecules and a plurality of second oligonucleotides, wherein the second oligonucleotide comprise nucleic acid sequence complementary to said second adaptor; and multiple nucleotide bases at the 3′ terminal end of said second oligonucleotide which are complementary to corresponding multiple nucleotide bases in the nucleic acid sequence of said nick translate molecule immediately adjacent to said second adaptor; amplifying the region in the nucleic acid sequence of said nick translate molecules between said first oligonucleotide and said second oligonucleotide by polymerase chain reaction, whereby the amplification of the nucleic acid sequence occurs only under conditions wherein the second oligonucleotide anneals to said nick translate molecule at said multiple nucleotide bases immediately adjacent to the second adaptor. In a specific embodiment, the multiple nucleotide bases comprise two bases. In a specific embodiment, the multiple nucleotide bases comprise three bases.

In an object of the present invention, there is a method of multiplex amplification of a nucleic acid sequence comprising a SNP of interest, wherein the nucleic acid sequence is adjacent to a known nucleic acid sequence, comprising obtaining a DNA sample; processing said DNA sample to generate a library of nick translate molecules, wherein said nick translate molecules are separated into sublibraries of molecules that are complementary to specified positions within a region of the DNA, and wherein said sublibraries are partitioned into chambers of a solid support; and amplifying by polymerase chain reaction within said chambers at least one nick translate molecule or fragment thereof using a primer from said known nucleic acid sequence. In a specific embodiment, the DNA sample further comprises a genome. In another specific embodiment, the solid support is a microwell plate.

In an additional object of the present invention, there is a method of multiplex amplification of a nucleic acid sequence comprising a SNP of interest, wherein the nucleic acid sequence is adjacent to a known nucleic acid sequence, comprising obtaining a DNA sample; processing said DNA sample to generate a library of nick translate molecules, wherein said nick translate molecules are in a pooled collection and wherein the nick translate molecules are comprised of sequences complementary to unknown positions within a region of the template DNA; and amplifying by polymerase chain reaction within said pooled collection at least one nick translate molecule or fragment thereof using a primer from said known nucleic acid sequence. In a specific embodiment, the pooled collection is in a single tube. In another specific embodiment, the method further comprises applying said amplified nick translate molecules to a DNA microarray, wherein hybridization of a nick translate molecule to the DNA microarray identifies said SNP.

In another object of the present invention, there is a method of assaying a DNA sample for the presence of multiple specific SNPs, comprising generating a plurality of nick translate molecules from said DNA molecules of said sample, wherein said plurality of nick translate molecules comprise said multiple SNPs; introducing to said nick translate molecules a plurality of oligonucleotides, wherein an oligonucleotide hybridizes adjacent to a specific SNP location and wherein the 3′ base of said oligonucleotide is variable; extending by polymerization from said oligonucleotide, whereby extension only occurs if said variable 3′ base of said oligonucleotide is complementary to the corresponding nucleotide of said specific SNP; and detecting said extended oligonucleotide. In a specific embodiment, the detection step further comprises separation by size. In a further specific embodiment, the size detection is by capillary electrophoresis. In an additional specific embodiment, the extended oligonucleotide is detected by detecting a label on the 3′ base of said oligonucleotide. In another specific embodiment, the label is fluorescent. In a further specific embodiment, the multiple specific SNPs are detected concomitantly, and wherein the labels for multiple nonidentical oligonucleotides in said plurality of oligonucleotides are distinguishable.

In an object of the present invention, there is a method of assaying a DNA sample for the presence of multiple specific SNPs, comprising generating a plurality of nick translate molecules from said DNA molecules of said sample, wherein said plurality of nick translate molecules comprise said SNP; introducing to said nick translate molecules a plurality of first oligonucleotides, wherein a first oligonucleotide hybridizes such that its 5′ end is adjacent to a specific SNP; extending said first oligonucleotide by primer extension to form a plurality of nick translate molecule-first oligonucleotide extension product hybrids; introducing to said plurality of hybrids a plurality of second oligonucleotides, wherein a second oligonucleotide hybridizes adjacent to the specific SNP and comprises a variable nucleotide 3′ end; and ligating the 3′ end of said second oligonucleotide to the 5′ end of said first oligonucleotide extension product, whereby said ligation occurs only if said variable nucleotide is complementary to said SNP, to form a ligated molecule of said second oligonucleotide and said first oligonucleotide extension product; and detecting said ligated molecule. In a specific embodiment, the second oligonucleotide is fluorescently labeled. In another specific embodiment, the plurality of second oligonucleotides are differentially fluorescently labeled. In a further specific embodiment, the detection step of said ligated molecule further comprises separation by size. In an additional specific embodiment, the size separation is by capillary electrophoresis.

In an additional object of the present invention, there is a method of analyzing at least one SNP from a plurality of individuals, comprising generating at least one specific nick translate molecule from DNA samples from each individual, wherein said specific nick translate molecule comprises the SNP; and detecting said SNP. In a specific embodiment, the detection step further comprises introducing to the nick translate molecule from the plurality of individuals a plurality of oligonucleotides, wherein said oligonucleotides hybridize adjacent to said SNP and wherein the 3′ base of said oligonucleotide is variable; extending by polymerization from said oligonucleotide, whereby extension only occurs if said variable 3′ base of said oligonucleotide is complementary to the corresponding nucleotide of said SNP; and detecting said extended oligonucleotide. In a specific embodiment, the method further comprises separating said extended oligonucleotides by size. In another specific embodiment, the size separation is by electrophoresis. In an additional specific embodiment, the extended oligonucleotides are detected by fluorescent label. In a further specific embodiment, the detection step further comprises introducing to the nick translate molecules from the plurality of individuals a plurality of first oligonucleotides, wherein a first oligonucleotide hybridizes such that its 5′ end is adjacent to the SNP; extending said first oligonucleotide by primer extension to form a plurality of nick translate molecule-first oligonucleotide extension product hybrids; introducing to said plurality of hybrids a plurality of second oligonucleotides, wherein a second oligonucleotide hybridizes adjacent to the SNP and comprises a variable nucleotide 3′ end; and ligating the 3′ end of said second oligonucleotide to the 5′ end of said first oligonucleotide extension product, whereby said ligation occurs only if said variable nucleotide is complementary to said SNP, to form a ligated molecule of said second oligonucleotide and said first oligonucleotide extension product; and detecting said ligated molecule. In a specific embodiment, the detection step further comprises separating said ligated molecules by size. In another specific embodiment, the size separation is by electrophoresis. In a further specific embodiment, the extended oligonucleotides are detected by fluorescent label.

In another object of the present invention, there is a method of analyzing at least one SNP from DNA samples from a plurality of individuals, comprising generating from each of said DNA samples a specific nick translate molecule comprising said SNP, wherein an adaptor on one end of said nick translate molecule comprises a unique nucleic acid sequence; introducing to said nick translate molecules a two-part oligonucleotide, comprising a first part comprising nucleic acid sequence complementary to the unique nucleic acid sequence of said adaptor; and a second part comprising nucleic acid sequence complementary to nucleic acid sequence immediately 5′ to the SNP; whereby said introduction results in the hybridization of said two parts of the oligonucleotide to the respective complementary sequences of said nick translate molecule and results in the formation of a loop in said nick translate molecule to bring said two parts in proximity of each other; introducing to said two-part oligonucleotide differentially fluorescently labeled dideoxynucleotide triphosphates and DNA polymerase; incorporating into the two-part oligonucleotide the fluorescently labeled dideoxynucleotide triphosphate which is complementary to said SNP; and detecting said SNP. In a specific embodiment, the SNP detection step further comprises hybridization of said fluorescently labeled dideoxynucleotide triphosphate-incorporated two-part oligonucleotide to a solid support, wherein the solid support comprises multiple positions, wherein each position comprises a unique adaptor sequence. In a specific embodiment, the solid support is a chip.

In another object of the present invention, there is a method of amplification of a genome comprising a SNP of interest, comprising obtaining the genome; generating a plurality of nick translate molecules from said genome, wherein at least one nick translate molecule comprises the SNP of interest; and amplifying the SNP-containing nick translate molecule. In a specific embodiment, the method further comprises detection of said SNP. In a specific embodiment, the SNP is detected by microarray analysis, sequencing, hybridization, or a combination thereof. In a further specific embodiment, the method step regarding generating of the nick translate molecules comprises attaching upstream adaptor molecules to ends of DNA molecules in the genome to provide a nick translation initiation site; subjecting the DNA molecules to nick translation comprising DNA polymerization and 5′-3′ exonuclease activity to produce the nick translate molecules; and attaching downstream adaptor molecules to the nick translate molecules to produce adaptor attached nick translate molecules. The following drawings form part of the present specification and are included to further demonstrate certain aspects of the present invention. The invention may be better understood by reference to one or more of these drawings in combination with the detailed description of specific embodiments presented herein.

### BRIEF DESCRIPTION OF THE DRAWINGS

FIG. 1 illustrates preparation of the primary PENTAmer library.

FIG. 2 shows types of PENTAmer libraries.

FIG. 3 demonstrates multiplexed amplification and detection of multiple SNPs in one DNA sample.

FIG. 4 depicts multiplexed amplification and detection of one SNP in multiple DNA samples.

FIG. 5 shows library-specific nick-translation adaptor ALS for multiplexing different PENTAmer libraries.

FIG. 6 illustrates multipexed peparation/amplification of DNA samples for SNPs detection using PENTAmer technology.

FIG. 7 shows preparation of DNA for multiple loci SNP analysis by whole-genome amplification of PENTAmer libraries.

FIGS. 8A and 8B demonstrate specific primary PENTAmer isolation by 5′end ligation-mediated capture.

FIG. 9 shows the structure of the hairpin oligonucleotide H.

FIGS. 10A and 10B depict multiplexed specific primary PENTAmer isolation by 5′end ligation-mediated capture.

FIGS. 11A and 11B show reducing PENTAmer library complexity by ligation-mediated capture.

FIG. 12 illustrates a library of 1024 biotinylated octamer oligonucleotides with 5-base specificity.

FIGS. 13A and 13B show specific primary PENTAmer isolation by primer extension-capture.

FIGS. 14A and 14B demonstrates multiplexed specific primary PENTAmer isolation by primer extension-capture.

FIG. 15 shows sequence-specific selection primers for PENTAmer isolation by primer extension-capture.

FIGS. 16A and 16B illustrates one-base selection by primer-extension/affinity capture procedure.

FIG. 17 demonstrates reducing PENTAmer library complexity by primer extension/PCR with primer-selector A.

FIG. 18 shows specific primary PENTAmer isolation by PCR.

FIG. 19 illustrates multiplexed specific primary PENTAmer isolation by PCR.

FIG. 20 demonstrates reducing PENTAmer library complexity by PCR with selective adaptor primers.

FIG. 21 depicts principles of circular recombinant PENTAmer construction and amplification of distal sequences using primers specific for proximal sequences.

FIG. 22 illustrates principles of making an ordered recombinant PENTAmer library.

FIG. 23 shows principles of making an unordered recombinant PENTAmer library.

FIG. 24A shows the use of nick-translation reactions to synthesize PENTAmers at both ends of DNA fragments for purposes of creating recombinant PENTAmers.

FIG. 24B demonstrates size fractionation and recombination steps to create an ordered recombinant PENTAmer library.

FIG. 24C depicts amplification of different tubes of an ordered recombinant PENTAmer library.

FIG. 25 illustrates the principle of amplifying an unordered recombinant PENTAmer library.

FIG. 26 shows the principle of making and amplifying an ordered recombinant PENTAmer library.

FIG. 27 demonstrates processing genomic DNA into an ordered PENTAmer library in a microwell plate and amplification of a large region of interest as ordered fragments.

FIG. 28 shows processing of genomic DNA into an unordered PENTAmer library in a single tube and amplification of a large region of interest as an unordered mixture of fragments.

FIG. 29 shows hybridization of locus-specific amplified PENTAmers to DNA microarray to detect SNPs in large region of interest.

FIG. 30 illustrates detection of multiple SNPs in one DNA sample using selective primer extension assay and size separation.

FIG. 31 demonstrates detection of multiple SNPs in one DNA sample using primer extension/selective ligation assay and size separation.

FIG. 32 shows multiplexed analysis of several SNPs in multiple DNA samples using size separation display.

FIGS. 33A and 33B illustrate detection of one SNP in multiple DNA samples one base primer extension-labeling reaction and hybridization to the oligo-chip.

### DETAILED DESCRIPTION OF THE INVENTION

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

As used herein the specification, “a” or “an” may mean one or more. As used herein in the claim(s), when used in conjunction with the word “comprising”, the words “a” or “an” may mean one or more than one. As used herein “another” may mean at least a second or more.

This application incorporates by reference herein in their entirety both U.S. patent application Ser. No. 09/860,738, filed May 18, 2001 and U.S. patent application Ser. No. 09/999,018, filed Nov. 15, 2001.

I. Generation of a Nick Translate Molecule

The present invention is directed to chromosome walking through the generation of nick translate molecules, and a skilled artisan recognizes that the nick translate molecules may be generated by any standard means in the art. However, in a preferred embodiment, the nick translate molecules are adaptor attached nick translate molecules (designated a PENTAmer).

The method for creating an adaptor attached nick translate molecule provides a powerful tool useful in overcoming many of the difficulties currently faced in large scale DNA manipulation, particularly genomic sequencing.

A. Primary PENTAmer

In the simplest implementation, a primary PENTAmer is generated by:

1) Ligating a nick-translation first adaptor to the proximal end of the source DNA (the template);

2) Initiating a nick translation reaction at the nick site of said adaptor using a DNA polymerase having 5′→3′ exonuclease activity;

3) Elongating the PENT product a specific time; and

4) Appending a nick-ligation second adaptor to the distal, 3′ end of the PENT product to form a PENTAmer-template hybrid (“nascent PENTAmer”).

While this basic technique sets forth the primary methodology envisioned by the inventors to create a PENTAmer product, it would be clear to one of ordinary skill that changes could be made in order to achieve an analogous outcome.

In a specific embodiment, the PENT reaction is initiated, continued, and terminated on a largely double-stranded template, which gives the PENTAmer amplification important advantages for creating DNA for sequence analysis. An advantage of using PENTAmers to amplify different regions of the template is the fact that in most applications PENTAmers having different internal sequences have the same terminal sequences. These advantages are important for creating PENTAmers that are most useful as intermediates for in vitro or in vivo amplification. Amplification of these intermediates is more useful than direct amplification of DNA by cloning or PCR.

During later steps, the PENTAmers can be degraded by incorporating distinguishable nucleotides during the reaction. For example, incorporation of dU nucleotides and subsequent exposure to dU-glycosylase allows destruction of the PENTAmers for separation from, for example, a desired nucleic molecule lacking the dU nucleotides.

The initiation site for a PENT reaction (as distinct from an oligonucleotide primer) can be introduced by any method that results in a free 3′ OH group on one side of a nick or gap in otherwise double-stranded DNA, including, but not limited to such groups introduced by: a) digestion by a restriction enzyme under conditions that only one strand of the double-stranded DNA template is hydrolyzed; b) random nicking by a chemical agent or an endonuclease such as DNAase I; c) nicking by f1 gene product II or homologous enzymes from other filamentous bacteriophage (Meyer and Geider, 1979); and/or d) chemical nicking of the template directed by triple-helix formation (Grant and Dervan, 1996).

However, for PENTAmer synthesis, the primary means of initiation is through the ligation of an oligonucleotide primer onto the target nucleic acid. This very powerful and general method to introduce an initiation site for strand replacement synthesis employs a panel of special double-stranded oligonucleotide adaptors designed specifically to be ligated to the termini produced by restriction enzymes. Each of these adaptors is designed such that the 3′ end of the restriction fragment to be sequenced can be covalently joined (ligated) to the adaptor, but the 5′ end cannot. Thus the 3′ end of the adaptor remains as a free 3′ OH at a 1 nucleotide gap in the DNA, which can serve as an initiation site for the strand-replacement sequencing of the restriction fragment. Because the number of different 3′ and 5′ overhanging sequences that can be produced by all restriction enzymes is finite, and the design of each adaptor will follow the same simple strategy, above, the design of every one of the possible adaptors can be foreseen, even for restriction enzymes that have not yet been identified. To facilitate sequencing, a set of such adaptors for strand replacement initiation can be synthesized with labels (radioactive, fluorescent, or chemical) and incorporated into the dideoxyribonucleotide-terminated strands to facilitate the detection of the bands on sequencing gels.

More specifically, adaptors with 5′ and 3′ extensions can be used in combination with restriction enzymes generating 2-base, 3-base and 4-base (or more) overhangs. The sense strand of the adaptor has a 5′ phosphate group that can be efficiently ligated to the restriction fragment to be sequenced. The anti-sense strand (bottom, underlined) is not phosphorylated at the 5′ end and is missing one base at the 3′ end, effectively preventing ligation between adaptors. This gap does not interfere with the covalent joining of the sense strand to the restriction fragment, and leaves a free 3′ OH site in the anti-sense strand for initiation of strand replacement synthesis.

Polymerization may be terminated specific distances from the priming site by inhibiting the polymerase a specific time after initiation. For example, under specific conditions Taq DNA polymerase is capable of strand replacement at the rate of 250 bases/min, so that arrest of the polymerase after 10 min occurs about 2500 bases from the initiation site. This strategy allows for pieces of DNA to be isolated from different locations in the genome.

PENT reactions may also be terminated by incorporation of a dideoxyribonucleotide instead of the homologous naturally-occurring nucleotide. This terminates growth of the new DNA strand at one of the positions that was formerly occupied by dA, dT, dG, or dC by incorporating ddA, ddT, ddG, or ddC. In principle, the reaction can be terminated using any suitable nucleotide analogs that prevent continuation of DNA synthesis at that site.

B. Secondary PENTAmers

Secondary PENTAmers are created by two nick-translation reactions. The length of the first PENT reaction determines the distance of one end of the secondary PENTAmer from the initiation position, whereas the second (shorter) PENT reaction determines the length of the secondary PENTAmer. The advantage of secondary PENTAmers is that the position of the PENTAmer within the template DNA and the length of the PENTAmer are independently controlled.

There are two methods to synthesize a secondary PENTAmer. In the first method, a secondary PENTAmer is created and amplified by:

Ligating a first terminus-attaching, nick translation adaptor to the proximal end of the template DNA molecule;

Initiating a first PENT reaction at the proximal end of the source DNA molecule using a first adaptor;

Elongating the first PENT product a specified time;

Appending a second nick-attaching adaptor to the distal, 3′ end of the first PENT product;

Initiating a second PENT reaction at the same proximal end of the source DNA molecule using the first adaptor;

Elongating the second PENT product a specifided time;

Appending a third nick-attaching adaptor to the 5′ end of the degraded first PENT product;

(Optionally) separating the single-stranded secondary PENTAmer of length from the template (e.g., by denaturation);

In a second method, a secondary PENTAmer is created by:

Ligating a first terminus-attaching, nick translation adaptor to the proximal end of the template DNA molecule;

Initiating a first PENT reaction at the proximal end of the source DNA molecule using the first adaptor;

Elongating the PENT product a specified time;

Appending a second nick-attaching adaptor to the distal, 3′ end of the PENT product;

Separating the single-stranded primary PENTAmer from the template;

Replicating the second strand of the primary PENTAmer using primer extension;

Initiating a second PENT reaction at the upstream end of the secondary PENTAmer;

Elongating the secondary PENT product a specified time;

Appending a third nick-attaching adaptor to the 3′ end of the secondary PENT product; and

(Optionally) separating the single-stranded secondary PENTAmer from the template.

C. Recombinant PENTAmers

The difficulty of immobilizing very large DNA fragments may be overcome by bringing together sequences from both the proximal and distal ends of long templates to create a recombinant PENTAmer.

A recombinant PENTAmer is made on a single template molecule, having different structures at the left (proximal) and right (distal) ends.

1) The first end of a recombination adaptor RA is attached to the left, proximal end of the template;

2) The second end of a recombination adaptor RA is attached to the right, distal end, to form a circular molecule; and

3) The initiation domain of adaptor RA is used to synthesize a PENTAmer containing the distal template sequences.

PENTAmers will only be created on those fragments that have been ligated to both ends of the recombination adaptor RA. Specific designs and use of recombination adaptors would be apparent to a skilled artisan. One embodiment uses an adaptor RA comprising a first ligation domain complementary to the proximal terminus of the template, an activatable second ligation domain complementary to the distal terminus, and a nick-translation initiation domain capable of translating the nick from the distal end toward the center of the template. In the case of a recombination adaptor of that specific design, the template would be made resistant to cleavage by the activation restriction enzyme by methylation at the restriction recognition sites, and the second step would be executed in the following way: 1) removal of unligated adaptor RA from solution, 2) activation of adaptor RA by restriction digestion of the unmethylated site within the adaptor, 3) dilution of the template, 4) ligation of the second ligation domain to the distal end of the template, and 5) concentration of the circularized molecules. Step 3 is executed by the same methods used to create a primary PENTAmer, however the nick-translation initiates at the initiation domain of an RA adaptor.

The PENTAmer formed can be amplified by any of the methods described earlier, e.g., by PCR using primers complementary to sequences in adaptors.

D. Adaptors

A preferred design of a nick-translation adaptor is formed by annealing 3 oligonucleotides (or more): oligonucleotide 1, oligonucleotide 2 and oligonucleotide 3. The left ends of these adaptors are designed to be ligated to double-stranded ends of template DNA molecules and used to initiate nick-translation reactions. Oligonucleotide 1 has a phosphate group (P) at the 5′ end and a blocking nucleotide at the 3′ end, a non-specified nucleotide composition and length from about 10 to 200 bases. Oligonucleotide 2 has a blocked 3′ end, a non-phosphorylated 5′ end, a nucleotide sequence complementary to the 5′ part of oligonucleotide 1 and length from about 5 to 195 bases. When hybridized together, oligonucleotides 1 and 2 form a double-stranded end designed to be ligated to the 3′ strand at the end of a template molecule. To be compatible with a ligation reaction to the end of a DNA restriction fragment, a nick-translation adaptor can have blunt, 5′-protruding or 3′-protruding end. Oligonucleotide 3 has a 3′ hydroxyl group, a non-phosphorylated 5′ end, a nucleotide sequence complementary to the 3′ part of oligonucleotide 1, and length from about 5 to 195 bases. When hybridized to oligonucleotide 1, oligonucleotides 2 and 3 form a nick or a few base gap within the lower strand of the adaptor. Oligonucleotide 3 can serve as a primer for initiation of the nick-translation reaction.

Other nick-attaching adaptors are partially double-stranded or completely single-stranded short DNA molecules that can be covalently linked to the 3′ hydroxyl group of the nick-translation DNA product. Nick-translation DNA product can be a single-stranded molecule isolated from its DNA template or the nick-translation product still hybridized to the template DNA. The nick-attaching adaptors are designed to complete the synthesis of the 3′ end of PENTAmers.

The next sections provide a brief overview of materials and techniques that a person of ordinary skill would deem important to the practice of the invention. These sections are followed by a more detailed description of the various embodiments of the invention.

II. Nucleic Acids

Genes are sequences of DNA in an organism's genome encoding information that is converted into various products making up a whole cell. They are expressed by the process of transcription, which involves copying the sequence of DNA into RNA. Most genes encode information to make proteins, but some encode RNAs involved in other processes. If a gene encodes a protein, its transcription product is called mRNA (“messenger” RNA). After transcription in the nucleus (where DNA is located), the mRNA must be transported into the cytoplasm for the process of translation, which converts the code of the mRNA into a sequence of amino acids to form protein. In order to direct transport into the cytoplasm, the 3′ ends of mRNA molecules are post-transcriptionally modified by addition of several adenylate residues to form the “polyA” tail. This characteristic modification distinguishes gene expression products destined to make protein from other molecules in the cell, and thereby provides one means for detecting and monitoring the gene expression activities of a cell.

The term “nucleic acid” will generally refer to at least one molecule or strand of DNA, RNA or a derivative or mimic thereof, comprising at least one nucleobase, such as, for example, a naturally occurring purine or pyrimidine base found in DNA (e.g. adenine “A,” guanine “G,” thymine “T” and cytosine “C”) or RNA (e.g. A, G, uracil “U” and C). The term “nucleic acid” encompass the terms “oligonucleotide” and “polynucleotide.” The term “oligonucleotide” refers to at least one molecule of between about 3 and about 100 nucleobases in length. The term “polynucleotide” refers to at least one molecule of greater than about 100 nucleobases in length. These definitions generally refer to at least one single-stranded molecule, but in specific embodiments will also encompass at least one additional strand that is partially, substantially or fully complementary to the at least one single-stranded molecule. Thus, a nucleic acid may encompass at least one double-stranded molecule or at least one triple-stranded molecule that comprises one or more complementary strand(s) or “complement(s)” of a particular sequence comprising a strand of the molecule. As used herein, a single stranded nucleic acid may be denoted by the prefix “ss”, a double stranded nucleic acid by the prefix “ds”, and a triple stranded nucleic acid by the prefix “ts.”

Nucleic acid(s) that are “complementary” or “complement(s)” are those that are capable of base-pairing according to the standard Watson-Crick, Hoogsteen or reverse Hoogsteen binding complementarity rules. As used herein, the term “complementary” or “complement(s)” also refers to nucleic acid(s) that are substantially complementary, as may be assessed by the same nucleotide comparison set forth above. The term “substantially complementary” refers to a nucleic acid comprising at least one sequence of consecutive nucleobases, or semiconsecutive nucleobases if one or more nucleobase moieties are not present in the molecule, are capable of hybridizing to at least one nucleic acid strand or duplex even if less than all nucleobases do not base pair with a counterpart nucleobase. In certain embodiments, a “substantially complementary” nucleic acid contains at least one sequence in which about 70%, about 71%, about 72%, about 73%, about 74%, about 75%, about 76%, about 77%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, to about 100%, and any range therein, of the nucleobase sequence is capable of base-pairing with at least one single or double stranded nucleic acid molecule during hybridization. In certain embodiments, the term “substantially complementary” refers to at least one nucleic acid that may hybridize to at least one nucleic acid strand or duplex in stringent conditions. In certain embodiments, a “partly complementary” nucleic acid comprises at least one sequence that may hybridize in low stringency conditions to at least one single or double stranded nucleic acid, or contains at least one sequence in which less than about 70% of the nucleobase sequence is capable of base-pairing with at least one single or double stranded nucleic acid molecule during hybridization.

As used herein, “hybridization”, “hybridizes” or “capable of hybridizing” is understood to mean the forming of a double or triple stranded molecule or a molecule with partial double or triple stranded nature. The term “hybridization”, “hybridize(s)” or “capable of hybridizing” encompasses the terms “stringent condition(s)” or “high stringency” and the terms “low stringency” or “low stringency condition(s).”

As used herein “stringent condition(s)” or “high stringency” are those that allow hybridization between or within one or more nucleic acid strand(s) containing complementary sequence(s), but precludes hybridization of random sequences. Stringent conditions tolerate little, if any, mismatch between a nucleic acid and a target strand. Such conditions are well known to those of ordinary skill in the art, and are preferred for applications requiring high selectivity. Non-limiting applications include isolating at least one nucleic acid, such as a gene or nucleic acid segment thereof, or detecting at least one specific mRNA transcript or nucleic acid segment thereof, and the like.

Stringent conditions may comprise low salt and/or high temperature conditions, such as provided by about 0.02 M to about 0.15 M NaCl at temperatures of about 50° C. to about 70° C. It is understood that the temperature and ionic strength of a desired stringency are determined in part by the length of the particular nucleic acid(s), the length and nucleobase content of the target sequence(s), the charge composition of the nucleic acid(s), and to the presence of formamide, tetramethylammonium chloride or other solvent(s) in the hybridization mixture. It is generally appreciated that conditions may be rendered more stringent, such as, for example, the addition of increasing amounts of formamide.

It is also understood that these ranges, compositions and conditions for hybridization are mentioned by way of non-limiting example only, and that the desired stringency for a particular hybridization reaction is often determined empirically by comparison to one or more positive or negative controls. Depending on the application envisioned it is preferred to employ varying conditions of hybridization to achieve varying degrees of selectivity of the nucleic acid(s) towards target sequence(s). In a non-limiting example, identification or isolation of related target nucleic acid(s) that do not hybridize to a nucleic acid under stringent conditions may be achieved by hybridization at low temperature and/or high ionic strength. Such conditions are termed “low stringency” or “low stringency conditions”, and non-limiting examples of low stringency include hybridization performed at about 0.15 M to about 0.9 M NaCl at a temperature range of about 20° C. to about 50° C. Of course, it is within the skill of one in the art to further modify the low or high stringency conditions to suite a particular application.

As used herein a “nucleobase” refers to a naturally occurring heterocyclic base, such as A, T, G, C or U (“naturally occurring nucleobase(s)”), found in at least one naturally occurring nucleic acid (i.e. DNA and RNA), and their naturally or non-naturally occurring derivatives and mimics. Non-limiting examples of nucleobases include purines and pyrimidines, as well as derivatives and mimics thereof, which generally can form one or more hydrogen bonds (“anneal” or “hybridize”) with at least one naturally occurring nucleobase in manner that may substitute for naturally occurring nucleobase pairing (e.g. the hydrogen bonding between A and T, G and C, and A and U).

As used herein, a “nucleotide” refers to a nucleoside further comprising a “backbone moiety” generally used for the covalent attachment of one or more nucleotides to another molecule or to each other to form one or more nucleic acids. The “backbone moiety” in naturally occurring nucleotides typically comprises a phosphorus moiety, which is covalently attached to a 5-carbon sugar. The attachment of the backbone moiety typically occurs at either the 3′- or 5′-position of the 5-carbon sugar. However, other types of attachments are known in the art, particularly when the nucleotide comprises derivatives or mimics of a naturally occurring 5-carbon sugar or phosphorus moiety, and non-limiting examples are described herein.

III. Restriction Enzymes

Restriction-enzymes recognize specific short DNA sequences four to eight nucleotides long (see Table I), and cleave the DNA at a site within this sequence. In the context of the present invention, restriction enzymes are used to cleave DNA molecules at sites corresponding to various restriction-enzyme recognition sites. The site may be specifically modified to allow for the initiation of the PENT reaction. In another embodiment, if the sequence of the recognition site is known primers can be designed comprising nucleotides corresponding to the recognition sequences. These primers, further comprising PENT initiation sites may be ligated to the digested DNA.

Restriction-enzymes recognize specific short DNA sequences four to eight nucleotides long (see Table I), and cleave the DNA at a site within this sequence. In the context of the present invention, restriction enzymes are used to cleave cDNA molecules at sites corresponding to various restriction-enzyme recognition sites. Frequently cutting enzymes, such as the four-base cutter enzymes, are preferred as this yields DNA fragments that are in the right size range for subsequent amplification reactions. Some of the preferred four-base cutters are N1aIII, DpnII, Sau3AI, Hsp921I, MboI, NdeII, Bsp1431, Tsp509 I, HhaI, HinP1I, HpaII, MspI, Taq alphaI, MaeII or K2091.

As the sequence of the recognition site is known (see list below), primers can be designed comprising nucleotides corresponding to the recognition sequences. If the primer sets have in addition to the restriction recognition sequence, degenerate sequences corresponding to different combinations of nucleotide sequences, one can use the primer set to amplify DNA fragments that have been cleaved by the particular restriction enzyme. The list below exemplifies the currently known restriction enzymes that may be used in the invention.

TABLE I
RESTRICTION ENZYMES

| | |
|---|---|
| Enzyme Name | Recognition Sequence |

| | | |
|---|---|---|
| Aat II | GACGTC | |
| Acc65 I | GGTACC | |
| Acc I | GTMKAC | |
| Aci I | CCGC | |
| Acl I | AACGTT | |
| Afe I | AGCGCT | |
| Afl II | CTTAAG | |
| Afl III | ACRYGT | |
| Age I | ACCGGT | |
| Ahd I | GACNNNNNGTC | (SEQ ID NO:1) |
| Alu 1 | AGCT | |
| Alw I | GGATC | |
| AlwN I | CAGNNNCTG | (SEQ ID NO:2) |
| Apa I | GGGCCC | |
| ApaL I | GTGCAC | |
| Apo I | RAATTY | |
| Asc I | GGCGCGCC | |
| Ase I | ATTAAT | |
| Ava I | CYCGRG | |
| Ava II | GGWCC | |
| Avr II | CCTAGG | |
| Bae I | NACNNNNGTAPyCN | (SEQ ID NO:3) |
| BamH I | GGATCC | |
| Ban I | GGYRCC | |
| Ban II | GRGCYC | |
| Bbs I | GAAGAC | |
| Bbv I | GCAGC | |
| BbvC I | CCTCAGC | |
| Bcg I | CGANNNNNNTGC | (SEQ ID NO:4) |
| BciV I | GTATCC | |
| Bcl I | TGATCA | |
| Bfa I | CTAG | |
| Bgl I | GCCNNNNTGC | (SEQ ID NO:5) |
| Bgl II | AGATCT | |
| Blp I | GCTNAGC | |
| Bmr I | ACTGGG | |
| Bpm I | CTGGAG | |
| BsaA I | YACGTR | |
| BsaB I | GATNNNNATC | (SEQ ID NO:6) |
| BsaH I | GRCGYC | |
| Bsa I | GGTCTC | |
| BsaJ I | CCNNGG | |
| BsaW I | WCCGGW | |
| BseR I | GAGGAG | |
| Bsg I | GTGCAG | |
| BsiE I | CGRYCG | |
| BsiHKA I | GWGCWC | |
| BsiW I | CGTACG | |
| Bsl I | CCNNNNNNNGG | (SEQ ID NO:7) |
| BsmA I | GTCTC | |
| BsmB I | CGTCTC | |
| BsmF I | GGGAC | |
| Bsm I | GAATGC | |
| BsoB I | CYCGRG | |
| Bsp1286 I | GDGCHC | |
| BspD I | ATCGAT | |
| BspE I | TCCGGA | |
| BspH I | TCATGA | |
| BspM I | ACCTGC | |
| BsrB I | CCGCTC | |
| BsrD I | GCAATG | |
| BsrF I | RCCGGY | |
| BsrG I | TGTACA | |
| Bsr I | ACTGG | |
| BssH II | GCGCGC | |
| BssK I | CCNGG | |
| Bst4C I | ACNGT | |
| BssS I | CACGAG | |
| BstAP I | GCANNNNNTGC | (SEQ ID NO:8) |
| BstB I | TTCGAA | |
| BstE II | GGTNACC | |
| BstF5 I | GGATGNN | |
| BstN I | CCWGG | |
| BstU I | CGCG | |
| BstX I | CCANNNNNNTGG | (SEQ ID NO:9) |
| BstY I | RGATCY | |
| BstZ17 I | GTATAC | |
| Bsu36 I | CCTNAGG | |
| Btg I | CCPuPyGG | |
| Btr I | CACGTG | |
| Cac8 I | GCNNGC | |
| Cla I | ATCGAT | |
| Dde I | CTNAG | |
| Dpn I | GATC | |
| Dpn II | GATC | |
| Dra I | TTTAAA | |
| Dra III | CACNNNGTG | (SEQ ID NO:10) |
| Drd I | GACNNNNNNGTC | (SEQ ID NO:11) |
| Eae I | YGGCCR | |
| Eag I | CGGCCG | |
| Ear I | CTCTTC | |
| Eci I | GGCGGA | |
| EcoN I | CCTNNNNNAGG | (SEQ ID NO:12) |
| EcoO109 I | RGGNCCY | |
| EcoR I | GAATTC | |
| EcoR V | GATATC | |
| Fau I | CCCGCNNNN | |
| Fnu4H I | GCNGC | |
| Fok I | GGATG | |
| Fse I | GGCCGGCC | |
| Fsp I | TGCGCA | |
| Hae II | RGCGCY | |
| Hae III | GGCC | |
| Hga I | GACGC | |
| Hha I | GCGC | |
| Hinc II | GTYRAC | |
| Hind III | AAGCTT | |
| Hinf I | GANTC | |
| HinPl I | GCGC | |
| Hpa I | GTTAAC | |
| Hpa II | CCGG | |
| Hph I | GGTGA | |
| Kas I | GGCGCC | |
| Kpn I | GGTACC | |
| Mbo I | GATC | |
| Mbo II | GAAGA | |
| Mfe I | CAATTG | |
| Mlu I | ACGCGT | |
| Mly I | GAGTCNNNNN | (SEQ ID NO:13) |
| Mnl I | CCTC | |
| Msc I | TGGCCA | |
| Mse I | TTAA | |
| Msl I | CAYNNNNRTG | (SEQ ID NO:14) |
| MspAl I | CMGCKG | |
| Msp I | CCGG | |
| Mwo I | GCNNNNNNNGC | (SEQ ID NO:15) |
| Nae I | GCCGGC | |
| Nar I | GGCGCC | |
| Nci I | CCSGG | |
| Nco I | CCATGG | |
| Nde I | CATATG | |
| NgoMI V | GCCGGC | |
| Nhe I | GCTAGC | |
| Nla III | CATG | |
| Nla IV | GGNNCC | |
| Not I | GCGGCCGC | |
| Nru I | TCGCGA | |
| Nsi I | ATGCAT | |
| Nsp I | RCATGY | |
| Pac I | TTAATTAA | |
| PaeR7 I | CTCGAG | |
| Pci I | ACATGT | |
| PflF I | GACNNNGTC | |
| PflM I | CCANNNNNTGG | (SEQ ID NO:16) |
| Ple I | GAGTC | |
| Pme I | GTTTAAAC | |
| Pml I | CACGTG | |
| PpuM I | RGGWCCY | |
| PshA I | GACNNNNGTC | (SEQ ID NO:17) |
| Psi I | TTATAA | |
| PspG I | CCWGG | |
| PspOM I | GGGCCC | |
| Pst I | CTGCAG | |
| Pvu I | CGATCG | |
| Pvu II | CAGCTG | |
| Rsa I | GTAC | |
| Rsr II | CGGWCCG | |
| Sac I | GAGCTC | |
| Sac II | CCGCGG | |
| Sal I | GTCGAC | |
| Sap I | GCTCTTC | |
| Sau3A I | GATC | |
| Sau96 I | GGNCC | |
| Sbf I | CCTGCAGG | |
| Sca I | AGTACT | |
| ScrF I | CCNGG | |
| SexA I | ACCWGGT | |
| SfaN I | GCATC | |
| Sfc I | CTRYAG | |
| Sfi I | GGCCNNNNNGGCC | (SEQ ID NO:18) |
| Sfo I | GGCGCC | |
| SgrA I | CRCCGGYG | |
| Sma I | CCCGGG | |
| Smi I | CTYRAG | |
| SnaB I | TACGTA | |
| Spe I | ACTAGT | |
| Sph I | GCATGC | |
| Ssp I | AATATT | |
| Stu I | AGGCCT | |
| Sty I | CCWWGG | |
| Swa I | ATTTAAAT | |
| Taq I | TCGA | |
| Tfi I | GAWTC | |
| Tli I | CTCGAG | |
| Tse I | GCWGC | |
| Tsp45 I | GTSAC | |
| Tsp509 I | AATT | |
| TspR I | CAGTG | |
| Tth111 I | GACNNNGTC | |
| Xba I | TCTAGA | |
| Xcm I | CCANNNNNNNNNTGG | (SEQ ID NO:19) |
| Xho I | CTCGAG | |
| Xma I | CCCGGG | |
| Xmn I | GAANNNNTTC | (SEQ ID NO:20) |

Furthermore, a skilled artisan recognizes that it may be useful in the present invention to selectively render particular restriction enzyme sites uncleavable, such as by methylation of the recognition site prior to exposure to certain methylation-sensitive restriction enzymes. A skilled artisan recognizes that, for example, the dam and dcm genes of E. coli encode gene products which are methylases that methylate a nucleic acid in their specific recognition sequence. Some enzymes will not cleave methylated sites, whereas other enzymes, such as Dpn I, have a requirement for methylation at the recognition site. Examples of different classes of methylation requirements for specific enzymes are in Table II as follows:

TABLE II
CpG METHYLATION AND ENZYME CLEAVAGE
Cleavage Blocked at All Sites

| | | | | | | | |
|---|---|---|---|---|---|---|---|
| Aat II | GACGTC | BsrF I | RCCGGY | Hae II | RGCGCY | Nru I | TCGCGA |
| Aci I | CCGC | BSSH II | GCGCGC | Hga I | GACGC | Pml I | CACGTG |
| Age I | ACCGGT | BSTB I | TTCGAA | Hha I | GCGC | Psp1406 I | AACGTT |
| Aha II | GRGGYC | BSTU I | CGCG | HinPl I | GCGC | Pvu I | CGATCG |
| Asc I | GGCGCGCC | Cfr10 I | RCCGGY | Hpa II | CCGG | Rsr II | CGGWCCG |
| Ava I | CYCGRG | Cla I | ATCGAT | Kas I | GGCGCC | Sac II | CCGCGG |
| BsaA I | YAGGTR | Eag I | CGGCCG | Mlu I | ACGCGT | Sal I | GTCGAG |
| BsaH I | GRCGYC | Eco47 III | AGCGCT | Nae I | GCCGGC | Sma I | CCCGGG |
| BsiE I | CGRYCG | Esp3 I | CGTCTC(1/5) | Nar I | GGCGCC | SnaB I | TACGTA |
| BsiW I | CGTACG | Fse I | GGCCGGCC | NgoM IV | GCCGGC | Tai I | ACGT |
| BspD I | ATCGAT | Fsp I | TGCGCA | Not I | GCGGCCGC | Xho I | CTCGAG |

Cleavage Blocked Only at Sites with Overlapping CG

| | | | | | | | |
|---|---|---|---|---|---|---|---|
| Acc I | GTMKAC | Ban I³ | GGYRCC | Bsp120 I | GGGCCC | Nhe I | GCTAGC |
| Acc65 I | GGTACC | BsaB I² | GATN4ATC | Bstl107 I | GTATAC | Rsa I³ | GTAC |
| | | | (SEQ ID NO:21) | | | | |
| Alw26 I | GTCTC | Bsg I | GTGCAG | Drd I¹ | GACN6GTC | PshA I³ | GACNNNNGTC |
| | | | | | (SEQ ID NO:23) | | (SEQ ID NO:24) |
| Apa I | GGGCCC | Bsl I | CCN7GG | Eae I | YGGCCR | Sau3A I | GATC |
| | | | (SEQ ID NO:22) | | | | |
| ApaL I | GTGCAC | BsmA I | GTCTG | Ecl136 II | GAGCTC | Sau96 I | GGNCC |
| Ava II | GGWCC | BsoF I¹ | GCNGC | Hpa I³ | GTTAAC | | |

Cleavage Not Blocked at Sites with Overlapping CG

| | | | | | | | |
|---|---|---|---|---|---|---|---|
| BamH I | GGATCC | BsrB I² | GAGCGG | EcoR V | GATATC | Pme I | GTTAAAC |
| Ban II | GRGCYC | BstE II | GGTNACC | Fok I | GGATG | Sad | GAGCTC |
| Bbs I | GAAGAC | BstY I | RGTACY | Hae III | GGCC | StaN I | GCATC |
| BsaJ I | CCNNGG | Csp6 I | GTAC | HglA I | GWGCWC | Sph I | GCATGC |
| BsaW I | WCCGGW | Eam11051 | GACN5GTC | Hph I | GGTGA | Taq I | TCGA |
| | | | (SEQ ID NO:25) | | | | |
| Bsm I | GATTGC | Ear I | CCTCTTC | Kpn I | GGTACC | Tfi I | GAWTC |
| Bsp1286 I | GDGCHC | EcoO1091 | RGGNCCY | Msp I | CCGG | Tth111 I | GACN3GTC |
| BspE I² | TCCGGA | EcoR I | GATTC | PaeR7 I | CTCGAG | Xma I | CCCGGG |
| BspM I | ACCTGC | | | | | | |

Examples of restriction enzyme sites sensitive to Dam and Dcm methylation in particular are in Table III as follows:

TABLE III
DAM AND DCM METHYLATION

Dam Methylation: G^{m}ATC
Blocked by Overlapping Dam:

| | |
|---|---|
| Alw I | GGATC |
| Bcl I | TGATCA |
| BsaB I | GATCNNNATC |
| BspD I | ATCGATC |
| BspH I | TCCGGATC |
| BspH I | TCATGATC |
| Cla I | ATCGATC |
| Dpn II | GATC |
| Hph I | GGTGATC |
| Mbo I | GATC |
| Mbo II | GAAGATC |
| Nru I | TCGCGATC |
| Taq I | TCGATC |
| Xba I | TCTAGATC |

Not Blocked by Overlapping Dam:

| | |
|---|---|
| BamH I | GGATCC |
| Bgl II | AGATCT |
| BspM II | TCCGGATC |
| BstY I | (A/G)GATC(C/T) |
| Pvu I | CGATCG |
| Sau3A I | GATC |

Dcm Methylation: C^{m}C(A/T)GG
Blocked by Overlapping Dcm:

| | |
|---|---|
| Acc65 I | GGTACC(A/T)GG |
| AlwN I | CAGNNCCTGG |
| Apa I | GGGCCC(A/T)GG |
| Ava II | GG(A/T)CC(A/T)GG |
| Bal I | TGGCCAGg |
| Bpm I | CCTGGAG |
| Bsl I | CC(A/T)GGNNNNGG |
| Bsp120 I | GGGCCC(A/T)GG |
| BssK I | CC(A/T)GG |
| Eae I | (C/T)GGCCAGG |
| EcoO109 I | (A/G)GGNCCTGG |
| EcoR II | CC(A/T)GG |
| Msc I | TGGCCAGG |
| PflM I | CCAGGNNNTGG |
| PpuM I | (A/G)GG(A/T)CCTGG |
| Sau96 I | GGNCC(A/T)GG |
| ScrF I | CC(A/T)GG |
| SexA I | ACC(A/T)GGT |
| Sfi I | GGCC(A/T)GGNNGGCC |
| Stu I | AGGCCTGG |

Not Blocked by Overlapping Dcm

| | |
|---|---|
| Ban II | G(A/G)GCCC(A/T)GG |
| Bgl I | GCC(A/T)GGNNGGC |
| BsaJ I | CC(A/T)GGG |
| Bsp1286 I | G(A/G/T)GCCC(A/T)GG |
| BstN I | CC(A/T)GG |
| BstE II | GGTNACC(A/T)GG |
| Ehe I | GGCGCC(A/T)GG |
| Hae III | GGCC(A/T)GG |
| Kpn I | GGTACC(A/T)(GG |
| Nar I | GGCGCC(A/T)GG |
| Sfi I | GGCCNNNNNGGCC(A/T)GG |

Other examples of methylation-sensitive enzymes, which may not be listed here, are obtainable by a skilled artisan.

IV. Other Enzymes

Other enzymes that may be used in conjunction with the invention include nucleic acid modifying enzymes listed in the following tables.

TABLE IV
POLYMERASES AND REVERSE TRANSCRIPTASES

Thermostable DNA Polymerases:
OmniBase ™ Sequencing Enzyme
Pfu DNA Polymerase
Taq DNA Polymerase
Taq DNA Polymerase, Sequencing Grade
TaqBead ™ Hot Start Polymerase
AmpliTaq Gold
Tfl DNA Polymerase
Tli DNA Polymerase
Tth DNA Polymerase
DNA Polymerases:
DNA Polymerase I, Klenow Fragment, Exonuclease Minus
DNA Polymerase I
DNA Polymerase I Large (Klenow) Fragment
Terminal Deoxynucleotidyl Transferase
T4 DNA Polymerase
Reverse Transcriptases:
AMV Reverse Transcriptase
M-MLV Reverse Transcriptase

TABLE V
DNA/RNA MODIFYING ENZYMES

Ligases:
T4 DNA Ligase
Kinases
T4 Polynucleotide Kinase

V. DNA Polymerases

In the context of the present invention it is generally contemplated that the DNA polymerase will retain 5′-3′ exonuclease activity. Nevertheless, it is envisioned that the methods of the invention could be carried out with one or more enzymes where multiple enzymes combine to carry out the function of a single DNA polymerase molecule retaining 5′-3′ exonuclease activity. Effective polymerases which retain 5′-3′ exonuclease activity include, for example, E. coli DNA polymerase I, Taq DNA polymerase, S. pneumoniae DNA polymerase I, Tfl DNA polymerase, D. radiodurans DNA polymerase I, Tth DNA polymerase, Tth XL DNA polymerase, M. tuberculosis DNA polymerase I, M. thermoautotrophicum DNA polymerase I, Herpes simplex-1 DNA polymerase, E. coli DNA polymerase I Klenow fragment, Vent DNA polymerase, thermosequenase and wild-type or modified T7 DNA polymerases. In preferred embodiments, the effective polymerase is E. coli DNA polymerase I, M. tuberculosis DNA polymerase I or Taq DNA polymerase.

Where the break in the substantially double stranded nucleic acid template is a gap of at least a base or nucleotide in length that comprises, or is reacted to comprise, a 3′ hydroxyl group, the range of effective polymerases that may be used is even broader. In such aspects, the effective polymerase may be, for example, E. coli DNA polymerase I, Taq DNA polymerase, S. pneumoniae DNA polymerase I, Tfl DNA polymerase, D. radiodurans DNA polymerase I, Tth DNA polymerase, Tth XL DNA polymerase, M. tuberculosis DNA polymerase I, M. thermoautotrophicum DNA polymerase I, Herpes simplex-1 DNA polymerase, E. coli DNA polymerase I Klenow fragment, T4 DNA polymerase, vent DNA polymerase, thermosequenase or a wild-type or modified T7 DNA polymerase. In preferred aspects, the effective polymerase is E. coli DNA polymerase I, M tuberculosis DNA polymerase I, Taq DNA polymerase or T4 DNA polymerase.

VI. Hybridization

PENTAmer synthesis requires the use of primers which hybridize to specific sequences. Further, PENT reaction products may be useful as probes in hybridization analysis. The use of a probe or primer of between about 13 and 100 nucleotides, preferably between about 17 and 100 nucleotides in length, or in some aspects of the invention up to about 1-2 Kb or more in length, allows the formation of a duplex molecule that is both stable and selective. Molecules having complementary sequences over contiguous stretches greater than about 20 bases in length are generally preferred, to increase stability and/or selectivity of the hybrid molecules obtained. One will generally prefer to design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

Depending on the application envisioned, one would desire to employ varying conditions of hybridization to achieve varying degrees of selectivity of the probe or primers for the target sequence. For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

Conditions may be rendered less stringent by increasing salt concentration and/or decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Hybridization conditions can be readily manipulated depending on the desired results.

In other embodiments, hybridization may be achieved under conditions of, for example, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

VII. Amplification Of Nucleic Acids

Nucleic acids useful as templates for amplification may be isolated from cells, tissues or other samples according to standard methodologies (Sambrook et al., 1989). In certain embodiments, analysis is performed on whole cell or tissue homogenates or biological fluid samples without substantial purification of the template nucleic acid. The nucleic acid may be genomic DNA or fractionated or whole cell RNA. Where RNA is used, it may be desired to first convert the RNA to a complementary DNA.

The term “primer,” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred.

Pairs of primers designed to selectively hybridize to nucleic acids are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids contain one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

The amplification product may be detected or quantified. In certain applications, the detection may be performed by visual means. Alternatively, the detection may involve indirect identification of the product via chemiluminescence, radioactive scintigraphy of incorporated radiolabel or fluorescent label or even via a system using electrical and/or thermal impulse signals (Affymax technology).

A number of template dependent processes are available to amplify the oligonucleotide sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al., 1990, each of which is incorporated herein by reference in their entirety. Briefly, two synthetic oligonucleotide primers, which are complementary to two regions of the template DNA (one for each strand) to be amplified, are added to the template DNA (that need not be pure), in the presence of excess deoxynucleotides (dNTPs) and a thermostable polymerase, such as, for example, Taq (Thermus aquaticus) DNA polymerase. In a series (typically 30-35) of temperature cycles, the target DNA is repeatedly denatured (around 90° C.), annealed to the primers (typically at 50-60° C.) and a daughter strand extended from the primers (72° C.). As the daughter strands are created they act as templates in subsequent cycles. Thus the template region between the two primers is amplified exponentially, rather than linearly.

A reverse transcriptase PCR™ amplification procedure may be performed to quantify the amount of mRNA amplified. Methods of reverse transcribing RNA into cDNA are well known and described in Sambrook et al., 1989. Alternative methods for reverse transcription utilize thermostable DNA polymerases. These methods are described in WO 90/07641. Polymerase chain reaction methodologies are well known in the art. Representative methods of RT-PCR are described in U.S. Pat. No. 5,882,864.

A. LCR

Another method for amplification is the ligase chain reaction (“LCR”), disclosed in European Patent Application No. 320,308, incorporated herein by reference. In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR™, bound ligated units dissociate from the target and then serve as “target sequences” for ligation of excess probe pairs. U.S. Pat. No. 4,883,750, incorporated herein by reference, describes a method similar to LCR for binding probe pairs to a target sequence.

B. Qbeta Replicase

Qbeta Replicase, described in PCT Patent Application No. PCT/US87/00880, also may be used as still another amplification method in the present invention. In this method, a replicative sequence of RNA which has a region complementary to that of a target is added to a sample in the presence of an RNA polymerase. The polymerase will copy the replicative sequence which can then be detected.

C. Isothermal Amplification

An isothermal amplification method, in which restriction endonucleases and ligases are used to achieve the amplification of target molecules that contain nucleotide 5′-[α-thio]-triphosphates in one strand of a restriction site also may be useful in the amplification of nucleic acids in the present invention. Such an amplification method is described by Walker et al. 1992, incorporated herein by reference.

D. Strand Displacement Amplification

Strand Displacement Amplification (SDA) is another method of carrying out isothermal amplification of nucleic acids which involves multiple rounds of strand displacement and synthesis. A similar method, called Repair Chain Reaction (RCR), involves annealing several probes throughout a region targeted for amplification, followed by a repair reaction in which only two of the four bases are present. The other two bases can be added as biotinylated derivatives for easy detection. A similar approach is used in SDA.

E. Cyclic Probe Reaction

Target specific sequences can also be detected using a cyclic probe reaction (CPR). In CPR, a probe having 3′ and 5′ sequences of non-specific DNA and a middle sequence of specific RNA is hybridized to DNA which is present in a sample. Upon hybridization, the reaction is treated with RNase H, and the products of the probe identified as distinctive products which are released after digestion. The original template is annealed to another cycling probe and the reaction is repeated.

F. Transcription-Based Amplification

Other nucleic acid amplification procedures include transcription-based amplification systems (TAS), including nucleic acid sequence based amplification (NASBA) and 3SR, Kwoh et al, 1989; PCT Patent Application WO 88/10315 et al., 1989, each incorporated herein by reference).

In NASBA, the nucleic acids can be prepared for amplification by standard phenol/chloroform extraction, heat denaturation of a clinical sample, treatment with lysis buffer and minispin columns for isolation of DNA and RNA or guanidinium chloride extraction of RNA. These amplification techniques involve annealing a primer which has target specific sequences. Following polymerization, DNA/RNA hybrids are digested with RNase H while double stranded DNA molecules are heat denatured again. In either case the single stranded DNA is made fully double stranded by addition of second target specific primer, followed by polymerization. The double-stranded DNA molecules are then multiply transcribed by a polymerase such as T7 or SP6. In an isothermal cyclic reaction, the RNA's are reverse transcribed into double stranded DNA, and transcribed once against with a polymerase such as T7 or SP6. The resulting products, whether truncated or complete, indicate target specific sequences.

7. Other Amplification Methods

Other amplification methods, as described in British Patent Application No. GB 2,202,328, and in PCT Patent Application No. PCT/US89/01025, each incorporated herein by reference, may be used in accordance with the present invention. In the former application, “modified” primers are used in a PCR™ like, template and enzyme dependent synthesis. The primers may be modified by labeling with a capture moiety (e.g., biotin) and/or a detector moiety (e.g., enzyme). In the latter application, an excess of labeled probes are added to a sample. In the presence of the target sequence, the probe binds and is cleaved catalytically. After cleavage, the target sequence is released intact to be bound by excess probe. Cleavage of the labeled probe signals the presence of the target sequence.

Miller et al., PCT Patent Application WO 89/06700 (incorporated herein by reference) disclose a nucleic acid sequence amplification scheme based on the hybridization of a promoter/primer sequence to a target single-stranded DNA (“ssDNA”) followed by transcription of many RNA copies of the sequence. This scheme is not cyclic, i.e., new templates are not produced from the resultant RNA transcripts.

Other suitable amplification methods include “race” and “one-sided PCR™” (Frohman, 1990; Ohara et al., 1989, each herein incorporated by reference). Methods based on ligation of two (or more) oligonucleotides in the presence of nucleic acid having the sequence of the resulting “di-oligonucleotide”, thereby amplifying the di-oligonucleotide, also may be used in the amplification step of the present invention, Wu et al., 1989, incorporated herein by reference).

VIII. Detection of Nucleic Acids

Following any amplification, it may be desirable to separate the amplification product from the template and/or the excess primer. In one embodiment, amplification products are separated by agarose, agarose-acrylamide or polyacrylamide gel electrophoresis using standard methods (Sambrook et al., 1989). Separated amplification products may be cut out and eluted from the gel for further manipulation. Using low melting point agarose gels, the separated band may be removed by heating the gel, followed by extraction of the nucleic acid.

Separation of nucleic acids may also be effected by chromatographic techniques known in art. There are many kinds of chromatography which may be used in the practice of the present invention, including adsorption, partition, ion-exchange, hydroxylapatite, molecular sieve, reverse-phase, column, paper, thin-layer, and gas chromatography as well as HPLC.

In certain embodiments, the amplification products are visualized. A typical visualization method involves staining of a gel with ethidium bromide and visualization of bands under UV light. Alternatively, if the amplification products are integrally labeled with radio- or fluorometrically-labeled nucleotides, the separated amplification products can be exposed to x-ray film or visualized under the appropriate excitatory spectra.

In one embodiment, following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, or another binding partner carrying a detectable moiety.

In particular embodiments, detection is by Southern blotting and hybridization with a labeled probe. The techniques involved in Southern blotting are well known to those of skill in the art. See Sambrook et al., 1989. One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

Other methods of nucleic acid detection that may be used in the practice of the instant invention are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference.

IX. Separation and Quantitation Methods

Following amplification, it may be desirable to separate the amplification products of several different lengths from each other and from the template and the excess primer for the purpose analysis or more specifically for determining whether specific amplification has occurred.

A. Gel Electrophoresis

In one embodiment, amplification products are separated by agarose, agarose-acrylamide or polyacrylamide gel electrophoresis using standard methods (Sambrook et al., 1989).

Separation by electrophoresis is based upon the differential migration through a gel according to the size and ionic charge of the molecules in an electrical field. High resolution techniques normally use a gel support for the fluid phase. Examples of gels used are starch, acrylamide, agarose or mixtures of acrylamide and agarose. Frictional resistance produced by the support causes size, rather than charge alone, to become the major determinant of separation. Smaller molecules with a more negative charge will travel faster and further through the gel toward the anode of an electrophoretic cell when high voltage is applied. Similar molecules will group on the gel. They may be visualized by staining and quantitated, in relative terms, using densitometers which continuously monitor the photometric density of the resulting stain. The electrolyte may be continuous. (a single buffer) or discontinuous, where a sample is stacked by means of a buffer discontinuity, before it enters the running gel/running buffer. The gel may be a single concentration or gradient in which pore size decreases with migration distance. In SDS gel electrophoresis of proteins or electrophoresis of polynucleotides, mobility depends primarily on size and is used to determined molecular weight. In pulse field electrophoresis, two fields are applied alternately at right angles to each other to minimize diffusion mediated spread of large linear polymers.

Agarose gel electrophoresis facilitates the separation of DNA or RNA based upon size in a matrix composed of a highly purified form of agar. Nucleic acids tend to become oriented in an end on position in the presence of an electric field. Migration through the gel matrices occurs at a rate inversely proportional to the log₁₀ of the number of base pairs (Sambrook et al., 1989).

Polyacrylamide gel electrophoresis (PAGE) is an analytical and separative technique in which molecules, particularly proteins, are separated by their different electrophoretic mobilities in a hydrated gel. The gel suppresses convective mixing of the fluid phase through which the electrophoresis takes place and contributes molecular sieving. Commonly carried out in the presence of the anionic detergent sodium dodecylsulphate (SDS). SDS denatures proteins so that noncovalently associating sub unit polypeptides migrate independently and by binding to the proteins confers a net negative charge roughly proportional to the chain weight.

B. Chromatographic Techniques

Alternatively, chromatographic techniques may be employed to effect separation. There are many kinds of chromatography which may be used in the present invention: adsorption, partition, ion-exchange and molecular sieve, and many specialized techniques for using them including column, paper, thin-layer and gas chromatography (Freifelder, 1982). In yet another alternative, labeled cDNA products, such as biotin or antigen can be captured with beads bearing avidin or antibody, respectively.

C. Microfluidic Techniques

Microfluidic techniques include separation on a platform such as microcapillaries, designed by ACLARA BioSciences Inc., or the LabChip™ “liquid integrated circuits” made by Caliper Technologies Inc. These microfluidic platforms require only nanoliter volumes of sample, in contrast to the microliter volumes required by other separation technologies. Miniaturizing some of the processes involved in genetic analysis has been achieved using microfluidic devices. For example, published PCT Application No. WO 94/05414, to Northrup and White, incorporated herein by reference, reports an integrated micro-PCR™ apparatus for collection and amplification of nucleic acids from a specimen. U.S. Pat. Nos. 5,304,487 and 5,296,375, discuss devices for collection and analysis of cell containing samples and are incorporated herein by reference. U.S. Pat. No. 5,856,174 describes an apparatus which combines the various processing and analytical operations involved in nucleic acid analysis and is incorporated herein by reference.

D. Capillary Electrophoresis

In some embodiments, it may be desirable to provide an additional, or alternative means for analyzing the amplified genes. In these embodiment, micro capillary arrays are contemplated to be used for the analysis.

Microcapillary array electrophoresis generally involves the use of a thin capillary or channel which may or may not be filled with a particular separation medium. Electrophoresis of a sample through the capillary provides a size based separation profile for the sample. The use of microcapillary electrophoresis in size separation of nucleic acids has been reported in, for example, Woolley and Mathies, 1994. Microcapillary array electrophoresis generally provides a rapid method for size-based sequencing, PCR™ product analysis and restriction fragment sizing. The high surface to volume ratio of these capillaries allows for the application of higher electric fields across the capillary without substantial thermal variation across the capillary, consequently allowing for more rapid separations. Furthermore, when combined with confocal imaging methods, these methods provide sensitivity in the range of attomoles, which is comparable to the sensitivity of radioactive sequencing methods. Microfabrication of microfluidic devices including microcapillary electrophoretic devices has been discussed in detail in, for example, Jacobsen et al., 1994; Effenhauser et al., 1994; Harrison et al., 1993; Effenhauser et al., 1993; Manz et al., 1992; and U.S. Pat. No. 5,904,824, here incorporated by reference. Typically, these methods comprise photolithographic etching of micron scale channels on a silica, silicon or other crystalline substrate or chip, and can be readily adapted for use in the present invention. In some embodiments, the capillary arrays may be fabricated from the same polymeric materials described for the fabrication of the body of the device, using the injection molding techniques described herein.

Tsuda et al., 1990, describes rectangular capillaries, an alternative to the cylindrical capillary glass tubes. Some advantages of these systems are their efficient heat dissipation due to the large height-to-width ratio and, hence, their high surface-to-volume ratio and their high detection sensitivity for optical on-column detection modes. These flat separation channels have the ability to perform two-dimensional separations, with one force being applied across the separation channel, and with the sample zones detected by the use of a multi-channel array detector.

In many capillary electrophoresis methods, the capillaries, e.g., fused silica capillaries or channels etched, machined or molded into planar substrates, are filled with an appropriate separation/sieving matrix. Typically, a variety of sieving matrices are known in the art may be used in the microcapillary arrays. Examples of such matrices include, e.g., hydroxyethyl cellulose, polyacrylamide, agarose and the like. Generally, the specific gel matrix, running buffers and running conditions are selected to maximize the separation characteristics of the particular application, e.g., the size of the nucleic acid fragments, the required resolution, and the presence of native or undenatured nucleic acid molecules. For example, running buffers may include denaturants, chaotropic agents such as urea or the like, to denature nucleic acids in the sample.

E. Mass Spectroscopy

Mass spectrometry provides a means of “weighing” individual molecules by ionizing the molecules in vacuo and making them “fly” by volatilization. Under the influence of combinations of electric and magnetic fields, the ions follow trajectories depending on their individual mass (m) and charge (z). For low molecular weight molecules, mass spectrometry has been part of the routine physical-organic repertoire for analysis and characterization of organic molecules by the determination of the mass of the parent molecular ion. In addition, by arranging collisions of this parent molecular ion with other particles (e.g., argon atoms), the molecular ion is fragmented forming secondary ions by the so-called collision induced dissociation (CID). The fragmentation pattern/pathway very often allows the derivation of detailed structural information. Other applications of mass spectrometric methods in the known in the art can be found summarized in Methods in Enzymology, Vol. 193: “Mass Spectrometry” (McCloskey, editor), 1990, Academic Press, New York.

Due to the apparent analytical advantages of mass spectrometry in providing high detection sensitivity, accuracy of mass measurements, detailed structural information by CID in conjunction with an MS/MS configuration and speed, as well as on-line data transfer to a computer, there has been considerable interest in the use of mass spectrometry for the structural analysis of nucleic acids. Reviews summarizing this field include Schram, 1990 and Crain, 1990 here incorporated by reference. The biggest hurdle to applying mass spectrometry to nucleic acids is the difficulty of volatilizing these very polar biopolymers. Therefore, “sequencing” had been limited to low molecular weight synthetic oligonucleotides by determining the mass of the parent molecular ion and through this, confirming the already known sequence, or alternatively, confirming the known sequence through the generation of secondary ions (fragment ions) via CID in an MS/MS configuration utilizing, in particular, for the ionization and volatilization, the method of fast atomic bombardment (FAB mass spectrometry) or plasma desorption (PD mass spectrometry). As an example, the application of FAB to the analysis of protected dimeric blocks for chemical synthesis of oligodeoxynucleotides has been described (Koster et al. 1987).

Two ionization/desorption techniques are electrospray/ionspray (ES) and matrix-assisted laser desorption/ionization (MALDI). ES mass spectrometry was introduced by Fenn, 1984; PCT Application No. WO 90/14148 and its applications are summarized in review articles, for example, Smith 1990 and Ardrey, 1992. As a mass analyzer, a quadrupole is most frequently used. The determination of molecular weights in femtomole amounts of sample is very accurate due to the presence of multiple ion peaks which all could be used for the mass calculation.

MALDI mass spectrometry, in contrast, can be particularly attractive when a time-of-flight (TOF) configuration is used as a mass analyzer. The MALDI-TOF mass spectrometry has been introduced by Hillenkamp 1990. Since, in most cases, no multiple molecular ion peaks are produced with this technique, the mass spectra, in principle, look simpler compared to ES mass spectrometry. DNA molecules up to a molecular weight of 410,000 daltons could be desorbed and volatilized (Williams, 1989). More recently, this the use of infra red lasers (IR) in this technique (as opposed to UV-lasers) has been shown to provide mass spectra of larger nucleic acids such as, synthetic DNA, restriction enzyme fragments of plasmid DNA, and RNA transcripts up to a size of 2180 nucleotides (Berkenkamp, 1998). Berkenkamp also describe how DNA and RNA samples can be analyzed by limited sample purification using MALDI-TOF IR.

In Japanese Patent No. 59-131909, an instrument is described which detects nucleic acid fragments separated either by electrophoresis, liquid chromatography or high speed gel filtration. Mass spectrometric detection is achieved by incorporating into the nucleic acids atoms which normally do not occur in DNA such as S, Br, I or Ag, Au, Pt, Os, Hg.

F. Energy Transfer

Labeling hybridization oligonucleotide probes with fluorescent labels is a well known technique in the art and is a sensitive, nonradioactive method for facilitating detection of probe hybridization. More recently developed detection methods employ the process of fluorescence energy transfer (FET) rather than direct detection of fluorescence intensity for detection of probe hybridization. FET occurs between a donor fluorophore and an acceptor dye (which may or may not be a fluorophore) when the absorption spectrum of one (the acceptor) overlaps the emission spectrum of the other (the donor) and the two dyes are in close proximity. Dyes with these properties are referred to as donor/acceptor dye pairs or energy transfer dye pairs. The excited-state energy of the donor fluorophore is transferred by a resonance dipole-induced dipole interaction to the neighboring acceptor. This results in quenching of donor fluorescence. In some cases, if the acceptor is also a fluorophore, the intensity of its fluorescence may be enhanced. The efficiency of energy transfer is highly dependent on the distance between the donor and acceptor, and equations predicting these relationships have been developed by Forster, 1948. The distance between donor and acceptor dyes at which energy transfer efficiency is 50% is referred to as the Forster distance (R_{O}). Other mechanisms of fluorescence quenching are also known including, for example, charge transfer and collisional quenching.

Energy transfer and other mechanisms which rely on the interaction of two dyes in close proximity to produce quenching are an attractive means for detecting or identifying nucleotide sequences, as such assays may be conducted in homogeneous formats. Homogeneous assay formats are simpler than conventional probe hybridization assays which rely on detection of the fluorescence of a single fluorophore label, as heterogeneous assays generally require additional steps to separate hybridized label from free label. Several formats for FET hybridization assays are reviewed in Nonisotopic DNA Probe Techniques (1992. Academic Press, Inc., pgs. 311-352).

Homogeneous methods employing energy transfer or other mechanisms of fluorescence quenching for detection of nucleic acid amplification have also been described. Higuchi (1992), discloses methods for detecting DNA amplification in real-time by monitoring increased fluorescence of ethidium bromide as it binds to double-stranded DNA. The sensitivity of this method is limited because binding of the ethidium bromide is not target specific and background amplification products are also detected. Lee, 1993, discloses a real-time detection method in which a doubly-labeled detector probe is cleaved in a target amplification-specific manner during PCR™. The detector probe is hybridized downstream of the amplification primer so that the 5′-3′ exonuclease activity of Taq polymerase digests the detector probe, separating two fluorescent dyes which form an energy transfer pair. Fluorescence intensity increases as the probe is cleaved. Published PCT application WO 96/21144 discloses continuous fluorometric assays in which enzyme-mediated cleavage of nucleic acids results in increased fluorescence. Fluorescence energy transfer is suggested for use in the methods, but only in the context of a method employing a single fluorescent label which is quenched by hybridization to the target.

Signal primers or detector probes which hybridize to the target sequence downstream of the hybridization site of the amplification primers have been described for use in detection of nucleic acid amplification (U.S. Pat. No. 5,547,861). The signal primer is extended by the polymerase in a manner similar to extension of the amplification primers. Extension of the amplification primer displaces the extension product of the signal primer in a target amplification-dependent manner, producing a double-stranded secondary amplification product which may be detected as an indication of target amplification. The secondary amplification products generated from signal primers may be detected by means of a variety of labels and reporter groups, restriction sites in the signal primer which are cleaved to produce fragments of a characteristic size, capture groups, and structural features such as triple helices and recognition sites for double-stranded DNA binding proteins.

Many donor/acceptor dye pairs known in the art and may be used in the present invention. These include, for example, fluorescein isothiocyanate (FITC)/tetramethylrhodamine isothiocyanate (TRITC), FITC/Texas Red™. (Molecular Probes), FITC/N-hydroxysuccinimidyl 1-pyrenebutyrate (PYB), FITC/eosin isothiocyanate (EITC), N-hydroxysuccinimidyl 1-pyrenesulfonate (PYS)/FITC, FITC/Rhodamine X, FITC/tetramethylrhodamine (TAMRA), and others. The selection of a particular donor/acceptor fluorophore pair is not critical. For energy transfer quenching mechanisms it is only necessary that the emission wavelengths of the donor fluorophore overlap the excitation wavelengths of the acceptor, i.e., there must be sufficient spectral overlap between the two dyes to allow efficient energy transfer, charge transfer or fluorescence quenching. P-(dimethyl aminophenylazo)benzoic acid (DABCYL) is a non-fluorescent acceptor dye which effectively quenches fluorescence from an adjacent fluorophore, e.g., fluorescein or 5-(2′-aminoethyl)aminonaphthalene (EDANS). Any dye pair which produces fluorescence quenching in the detector nucleic acids of the invention are suitable for use in the methods of the invention, regardless of the mechanism by which quenching occurs. Terminal and internal labeling methods are both known in the art and maybe routinely used to link the donor and acceptor dyes at their respective sites in the detector nucleic acid.

G. Chip Technologies

DNA arrays and gene chip technology provides a means of rapidly screening a large number of DNA samples for their ability to hybridize to a variety of single stranded DNA probes immobilized on a solid substrate. Specifically contemplated are chip-based DNA technologies such as those described by Hacia et al., (1996) and Shoemaker et al. (1996). These techniques involve quantitative methods for analyzing large numbers of genes rapidly and accurately The technology capitalizes on the complementary binding properties of single stranded DNA to screen DNA samples by hybridization. Pease et al., 1994; Fodor et al., 1991. Basically, a DNA array or gene chip consists of a solid substrate upon which an array of single stranded DNA molecules have been attached. For screening, the chip or array is contacted with a single stranded DNA sample which is allowed to hybridize under stringent conditions. The chip or array is then scanned to determine which probes have hybridized. In the context of this embodiment, such probes could include synthesized oligonucleotides, cDNA, genomic DNA, yeast artificial chromosomes (YACs), bacterial artificial chromosomes (BACs), chromosomal markers or other constructs a person of ordinary skill would recognize as adequate to demonstrate a genetic change.

A variety of gene chip or DNA array formats are described in the art, for example U.S. Pat. Nos. 5,861,242 and 5,578,832 which are expressly incorporated herein by reference. A means for applying the disclosed methods to the construction of such a chip or array would be clear to one of ordinary skill in the art. In brief, the basic structure of a gene chip or array comprises: (1) an excitation source; (2) an array of probes; (3) a sampling element; (4) a detector; and (5) a signal amplification/treatment system. A chip may also include a support for immobilizing the probe.

In particular embodiments, a target nucleic acid may be tagged or labeled with a substance that emits a detectable signal; for example, luminescence. The target nucleic acid may be immobilized onto the integrated microchip that also supports a phototransducer and related detection circuitry. Alternatively, a gene probe may be immobilized onto a membrane or filter which is then attached to the microchip or to the detector surface itself. In a further embodiment, the immobilized probe may be tagged or labeled with a substance that emits a detectable or altered signal when combined with the target nucleic acid. The tagged or labeled species may be fluorescent, phosphorescent, or otherwise luminescent, or it may emit Raman energy or it may absorb energy. When the probes selectively bind to a targeted species, a signal is generated that is detected by the chip. The signal may then be processed in several ways, depending on the nature of the signal.

The DNA probes may be directly or indirectly immobilized onto a transducer detection surface to ensure optimal contact and maximum detection. The ability to directly synthesize on or attach polynucleotide probes to solid substrates is well known in the art. See U.S. Pat. Nos. 5,837,832 and 5,837,860 both of which are expressly incorporated by reference. A variety of methods have been utilized to either permanently or removably attach the probes to the substrate. Exemplary methods include: the immobilization of biotinylated nucleic acid molecules to avidin/streptavidin coated supports (Holmstrom, 1993), the direct covalent attachment of short, 5′-phosphorylated primers to chemically modified polystyrene plates (Rasmussen, et al., 1991), or the precoating of the polystyrene or glass solid phases with poly-L-Lys or poly L-Lys, Phe, followed by the covalent attachment of either amino- or sulfhydryl-modified oligonucleotides using bi-functional crosslinking reagents. (Running, et al., 1990); Newton, et al. (1993)). When immobilized onto a substrate, the probes are stabilized and therefore may be used repeatedly. In general terms, hybridization is performed on an immobilized nucleic acid target or a probe molecule is attached to a solid surface such as nitrocellulose, nylon membrane or glass. Numerous other matrix materials may be used, including reinforced nitrocellulose membrane, activated quartz, activated glass, polyvinylidene difluoride (PVDF) membrane, polystyrene substrates, polyacrylamide-based substrate, other polymers such as poly(vinyl chloride), poly(methyl methacrylate), poly(dimethyl siloxane), photopolymers (which contain photoreactive species such as nitrenes, carbenes and ketyl radicals capable of forming covalent links with target molecules.

Binding of the probe to a selected support may be accomplished by any of several means. For example, DNA is commonly bound to glass by first silanizing the glass surface, then activating with carbodimide or glutaraldehyde. Alternative procedures may use reagents such as 3-glycidoxypropyltrimethoxysilane (GOP) or aminopropyltrimethoxysilane (APTS) with DNA linked via amino linkers incorporated either at the 3′ or 5′ end of the molecule during DNA synthesis. DNA may be bound directly to membranes using ultraviolet radiation. With nitrocellous membranes, the DNA probes are spotted onto the membranes. A UV light source (Stratalinker, from Stratagene, La Jolla, Calif.) is used to irradiate DNA spots and induce cross-linking. An alternative method for cross-linking involves baking the spotted membranes at 80° C. for two hours in vacuum.

Specific DNA probes may first be immobilized onto a membrane and then attached to a membrane in contact with a transducer detection surface. This method avoids binding the probe onto the transducer and may be desirable for large-scale production. Membranes particularly suitable for this application include nitrocellulose membrane (e.g., from BioRad, Hercules, Calif.) or polyvinylidene difluoride (PVDF). (BioRad, Hercules, Calif.) or nylon membrane (Zeta-Probe, BioRad) or polystyrene base substrates (DNA.BIND™ Costar, Cambridge, Mass.).

X. Identification Methods

Amplification products must be visualized in order to confirm amplification of the target-gene(s) sequences. One typical visualization method involves staining of a gel with for example, a fluorescent dye, such as ethidium bromide or Vista Green and visualization under UV light. Alternatively, if the amplification products are integrally labeled with radio- or fluorometrically-labeled nucleotides, the amplification products can then be exposed to x-ray film or visualized under the appropriate stimulating spectra, following separation.

In one embodiment, visualization is achieved indirectly, using a nucleic acid probe. Following separation of amplification products, a labeled, nucleic acid probe is brought into contact with the amplified gene(s) sequence. The probe preferably is conjugated to a chromophore but may be radiolabeled. In another embodiment, the probe is conjugated to a binding partner, such as an antibody or biotin, where the other member of the binding pair carries a detectable moiety. In other embodiments, the probe incorporates a fluorescent dye or label. In yet other embodiments, the probe has a mass label that can be used to detect the molecule amplified. Other embodiments also contemplate the use of Taqman™ and Molecular Beacon™ probes. In still other embodiments, solid-phase capture methods combined with a standard probe may be used as well.

The type of label incorporated in PCR™ products is dictated by the method used for analysis. When using capillary electrophoresis, microfluidic electrophoresis, HPLC, or LC separations, either incorporated or intercalated fluorescent dyes are used to label and detect the PCR™ products. Samples are detected dynamically, in that fluorescence is quantitated as a labeled species moves past the detector. If any electrophoretic method, HPLC, or LC is used for separation, products can be detected by absorption of UV light, a property inherent to DNA and therefore not requiring addition of a label. If polyacrylamide gel or slab gel electrophoresis is used, primers for the PCR™ can be labeled with a fluorophore, a chromophore or a radioisotope, or by associated enzymatic reaction. Enzymatic detection involves binding an enzyme to primer, e.g., via a biotin:avidin interaction, following separation of PCR™ products on a gel, then detection by chemical reaction, such as chemiluminescence generated with luminol. A fluorescent signal can be monitored dynamically. Detection with a radioisotope or enzymatic reaction requires an initial separation by gel electrophoresis, followed by transfer of DNA molecules to a solid support (blot) prior to analysis. If blots are made, they can be analyzed more than once by probing, stripping the blot, and then reprobing. If PCR™ products are separated using a mass spectrometer no label is required because nucleic acids are detected directly.

A number of the above separation platforms can be coupled to achieve separations based on two different properties. For example, some of the PCR™ primers can be coupled with a moiety that allows affinity capture, and some primers remain unmodified. Modifications can include a sugar (for binding to a lectin column), a hydrophobic group (for binding to a reverse-phase column), biotin (for binding to a streptavidin column), or an antigen (for binding to an antibody column). Samples are run through an affinity chromatography column. The flow-through fraction is collected, and the bound fraction eluted (by chemical cleavage, salt elution, etc.). Each sample is then further fractionated based on a property, such as mass, to identify individual components.

XI. Sequencing

It is envisioned that amplified product will commonly be sequenced for further identification. Sanger dideoxy-termination sequencing is the means commonly employed to determine nucleotide sequence. The Sanger method employs a short oligonucleotide or primer that is annealed to a single-stranded template containing the DNA to be sequenced. The primer provides a 3′ hydroxyl group which allows the polymerization of a chain of DNA when a polymerase enzyme and dNTPs are provided. The Sanger method is an enzymatic reaction that utilizes chain-terminating dideoxynucleotides (ddNTPs). ddNTPs are chain-terminating because they lack a 3′-hydroxyl residue which prevents formation of a phosphodiester bond with a succeeding deoxyribonucleotide (dNTP). A small amount of one ddNTP is included with the four conventional dNTPs in a polymerization reaction. Polymerization or DNA synthesis is catalyzed by a DNA polymerase. There is competition between extension of the chain by incorporation of the conventional dNTPs and termination of the chain by incorporation of a ddNTP.

Although a variety of polymerases may be used, the use of a modified T7 DNA polymerase (Sequenase™) was a significant improvement over the original Sanger method (Sambrook et al., 1988; Hunkapiller, 1991). T7 DNA polymerase does not have any inherent 5′-3′ exonuclease activity and has a reduced selectivity against incorporation of ddNTP. However, the 3′-5′ exonuclease activity leads to degradation of some of the oligonucleotide primers. Sequenase™ is a chemically-modified T7 DNA polymerase that has reduced 3′ to 5′ exonuclease activity (Tabor et al., 1987). Sequenase™ version 2.0 is a genetically engineered form of the T7 polymerase which completely lacks 3′ to 5′ exonuclease activity. Sequenase™ has a very high processivity and high rate of polymerization. It can efficiently incorporate nucleotide analogs such as dITP and 7-deaza-dGTP which are used to resolve regions of compression in sequencing gels. In regions of DNA containing a high G+C content, Hoogsteen bond formation can occur which leads to compressions in the DNA. These compressions result in aberrant migration patterns of oligonucleotide strands on sequencing gels. Because these base analogs pair weakly with conventional nucleotides, intrastrand secondary structures during electrophoresis are alleviated. In contrast, Klenow does not incorporate these analogs as efficiently.

The use of Taq DNA polymerase and mutants thereof is a more recent addition to the improvements of the Sanger method (U.S. Pat. No. 5,075,216). Taq polymerase is a thermostable enzyme which works efficiently at 70-75° C. The ability to catalyze DNA synthesis at elevated temperature makes Taq polymerase useful for sequencing templates which have extensive secondary structures at 37° C. (the standard temperature used for Klenow and Sequenase™ reactions). Taq polymerase, like Sequenase™, has a high degree of processivity and like Sequenase 2.0, it lacks 3′ to 5′ nuclease activity. The thermal stability of Taq and related enzymes (such as Tth and Thermosequenase™) provides an advantage over T7 polymerase (and all mutants thereof) in that these thermally stable enzymes can be used for cycle sequencing which amplifies the DNA during the sequencing reaction, thus allowing sequencing to be performed on smaller amounts of DNA. Optimization of the use of Taq in the standard Sanger Method has focused on modifying Taq to eliminate the intrinsic 5′-3′ exonuclease activity and to increase its ability to incorporate ddNTPs to reduce incorrect termination due to secondary structure in the single-stranded template DNA (EP 0 655 506 B1). The introduction of fluorescently labeled nucleotides has further allowed the introduction of automated sequencing which further increases processivity.

XII. DNA Immobilization

Immobilization of the DNA may be achieved by a variety of methods involving either non-covalent or covalent interactions between the immobilized DNA comprising an anchorable moiety and an anchor. In a preferred embodiment of the invention, immobilization consists of the non-covalent coating of a solid phase with streptavidin or avidin and the subsequent immobilization of a biotinylated polynucleotide (Holmstrom, 1993). It is further envisioned that immobilization may occur by precoating a polystyrene or glass solid phase with poly-L-Lys or poly L-Lys, Phe, followed by the covalent attachment of either amino- or sulfhydryl-modified polynucleotides using bifunctional crosslinking reagents (Running, 1990 and Newton, 1993).

Immobilization may also take place by the direct covalent attachment of short, 5′-phosphorylated primers to chemically modified polystyrene plates (“Covalink” plates, Nunc) Rasmussen, (1991). The covalent bond between the modified oligonucleotide and the solid phase surface is introduced by condensation with a water-soluble carbodiimide. This method facilitates a predominantly 5′-attachment of the oligonucleotides via their 5′-phosphates.

Nikiforov et al. (U.S. Pat. No. 5,610,287 incorporated herein by reference) describes a method of non-covalently immobilizing nucleic acid molecules in the presence of a salt or cationic detergent on a hydrophilic polystyrene solid support containing a hydrophilic moiety or on a glass solid support. The support is contacted with a solution having a pH of about 6 to about 8 containing the synthetic nucleic acid and a cationic detergent or salt. The support containing the immobilized nucleic acid may be washed with an aqueous solution containing a non-ionic detergent without removing the attached molecules.

Another commercially available method envisioned by the inventors to facilitate immobilization is the “Reacti-Bind.™. DNA Coating Solutions” (see “Instructions—Reacti-Bind.™. DNA Coating Solution” 1/1997). This product comprises a solution that is mixed with DNA and applied to surfaces such as polystyrene or polypropylene. After overnight incubation, the solution is removed, the surface washed with buffer and dried, after which it is ready for hybridization. It is envisioned that similar products, i.e. Costar “DNA-BIND™” or Immobilon-AV Affinity Membrane (IAV, Millipore, Bedford, Mass.) are equally applicable to immobilize the respective fragment.

XIII. Analysis of Data

Gathering data from the various analysis operations will typically be carried out using methods known in the art. For example, microcapillary arrays may be scanned using lasers to excite fluorescently labeled targets that have hybridized to regions of probe arrays, which can then be imaged using charged coupled devices (“CCDs”) for a wide field scanning of the array. Alternatively, another particularly useful method for gathering data from the arrays is through the use of laser confocal microscopy which combines the ease and speed of a readily automated process with high resolution detection. Scanning devices of this kind are described in U.S. Pat. Nos. 5,143,854 and 5,424,186.

Following the data gathering operation, the data will typically be reported to a data analysis operation. To facilitate the sample analysis operation, the data obtained by a reader from the device will typically be analyzed using a digital computer. Typically, the computer will be appropriately programmed for receipt and storage of the data from the device, as well as for analysis and reporting of the data gathered, i.e., interpreting fluorescence data to determine the sequence of hybridizing probes, normalization of background and single base mismatch hybridizations, ordering of sequence data in SBH applications, and the like, as described in, e.g., U.S. Pat. Nos. 4,683,194; 5,599,668; and 5,843,651, each of which is incorporated herein by reference.

XIV. PENTAmer Libraries as a Resource for Highly Multiplexed DNA Amplification

PENTAmer technology creates a new paradigm for DNA handling including a better solution for high throughput SNP analysis. By parallel amplification of thousands of DNA samples, the PENTAmer technology solves the bottleneck problem of many current approaches and facilitates the development of new methods for SNP detection.

In general, two types of PENTAmers (Primer Extension Nick Translation Amplimers) are proposed: primary PENTAmers and recombinant PENTAmers.

Primary PENTAmers

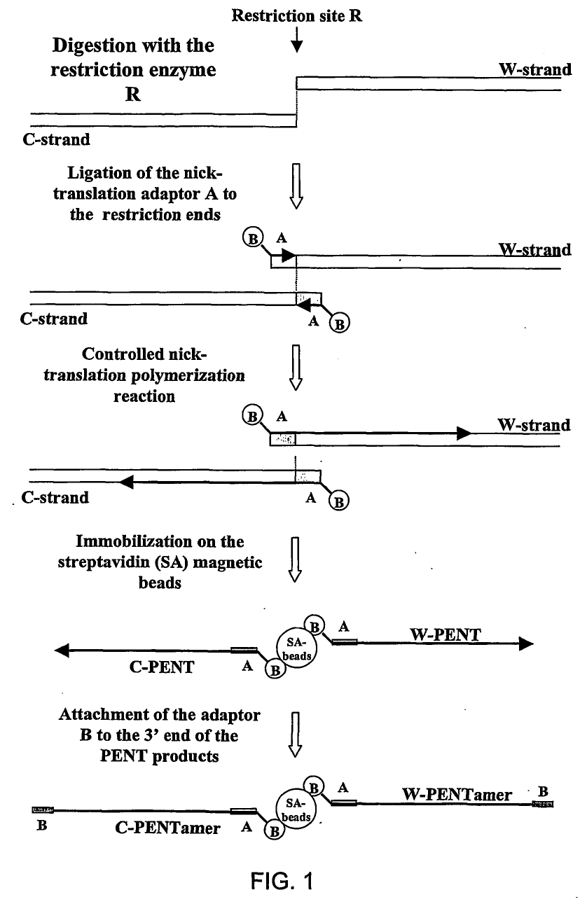

Primary PENTAmers represent a library of single-stranded DNA molecules of a similar size (i.e. 1 kb), which are produced by a controlled nick-translation polymerization reaction from the ends of DNA restriction fragments, FIG. 1. The 5′ “restriction” end of the primary PENTAmer begins at the restriction cleavage site, and it is linked to the nick-translation adaptor sequence A. The 3′ “fuzzy” end of the PENTAmer terminates with the internal nick-attaching adaptor B. Each restriction site gives rise to the two PENTAmer molecules: W-PENTAmer and C-PENTAmer, produced by the replacement synthesis of the original W and C strands of a double stranded DNA, respectively (FIG. 1). The obvious advantages of using PENTAmers for DNA amplification are the universal size and universal adaptor sequences A and B at the ends of all DNA amplicons.

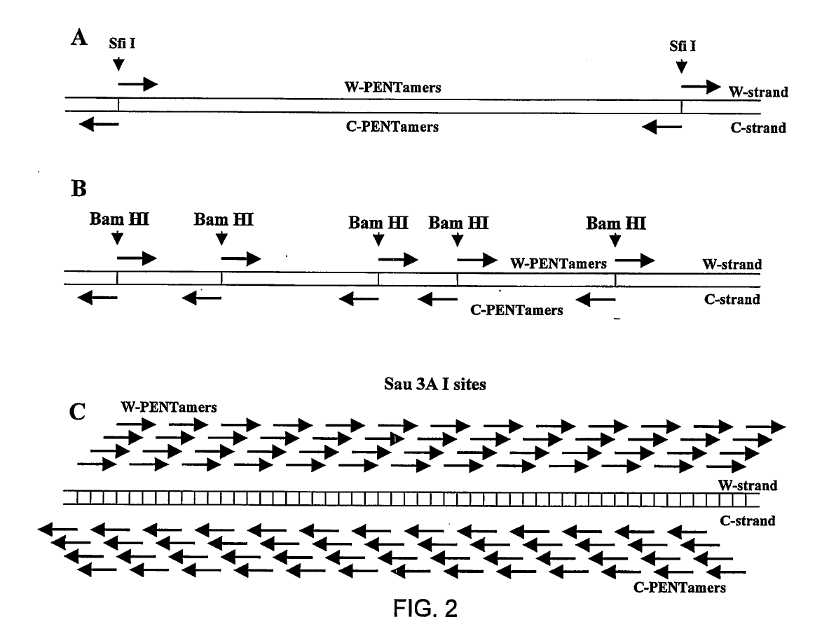

Depending on the type and mode of the restriction endonuclease cleavage, the PENTAmer libraries might represent the whole genome or only part of it. For example, complete digestion of human DNA with the Sfi I restriction endonuclease produces non-overlapping DNA fragments of 100 kb average size (FIG. 2A). In this first case, 1 kb PENTAmer library would represent about 1/50 or 2% non-redundant coverage of the whole genome and allow one to genotype DNA with a density of about 1 SNP per 50 kb, assuming a generally accepted occurrence of 1 SNP/kb.

Complete digestion of human DNA with the Bam H I restriction endonuclease produces non-overlapping DNA fragments of 12 kb average size (FIG. 2B). In this second case, 1 kb PENTAmer library would represent about ⅙ or 17% non-redundant coverage of the whole genome and allow one to genotype DNA with a density of about 1 SNP per 6 kb. Partial digestion of DNA with frequently cutting endonuclease Sau3A I allows one to synthesize a different type of PENTAmer library (FIG. 2C). In this case, the library is redundant, and it contains an average of 4 overlapping (1 kb) PENTAmer fragments per 1 kb of genomic DNA.

Narrow size distribution and universal adaptor sequences at the ends of the PENTAmer amplicons allows essentially unbiased amplification (linear or exponential) of the whole library or of the specific parts of the library.

Understanding genetic variations and association of polymorphisms with disease requires analysis of substantial number of SNPs (10⁵-10⁶) within a large population group (10³-10⁵). Thus, total number of polymorphisms to analyze is tremendous (10⁸-10¹¹), and it can be only achieved by high throughput parallel analysis of multiple DNA samples. Two practical aspects complicate the analysis:

High throughput parallel analysis of multiple loci in many DNA samples can be achieved by using PENTAmer libraries and two ways of multiplexing the amplification process. If one assumes that it is necessary to analyze m SNPs from p individuals, then the total number of SNPs to screen N=s×p. For example, if s=200,000 and p=1000, total number of SNP to analyze N=2×10⁸.

XV. Multiplexed Amplification of PENTAmers with Different Genomic Content but Originated from the Same PENTAmer Library

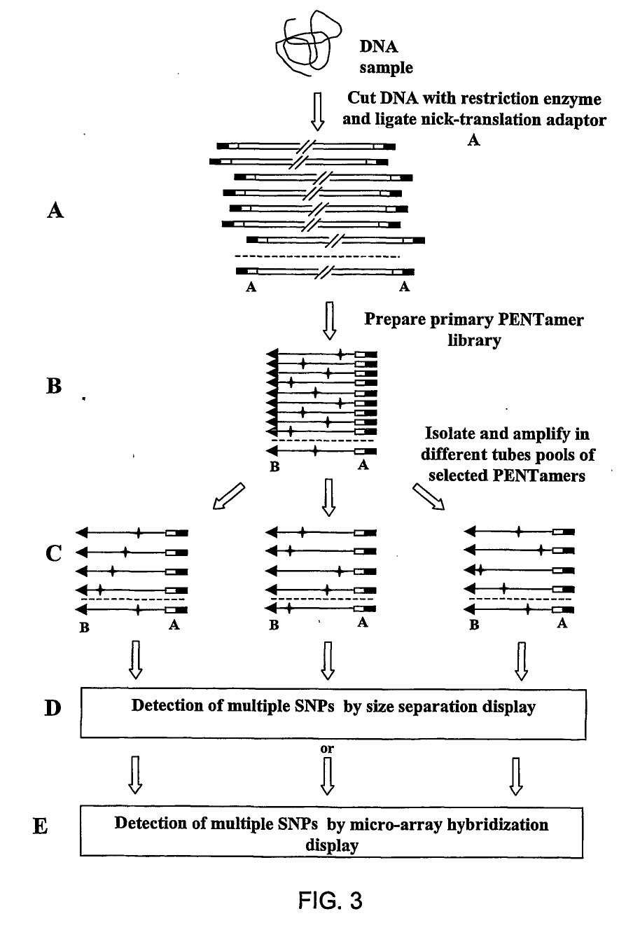

In the first approach, shown in FIG. 3, the multiplexing is achieved by a parallel amplification of many different SNP-containing PENTAmer amplicons within only one DNA sample (genome-wide multiplexing). In this case, only one nick-translation adaptor A is necessary. The SNP multiplex index m can vary from 2 to 1000 depending on other parameters.

XVI. Multiplexed Amplification of PENTAmer with the Same Genomic Content but Originated from Different PENTAmer Libraries

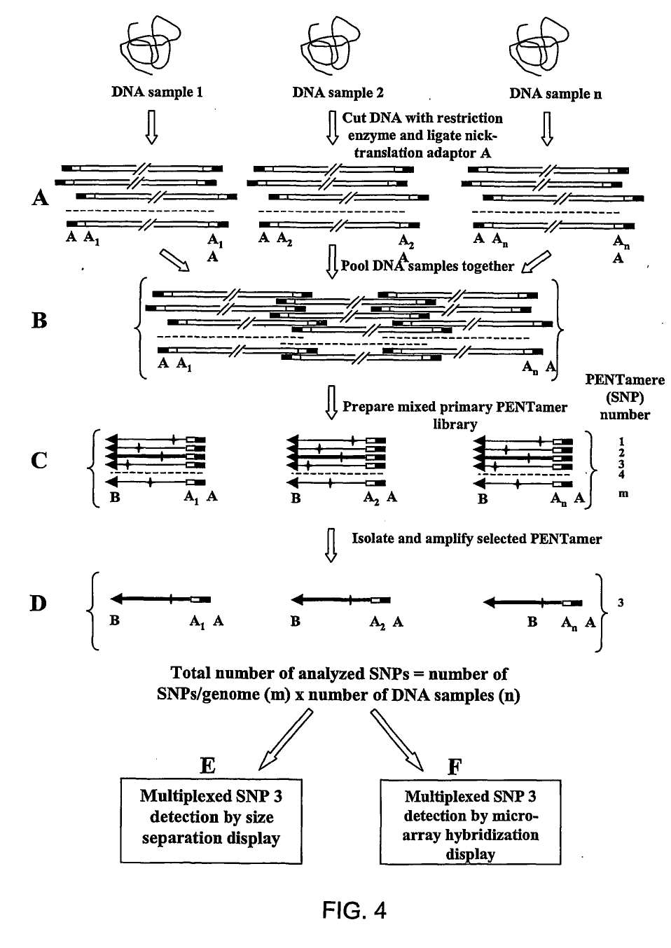

In the second approach, shown in FIG. 4, the multiplexing is achieved by a parallel amplification of only one SNP-containing PENTAmer amplicon within many different patient DNA samples (sample-wide multiplexing).

Two enzymatic steps are performed individually with every sample prior the multiplexing:

1. Digestion with a restriction enzyme (complete or partial)

2. Ligation of the library-specific nick-translation adaptor An.

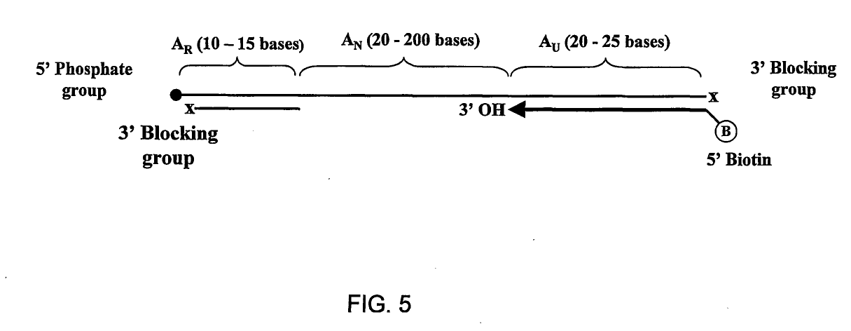

The set of n different nick-translation adaptors ALS (n=1, 2 . . . , n) used in this approach have two universal sequences AU and AR located distal and proximal to the restriction site, respectively (FIG. 5). The universal part AU of all adaptors is used to prime the nick-translation reaction, to capture the primary PENTAmer molecule on the streptavidin magnetic beads, and to prime the library amplification process. The universal part AR of all adaptors is used to direct the ligation of the adaptors to the ends of DNA restriction fragments.

Internal library-specific variable parts AN of the nick-translation adaptor ALS can have the same size but different base composition (sequence tags), the same sequence motif, but different length (length tags), or different sequence and length (general tags) (FIG. 5). The sample multiplex index n can vary from 2 to 1000 depending on the other parameters (for example, SNP multiplex index).

Protocol for the preparation of multi-patient PENTAmer library.

1. Digest n DNA samples isolated from n patients separately with restriction enzyme R (completely or partially). Heat-inactivate the restriction enzyme.

2. Adjust buffer conditions and incubate digested DNA samples with thermo-sensitive alkaline phosphotase (AP). Heat inactivate the AP. Purify the DNA samples by phenol/chlorophorm extraction/Ethanol precipitation or any other way, if necessary, for the next step.

3. Adjust buffer conditions and incubate n DNA samples after AP treatment with T4 DNA ligase and n different library-specific nick-translation adaptors ALS.

4. Mix n DNA samples together in one tube. Purify the DNA.

5. Adjust the buffer conditions and incubate for a specific time with Taq DNA polymerase (wild type) to produce the nick-translate (PENT) products.

6. Isolate the nick-translate products by affinity capture using the streptavidin-coated magnetic beads. Wash the products with NaOH, then with the ligation buffer.

7. Ligate the second adaptor B to the 3′ ends of the PENT products. Wash with NaOH, then with TE buffer. At this point, the preparation of the multi-patient primary PENTAmer library is completed.

8. Aliquot the library into a micro plate and amplify using universal primers B or A and B, appropriate polymerase and conditions, and linear or exponential mode, correspondingly.

Both approaches allow a high throughput genome-wide genotyping of SNP for large number of patient DNA samples. For example, if the sample multiplex index in the first approach n=100, the total number of SNP to analyze is reduced from N=2×10⁸ to N/n=2×10⁶. Similar, if the SNP multiplex index in the second approach m=100, the total number of reactions to analyze is again reduced from N=2×10⁸ to N/m=2×10⁶.

A combined multiplexing strategy with both sample multiplex index n and SNP multiplex index m are >2 can also be used. In this case, the combined multiplex index is determined by a factor m×n. For example, if number of mixed patient DNA samples n=50 and number of simultaneously amplified different SNPs m=10 the combined multiplex index m×n=50×10=500 and the total number of reactions to analyze would be reduced from N=2×10⁸to N/500=4×10⁵.

XVII. Whole PENTAmer Library Amplification as a Means to Generate DNA for High Throughput Multiple-Loci Genotyping and Diagnostics

There is an increasing demand in analyzing small amounts of DNA from limited quantities of tissue. Whenever the number of tests is very high, as in the case of whole-genome SNP scoring, or the amount of available material is small, as in the case of diagnostics of needle biopsies, the PENTAmer technology provides a universal solution to the problem.

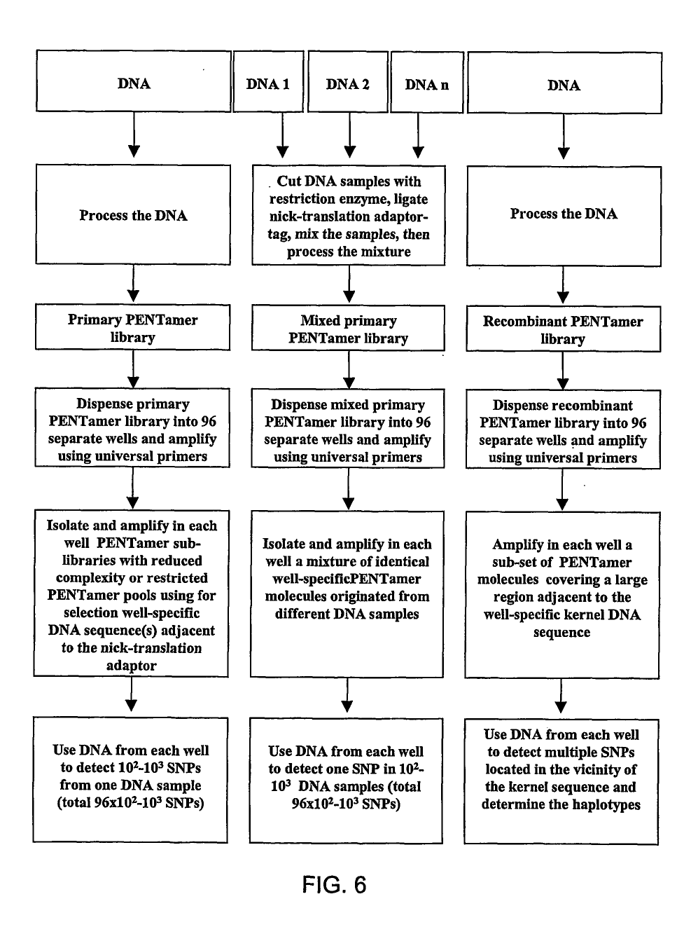

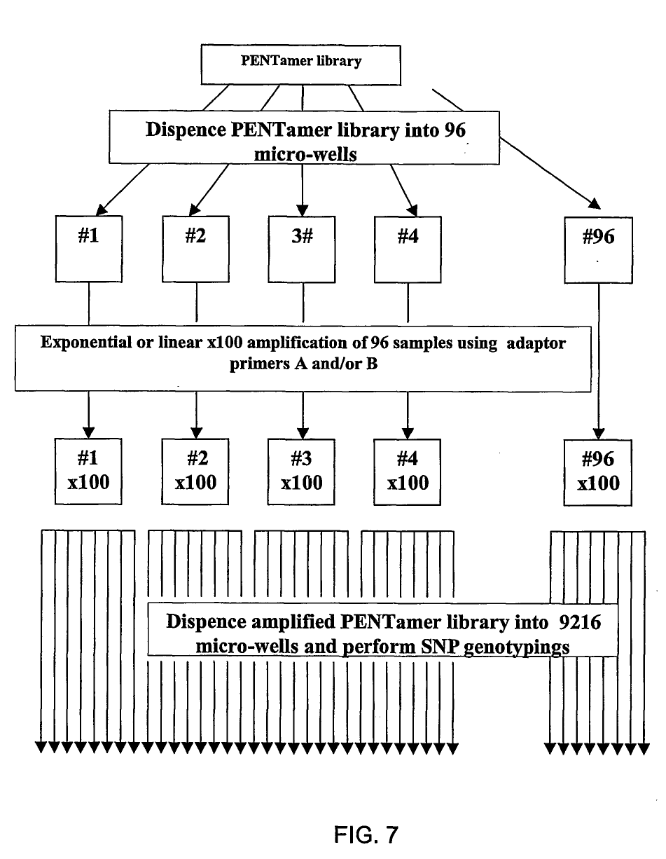

All three types of PENTAmer libraries, namely, (a) primary PENTAmer library prepared from one individual, (b) mixed primary PENTAmer library prepared from many different individuals, and (c) recombinant PENTAmer library (usually prepared from one individual) can be amplified using universal adaptor sequences attached to the ends of PENTAmers (FIG. 6 and FIG. 7).

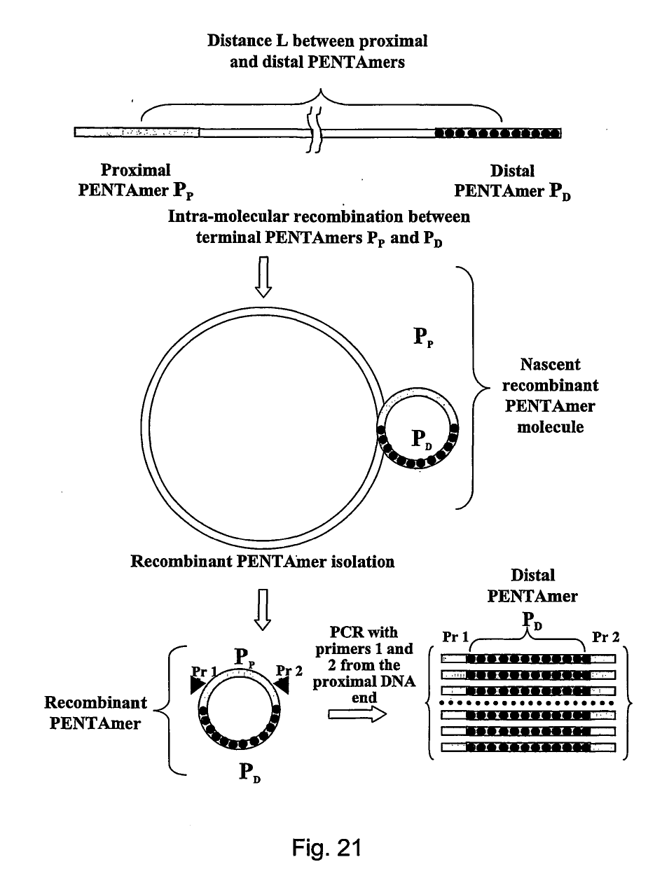

The amplification can be performed in an exponential or linear mode. In the exponential PCR mode two primers are used. In the case of primary PENTAmer library, the two primers are complementary to the adaptor A and B (FIG. 1). In the case when several PENTAmer libraries are pooled together, one of the primers is complementary to the external universal part AU of the modified adaptor ALS (FIG. 4 and FIG. 5). The second primer is complementary to the adaptor B sequence. The recombinant PENTAmer library is amplified using primers complementary to adaptor sequences located at the ends of recombinant molecules FIG. 21.

During PCR mode, the number of DNA amplicons within the library is doubled every cycle, so following 10 cycles the number of PENTAmers can be increased up to 1000 times, providing DNA is sufficient for at least 200,000 single genotyping experiments.

Linear amplification is performed with just one of primers used in the PCR mode.

XVIII. Primary PENTAmer Library as a Tool for Highly Multiplexed Selection and Amplification of DNA for Whole-Genome SNP Genotyping

In an object of the present invention, a primary PENTAmer library is efficiently implemented for a highly multiplexed selection and amplification of multiple DNA regions to allow a cost effective whole-genome SNP analysis.

A primary PENTAmer library can be generated with various degrees of complexity and coverage (FIG. 2). The complexity of the PENTAmer library depends on the frequency of DNA cleavage by a restriction enzyme used for the library preparation (FIG. 2). For example, human library produced by Sfi I restriction endonuclease is expected to have 60,000, library produced by BamH I restriction endonuclease—500,000, and library prepared after partial digestion with Sau3A I restriction endonuclease—more than 25 million different PENTAmers. This section describes the isolation of specific PENTAmers from a primary PENTAmer library and the subdivision of a primary PENTAmer library into specific pools for the purposes of multiplexed SNP detection.

Specific DNA sequences within the primary library can be systematically isolated either individually or in combination. Isolation of a specific PENTAmer is described in Examples 1, 4 and 7. The procedure can also be used in a multiplexed format; necessary modifications are described in Examples 2, 5 and 8. Examples 3, 6 and 9 describe how specialized selector oligonucleotides are used to segregate entire PENTAmer libraries into particular pools. Examples 1, 2 and 3 utilize the ligation-mediated capture protocols. Examples 4, 5 and 6 are based on the polymerization-mediated capture procedure. Examples 7, 8 and 9 use PCR amplification protocols.

A. Isolation of Specific PENTAmers and Subdivision of PENTAmer Libraries by Ligation-Mediated Capture

This section describes the isolation of specific PENTAmers from a primary PENTAmer library and subdivision of a primary PENTAmer library into specific pools using ligation-mediated capture procedure. A unique hairpin oligonucleotide and a specific selective oligonucleotide are covalently attached to the PENTAmer(s) of interest by the enzyme DNA ligase. The selective oligonucleotide is designed with an affinity tag that permits capture of the target molecules. Specific capture permits the analysis of unique DNA molecules. Subdivision of the library allows reduction in the complexity of the subsequent pools. Captured molecules can be examined directly or amplified and re-selected to enrich the products.

The following is an illustration of preferred embodiments for practicing the present invention. However, they are not limiting examples. Other examples and methods are possible in practicing the present invention.

### EXAMPLE 1

### Specific Primary Pentamer Isolation by 5′ End Ligation-Mediated Capture

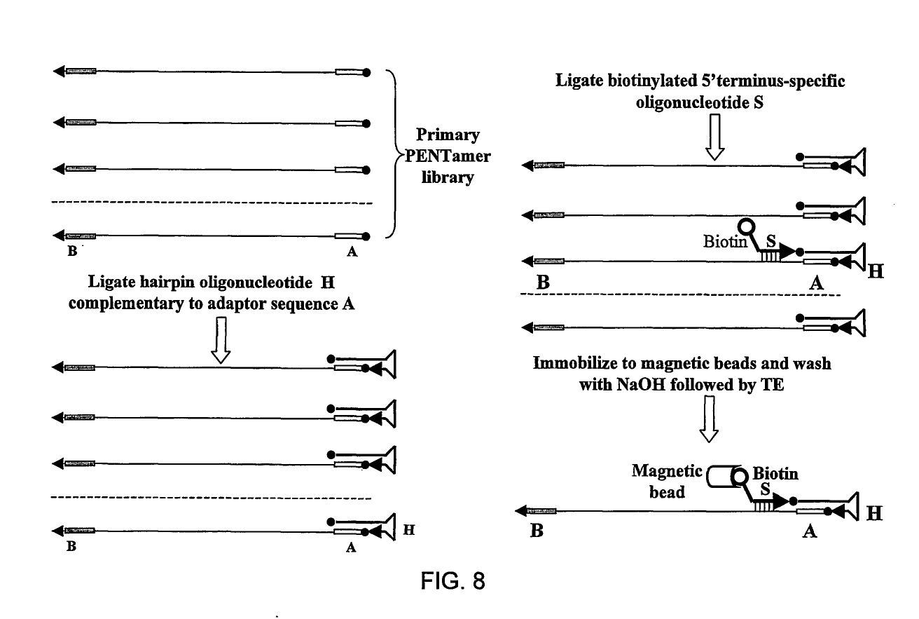

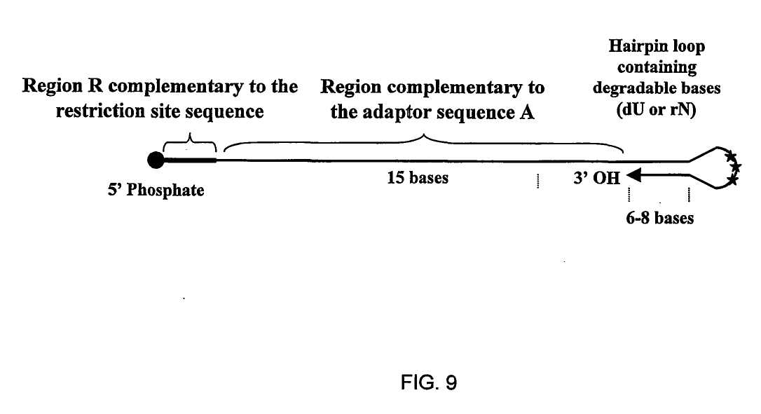

The first step in isolation of a specific PENTAmer is the ligation of the hairpin oligonucleotide H (FIGS. 8A and 8B). The hairpin oligonucleotide is complementary to adaptor A of the PENTAmer library (FIG. 9), to enable annealing and ligation to all molecules in the PENTAmer library. This step relies on simple base pairing and subsequent ligation using standard DNA ligase conditions. For example, T4 DNA ligase as Tsc thermostable ligase could be used in conjunction with the corresponding manufacturer protocols.

There are several features important to the function of the hairpin oligonucleotide H (FIG. 9). It must contain a 3′ OH terminus to accommodate ligation of the 5′ phosphate from adaptor A of the PENTAmer library. The 3′ OH terminus is preceded by a short double-stranded stretch containing the hairpin or loop region. This loop can be of various sizes to accommodate the structural turn necessary for the intramolecular annealing of the hairpin. It can contain labile bases, such as deoxyuridine or ribonucleotides or other, which can be enzymatically (or chemically) degraded to release the ligated PENTAmers at later steps. These or other specialized bases can be incorporated during the chemical synthesis of the hairpin oligonucleotide. The hairpin oligonucleotide also contains a region complementary to adaptor A for annealing and alignment of the hairpin loop 3′ OH with the 5′ phosphate of adaptor A. Extent of complementarity is dependent on the length of adaptor A (in FIG. 9, it is shown as 25 bases) but should change in proportion to any changes made in adaptor A. Region R is complementary to the restriction site sequence used in the PENTAmer library construction. Lastly, the 5′ terminus of the hairpin oligonucleotide H is phosphorylated. The phosphate is necessary for ligation of a selector-capture oligonucleotide.

Once the hairpin oligonucleotide H is attached, a sequence specific selector-capture oligonucleotide is annealed to the PENTAmer library. The sequence is complementary to known DNA sequence adjacent to the paired adaptor A and hairpin oligonucleotide H. Incubation with DNA ligase will covalently join only selector-capture oligonucleotides annealed immediately adjacent to the paired adaptor A and hairpin oligonucleotide H (FIG. 8B).

The selector-capture oligonucleotide has three requisite features. First, it must be of sufficient length to anneal effectively to the PENTAmer library. It should also be composed of a unique sequence opposite the restriction site where adaptor A was attached in PENTAmer library construction. Third, it contains an affinity tag, shown in FIG. 9 as biotin, permitting selective capture of ligated molecules under conditions that denature oligonucleotides that are not covalently joined. FIG. 8B illustrates how streptavidin-magnetic beads can immobilize biotin-tagged molecules. Washing with NaOH will denature double-stranded DNA and remove all non-covalently attached molecules.

It should be noted that the ligation of the hairpin and the selector-capture oligonucleotides can occur simultaneously, and the process does not have to be performed in a stepwise manner. In this scenario, both the hairpin and selector-capture oligonucleotides are added to the PENTAmer library, annealed, incubated with DNA ligase, then affinity purified.

### EXAMPLE 2

### Multiplexed Specific Primary Pentamer Isolation by 5′ End Ligation-Capture

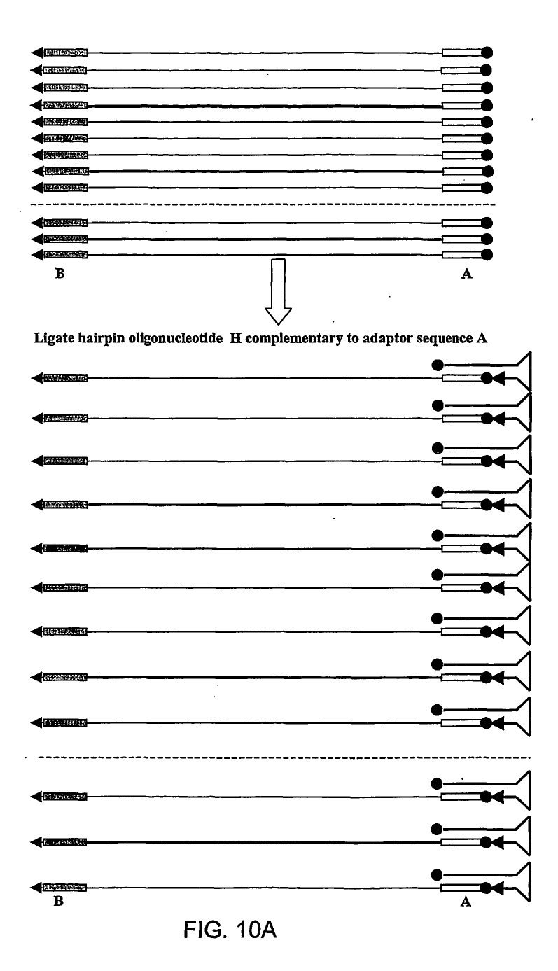

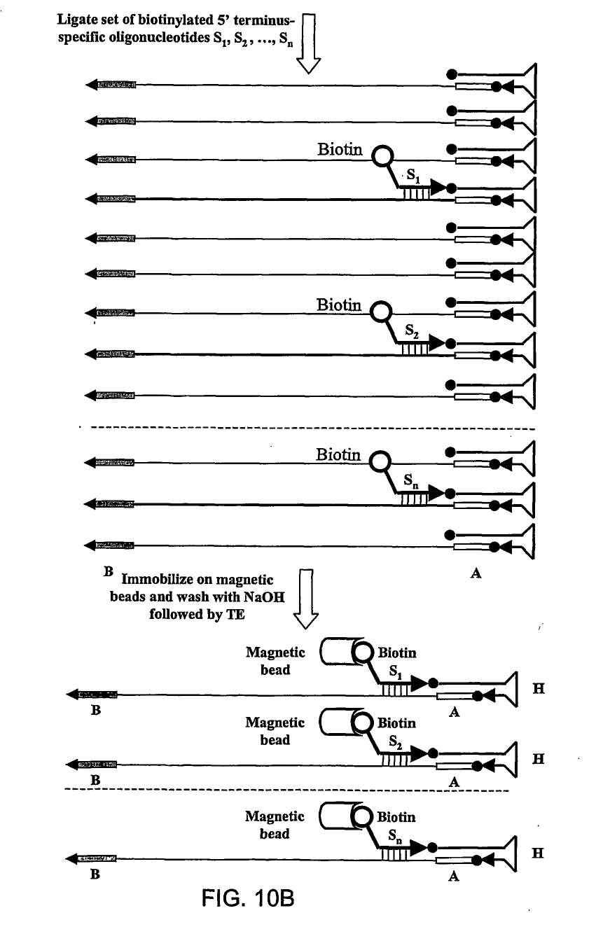

Multiple primary PENTAmers can be isolated by adaptation of the method described in Example 1. The first step, ligation of the hairpin oligonucleotide H to adaptor A, is the same. At this point, several different selector-capture oligonucleotides can be used to concomitantly isolate multiple PENTAmer species. The set of selector-capture oligonucleotides, each having a unique sequence, are designated S1 . . . Sn in FIGS. 10A and 10B. The PENTAmers of interest are then affinity captured. For example, as shown in FIGS. 10A and 10B, streptavidin-magnetic beads can be used to bind biotinylated selector-capture oligonucleotide ligation products. Washing with NaOH will remove all non-covalent (i.e., non-ligated) molecules. This example demonstrates that addition of several selector-capture oligonucleotides can permit isolation of multiple unique PENTAmer products from the same library.

Conversely, the same selector-capture oligonucleotide can be used to isolate similar PENTAmer molecules from different libraries. Different primary PENTAmer libraries, tagged with different versions of adaptor A, can be pooled. The combined libraries can then be selected with one or more selector-capture oligonucleotides to isolate the PENTAmers of interest. Captured products will all have the same complementary sequence to the selector-capture oligonucleotide(s), but can arise from different libraries. The source could be identified by using a library-specific version of adaptor A. It should be noted that variants of adaptor A require corresponding changes in the hairpin oligonucleotide H to maintain basepairing.

### EXAMPLE 3

### Reducing Pentamer Library Complexity by Ligation-Mediated Capture

Examples 1 and 2 outlined methods to isolate one or more specific PENTAmers from one or more libraries. This Example illustrates a method for systematically reducing the complexity of an entire PENTAmer library or combination of libraries. The separate pools can be placed in ordered arrays for analysis or further downstream processing.

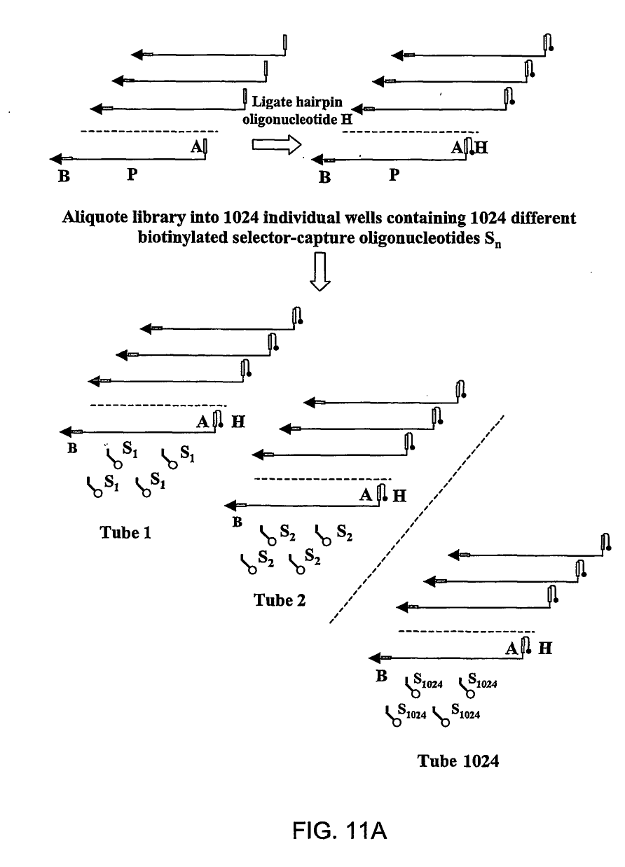

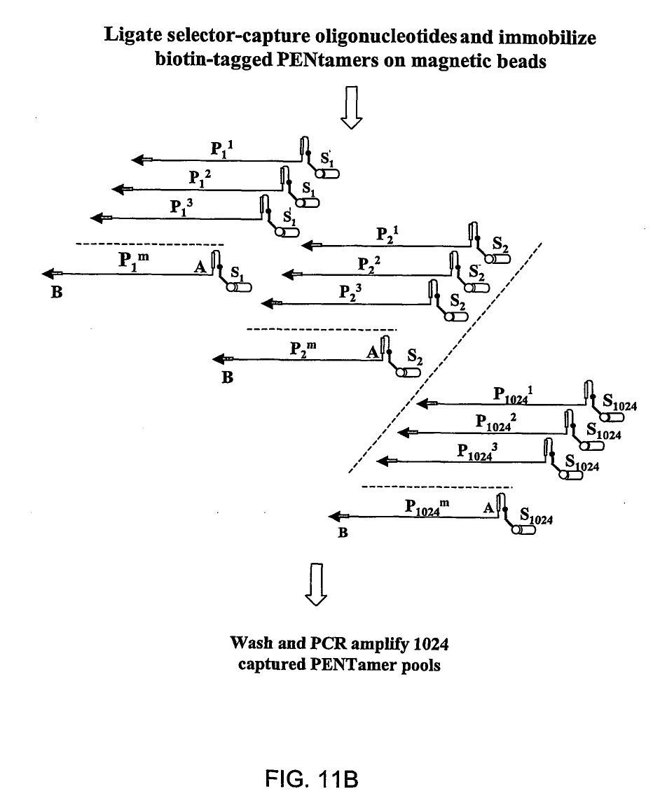

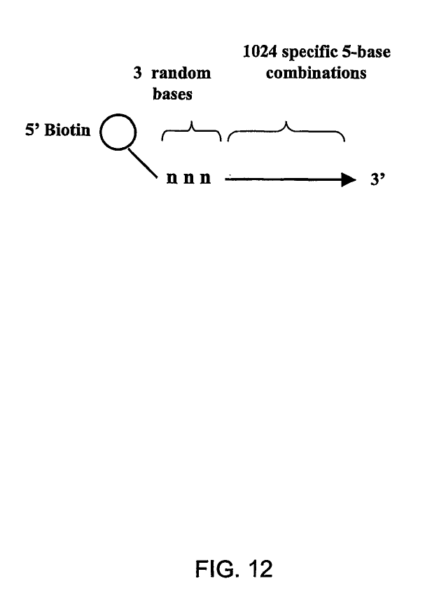

The hairpin oligonucleotide is ligated to the adaptor A as described in Example 1 (FIG. 11). Note that library-specific adaptor A and hairpin oligonucleotides can be used for simultaneous processing of multiple libraries. The library-specific adaptor A and hairpin oligonucleotides would allow identification of the isolated PENTAmer source, if desired. The library is then aliquoted to 1024 separate tubes or wells in a plate format. Each tube or well contains a unique specialized selector-capture oligonucleotide (FIG. 12). DNA ligase is added to each reaction, covalently attaching only PENTAmers complementary to the unique 5-base combination of the selector-capture oligonucleotide.

The 1024 specialized selector-capture oligonucleotides encompass all sequence possibilities complementary to the 5-bases of the PENTAmer adjacent to the hairpin oligonucleotide H and adaptor A duplex. These five defined bases are preceded by three randomized nucleotides at the 5′ terminus of the oligo (FIG. 12). The randomized bases ensure the presence of an oligonucleotide fraction that will have a total of eight contiguous bases of complementarity to the target PENTAmer molecules. An affinity tag is located at the 5′ terminus. Therefore, the defined 5-base combination will isolate PENTAmers complementary to the corresponding specific sequence, and the additional three randomized bases will ensure a fraction of the selector-capture oligonucleotides will have eight consecutive base pairs. Eight base pairs will permit efficient ligation of the selector-capture oligonucleotide to the appropriately paired PENTAmer target.

The products are purified by affinity capture, using streptavidin-magnetic beads to immobilize biotin-conjugated products, for example. Non-covalently attached molecules are removed by washing with NaOH to denature DNA duplex structures. Each pool can then be analyzed or amplified as desired.

B. Isolation of Complements to Specific Primary PENTAmers by Primer Extension-Capture and Subdivision of PENTAmer Libraries

Complementary molecules of individual PENTAmers can be isolated from a primary PENTAmer library using primer extension. One or more oligonucleotides are annealed to the primary PENTAmer library and extended using one of the commercially available DNA polymerases. The oligonucleotide contains an affinity tag for capture of the extended molecules. Examples 4 and 5 illustrate the method in capture of a single product and in capture of multiple products. Product molecules will contain the complementary DNA sequence to the primary PENTAmer targets.

Primer extension can also be used to subdivide the primary PENTAmer library. An oligonucleotide is annealed to the 3′ universal adaptor of the PENTAmer library. The terminal 3′ base(s) of this oligonucleotide can extend beyond the adaptor sequence, to provide selectivity for extension. DNA polymerase lacking 3′exo proofreading activity (for example, native Taq DNA polymerase) will not extend a 3′ mismatch, consequently only PENTAmers that base pair with the 3′ selective portion of the extension oligonucleotide will generate products. This method is described in Example 6.

### EXAMPLE 4

### Specific Primary Pentamer Isolation by Primer Extension-Capture

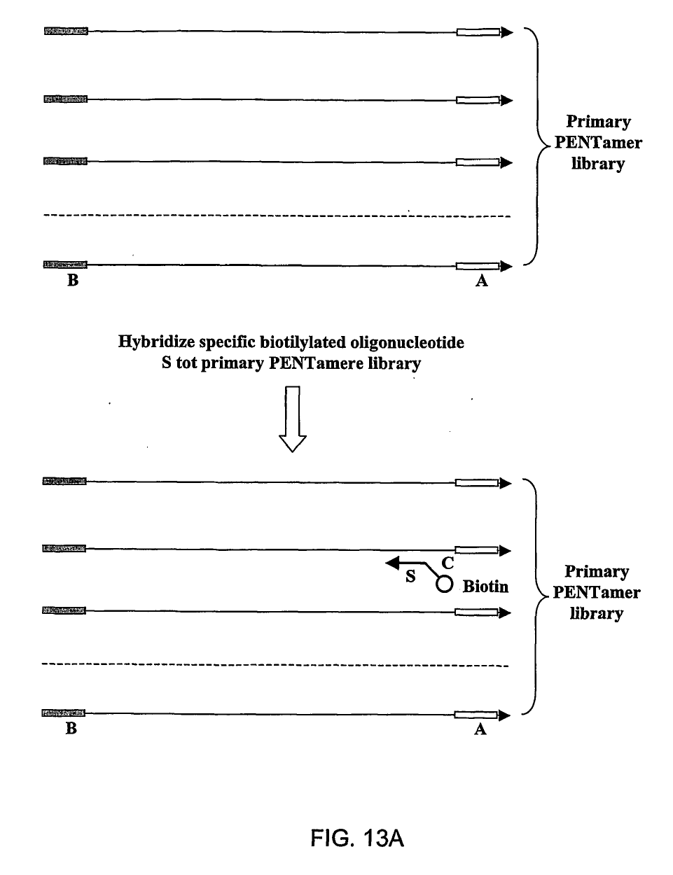

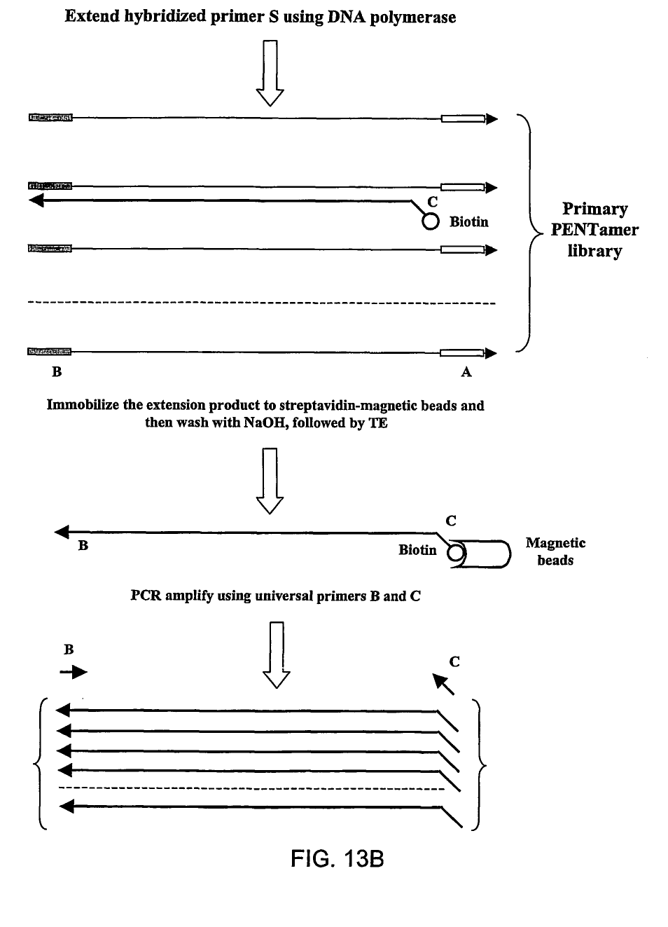

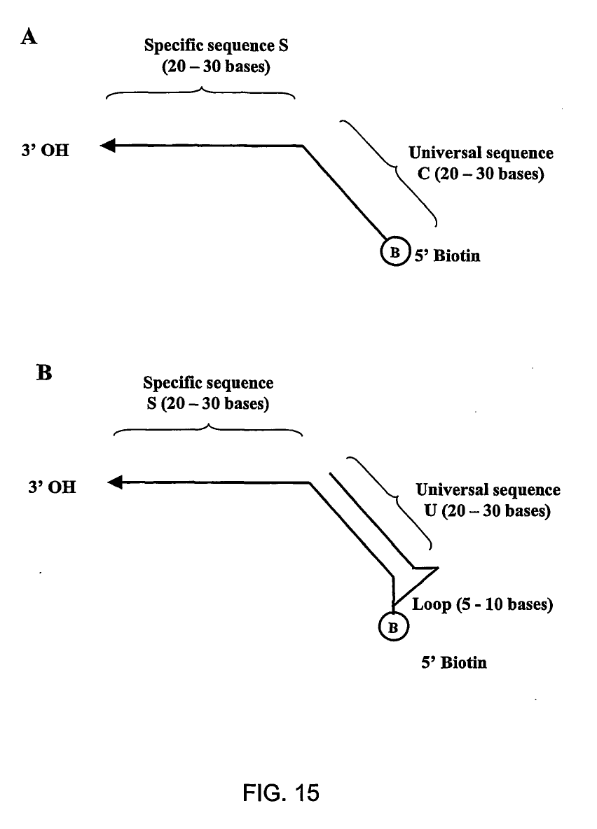

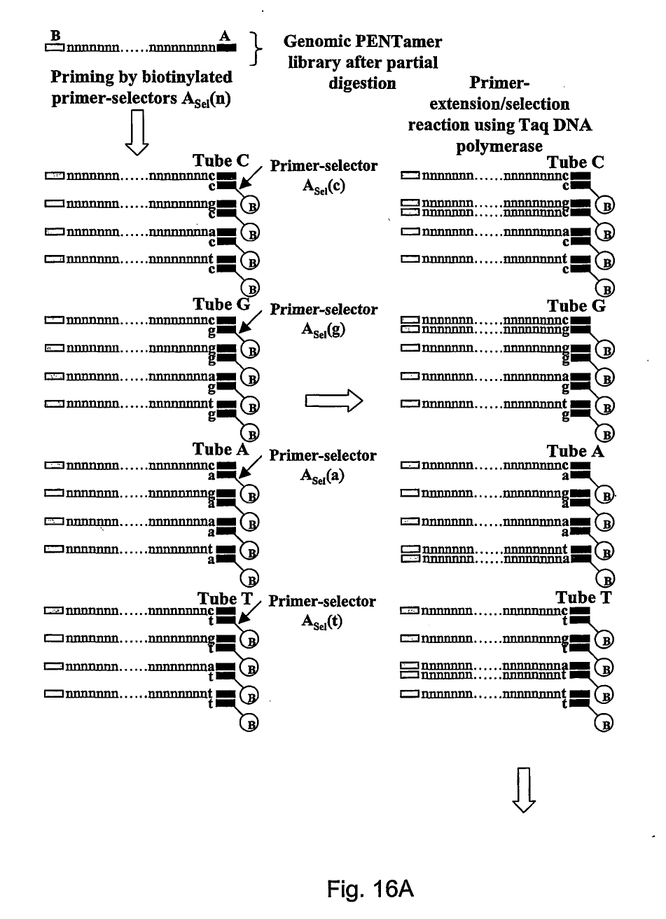

Complementary molecules to a specific primary PENTAmer can be generated by primer extension of an oligonucleotide that hybridizes to a unique DNA sequence within the primary PENTAmer (FIGS. 13A and 13B). The oligonucleotide is designed to have two parts, the 3′ region contains the sequence directed to the PENTAmer of interest (labeled S in FIG. 15), and the 5′ region contains a stretch of nucleotides whose sequence is not found in the PENTAmer (labeled U in FIG. 15). In addition, the oligonucleotide contains an affinity tag, such as biotin, for capture of products. To prevent non-specific hybridization of the oligonucleotide to the library, the 5′ region can have a hairpin structure shown on FIG. 15B. After annealing, the oligonucleotide is extended using DNA polymerase, which will synthesize a new complementary DNA strand to the PENTAmer of interest. Extension products are affinity captured and the DNA is denatured using NaOH. This permits removal of the annealed primary PENTAmer, leaving a single-stranded complementary DNA molecule (FIG. 13B).

The products can be amplified using PCR with oligonucleotides that anneal to regions B and U (FIGS. 13A and 13B). Region B is from the 5′ adaptor of the primary PENTAmer library. Region U is the 5 portion of the oligonucleotide used in the primer extension reaction. It should be noted that in this simple case, the primer extension oligonucleotide could be composed solely of region S. This same oligonucleotide would then be used in conjunction with oligonucleotide B for PCR amplification. The benefits of a two-part primer extension oligonucleotide are realized in the multiplexed format, described below, or in the future combination of multiple individually isolated products. For example, a combined pool of different products could be simultaneously amplified using oligonucleotides B and U, since they are universal to all products.

### EXAMPLE 5

### Multiplexed Specific Primary Pentamer Isolation by Primer Extension-Capture

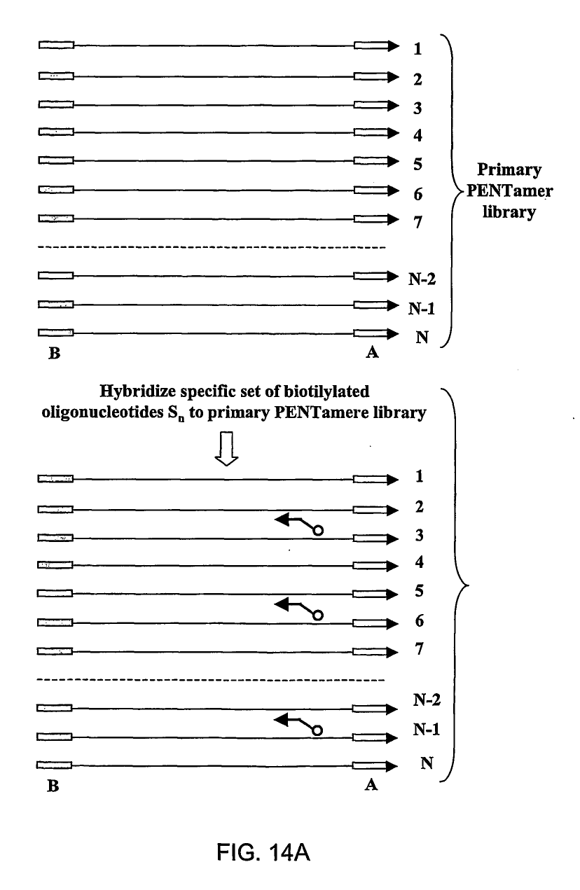

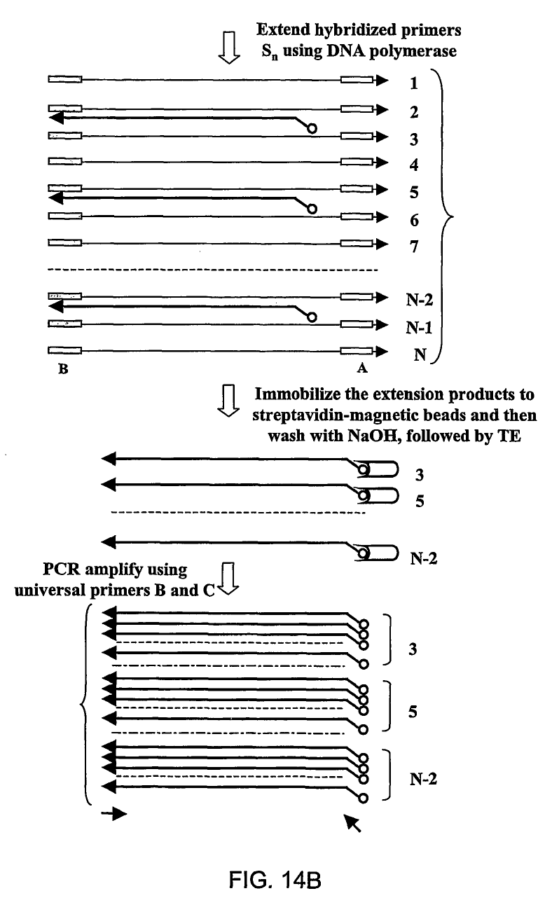

The method for generating primer extension products of multiple PENTAmers is the same as described in Example 4, except more than one oligonucleotide is used. The specific portion of the oligonucleotide, region S in FIG. 14A, will be unique for each primary PENTAmer of interest. However, region U of each oligonucleotide will be the same. Using several different oligonucleotides allows priming of their respective primary PENTAmers in the same reaction. Annealing, extension, and affinity capture are the same as in the single oligonucleotide example.

The primer extension products all contain the constant region U at the 5′ terminus. The two oligonucleotides, B and U, permit amplification of the molecules of interest by PCR (FIG. 14B). Oligonucleotide B anneals to the 5′ adaptor sequence of the primary PENTAmer and oligonucleotide U is composed of the 5′ half of the primer extension oligonucleotide.

Conversely, the same primer oligonucleotide can be used to isolate similar PENTAmer molecules from different libraries. Different primary PENTAmer libraries, tagged with different versions of adaptor ALS, can be pooled. The combined libraries can then be selected with one or more primer oligonucleotides to isolate the PENTAmers of interest. Captured products will all have the same complementary sequence to the S region of primer oligonucleotide(s), but can arise from different libraries. The source could be identified by using a library-specific region AN of the adaptor ALS.

### EXAMPLE 6

Reducing Pentamer Library Complexity by Primer Extension-Capture

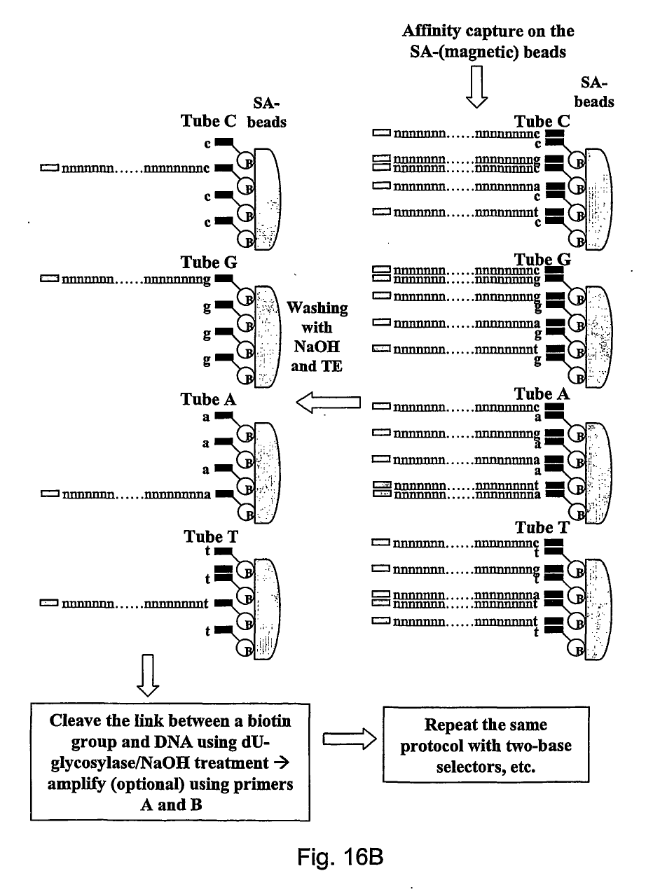

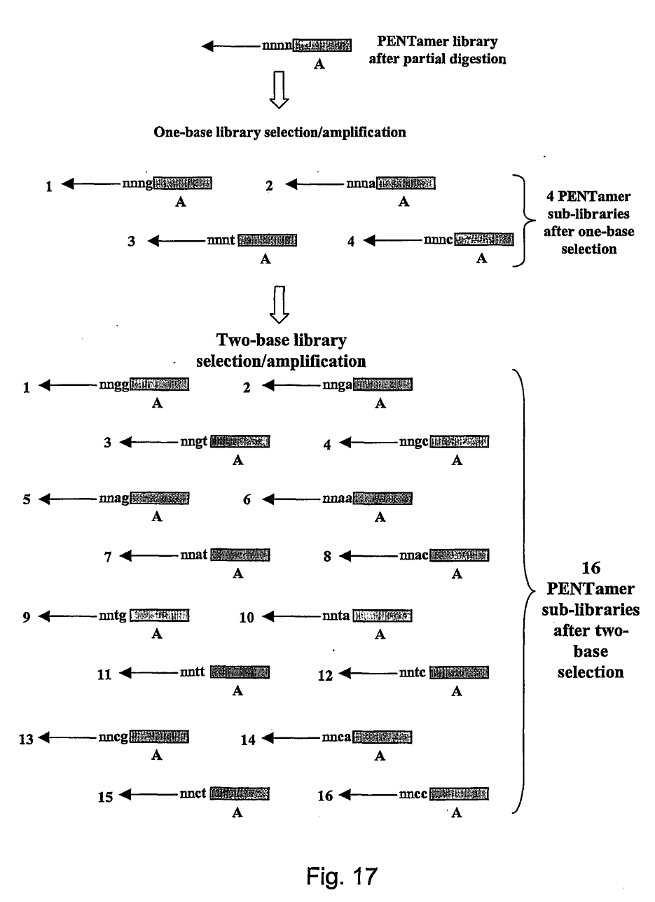

A primary PENTAmer library can be subdivided according to sequence adjacent to the 3′ adaptor A. A primer extension oligonucleotide complementary to adaptor A, but containing specific bases at the 3′ end beyond the adaptor sequence, will only be extended when the 3′ terminal bases are paired with the PENTAmer. The primer extension oligonucleotide is depicted as the ‘primer-selector’ in FIGS. 16A and 16B. Using an array of such oligonucleotides, primer extension products can be generated corresponding to the specific pairing of the terminal base(s). For example, oligonucleotides complementary to adaptor A but containing an additional 3′ A, C, G, or T will subdivide the PENTAmer library into the four corresponding pools (FIGS. 16A, 16B, and 17). Two additional bases would permit division into sixteen pools, and so on.

The product arrays could be set in a plate or chip format, separating each pool of products. Note that all products could be amplified by PCR using oligonucleotide A, without any additional 3′ bases, and oligonucleotide B.

C. Isolation of Specific PENTAmers and Subdivision of PENTAmer Libraries by PCR

This section describes the isolation of specific PENTAmers from a primary PENTAmer library and subdivision of a primary PENTAmer library into specific pools using direct PCR.

One or more sequence specific oligonucleotide primers are used to isolate specific PENTAmer molecules by conventional PCR. Examples 7 and 8 illustrate the method of isolation of single and multiple products, respectively. Product molecules will contain the complementary DNA sequence to the primary PENTAmer targets.

PCR can also be used to subdivide the primary PENTAmer library. One of the PCR primers is annealed to the 3′ universal adaptor of the PENTAmer library. The terminal 3′ base(s) of this selective primer can extend beyond the adaptor sequence to provide selectivity for extension. DNA polymerase lacking 3′exo proofreading activity (for example, native Taq DNA polymerase) will not extend a 3′ mismatch, consequently only PENTAmers that base pair with the 3′ selective portion of the primer will generate products. This method is described in Example 9.

### EXAMPLE 7

### Specific Primary Pentamer Isolation by PCR

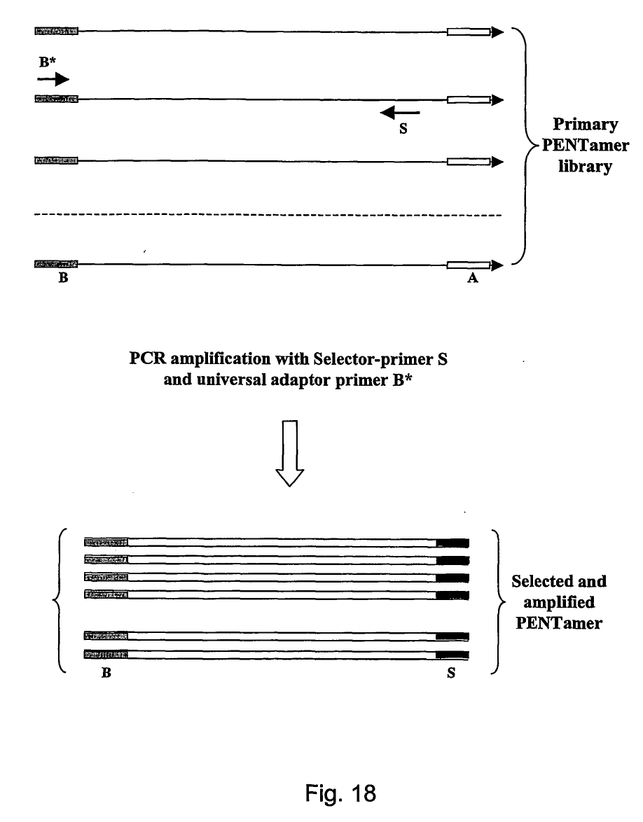

The isolation is performed in a single amplification PCR step (FIG. 18). The primer B* is complementary to adaptor B of the PENTAmer library. A sequence specific selector-primer S is complementary to known DNA sequence somewhere close to the adaptor A. If necessary, a second PCR reaction can be performed using nested primers B** and S′. The primer B** is complementary to an internal region of the adaptor B. A sequence specific selector primer S′ is complementary to known DNA sequence located closer to the adaptor B than the first priming site (S).

FIG. 18 illustrates how a PCR reaction can isolate a specific PENTAmer molecule using primer B* complementary to adaptor B of the PENTAmer library. Similar, the isolation procedure can be performed using primer A* complementary to the adaptor A of the PENTAmer library. In this case, a sequence specific selector-primer S should be complementary to known DNA sequence somewhere close to the adaptor B.

### EXAMPLE 8

### Multiplexed Specific Primary Pentamer Isolation by PCR

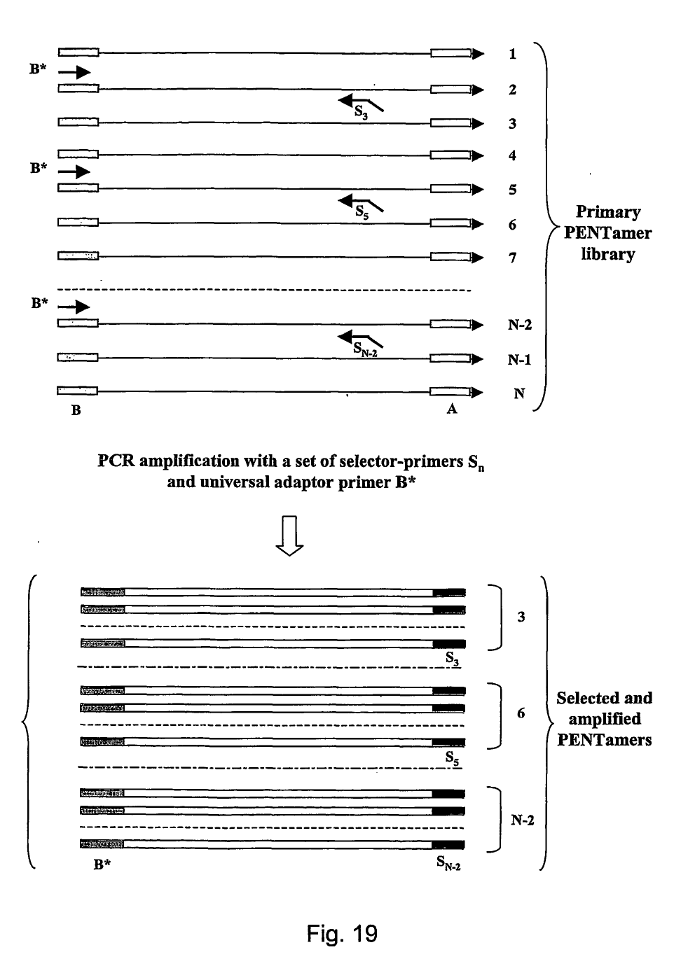

Multiple primary PENTAmers can be isolated by adaptation of the method described in Example 7. The isolation is performed in a single amplification PCR step FIG. 19. The primer B* is complementary to adaptor B of the PENTAmer library. Several different sequence specific selector primers Sn are used to isolate multiple PENTAmer species. The set of selector-primers, each having a unique sequence, are designated S₃, S₅ . . . S_{N-2} in FIG. 19. If necessary, a second nested multiplexed PCR reaction can be performed to increase specificity of the amplified products. Similar to the Example 7, the nested primer B** and the set of nested selector-primers S′₃, S′₅ . . . S′_{N-2} should be used. This example demonstrates that addition of several selector-primers can permit isolation of multiple unique PENTAmer products from the same library.

Conversely, the same selector-primer can be used to isolate similar PENTAmer molecules from different libraries. Different primary PENTAmer libraries, tagged with different versions of adaptor ALS, can be pooled. The combined libraries can then be selectively amplified with one or more selector-primer to isolate the PENTAmers of interest. Amplified products will all have the same complementary sequence to the selector-primer(s), but can arise from different libraries. The source could be identified by using a library-specific version of adaptor ALS.

### EXAMPLE 9

### Reducing Pentamer Library Complexity by Selective PCR

The two previous examples outlined PCR methods to isolate one or more specific PENTAmers from one or more libraries. This example illustrates a selective PCR method for systematically reducing the complexity of an entire PENTAmer library or combination of libraries. The separate pools can be placed in ordered arrays for analysis or further downstream processing.

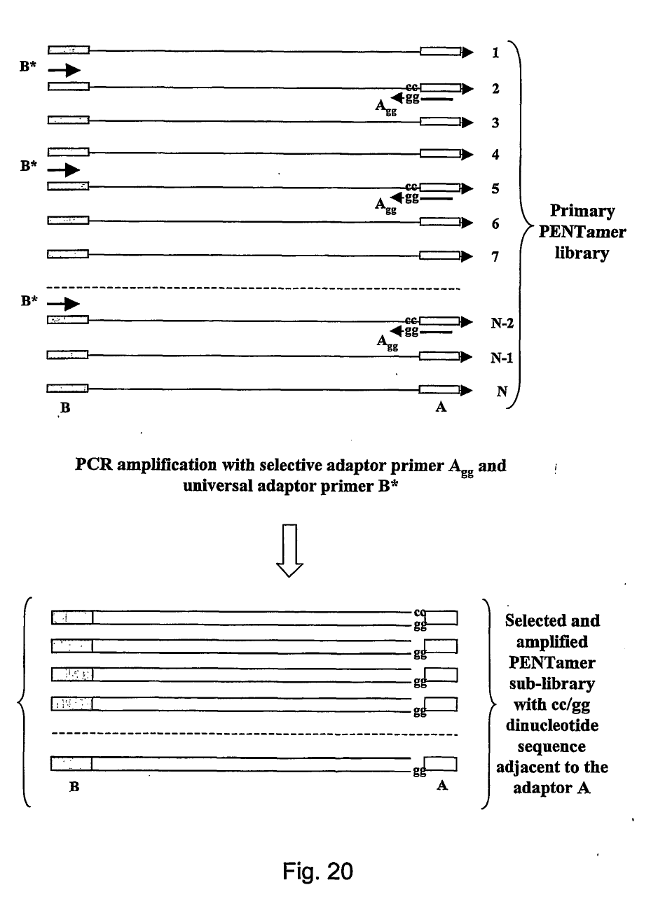

The isolation is performed in a single amplification PCR step (FIG. 20). The library is aliquoted to multiple separate tubes or wells in a plate format. Each tube or well contains a specialized primer selector and primer B*. The primer B* is complementary to adaptor B of the PENTAmer library. All but a few bases at the 3′ end of the primer selector are complementary to the adaptor sequence A. FIG. 20 illustrates the case when primer selector Agg has two selective bases (GG) at the 3′ end, but the number of selective bases can be three or more. The 3′ bases of the primer selector are hybridized to the DNA region immediately adjacent the adaptor sequence A and enable the amplification of PENTAmer molecules with selected composition next to the adaptor A sequence. Two-base selection would result in 16 different PENTAmer sub-libraries of reduced complexity. The example presented in FIG. 20 shows the selection of PENTAmers with CC/GG base composition in the region adjacent to the adaptor A. Use of three-base selection can increase the number of sub-libraries to 64, although the method might be limited by the lower specificity of three-base selection.

XIX. Using Unordered Recombinant PENTAmer Libraries for SNP Detection

Genomic libraries of recombinant Type I or Type II PENTAmers (as described in U.S. patent application Ser. No. 09/860,738) can be used to amplify large regions of a genome. These processes of amplification can be designed to identify SNPs from very large regions of human, animal and plant genomes. SNP analysis using recombinant PENTAmer libraries is more efficient than PCR, because a) the size of the region amplified can be up to 100 times larger than the size of regions that can be amplified by conventional PCR; b) only a single set of amplification primers are necessary to amplify the large region, compared to PCR that would require up to 100 sets of primers to amplify the same region; c) PENTAmer amplicons are of small, controllable size and therefore ideal for discrimination of SNPs by hybridization; and d) because recombinant PENTAmers are made using an intramolecular recombination reaction, the amplification process can be designed to determine haplotypes as well as genotypes.

The process of amplifying a region of DNA using PENTAmer molecules is called “positional amplification.” Because positional amplification can amplify a very large region adjacent to a kernel sequence, it can be used as a general tool to produce DNA molecules for analysis. Specific aspects of positional amplification make it extremely useful for haplotyping and genotyping individual humans, animals, and plants.

U.S. Pat. No. 6,197,557, incorporated by reference herein, describes how amplifiable DNA molecules complementary to the ends of DNA fragments are produced by attachment of specialized adaptor molecules to the ends of the fragments, performing a controlled nick-translation reaction using each terminus of the fragments to synthesize DNA strands of controlled length that are complementary to the termini of the fragments, and amplifying those fragments using conventional technology. U.S. patent application Ser. No. 09/860,738 describes how genomic libraries of amplifiable nick-translation products can be produced and used to amplify large regions of the genome for sequencing and other analytical purposes. The present invention describes various methods by which the amplified nick-translation products (PENTAmers) can be used to detect single-nucleotide polymorphisms in the DNA of an individual.

As described in U.S. patent application Ser. No. 09/860,738, recombinant PENTAmer libraries are made in the following way. Genomic DNA fragments of heterogeneous length are created by partial restriction digestion or other means, followed by attachment of specialized adaptor molecules comprising nicks to the ends of the fragments, performing a nick translation reaction to create DNA strands with 5′ ends complementary to the termini of the fragments and 3′ ends complementary to regions a controlled distance from the ends of the fragments, and attaching adaptor sequences to the 3′ ends of the nick-translate molecules. An intramolecular recombination reaction is performed to attach the two ends of each of the fragments, bringing the nick-translation products complementary to DNA sequences at the proximal and distal ends of the fragments adjacent to each other in either a linear or circular molecules. The recombinant PENTAmers are amplified by primer extension, PCR, rolling circle amplification, or other method.

FIG. 21 schematically illustrates how an intramolecular recombination event between primary PENTAmers at the two ends of a DNA fragment can be used to form a circular recombinant PENTAmer that can be amplified using inverse PCR. If the primers are complementary to known sequences located near the proximal end of the fragment, then PCR can amplify the sequences adjacent to the distal end of the fragment, even if the sequences at the distal end are unknown. U.S. patent application Ser. No. 09/860,738 describes methods to synthesize primary PENTAmers, methods to perform intramolecular recombination, and methods to amplify the recombinant PENTAmers in locus-independent and locus-specific manners.

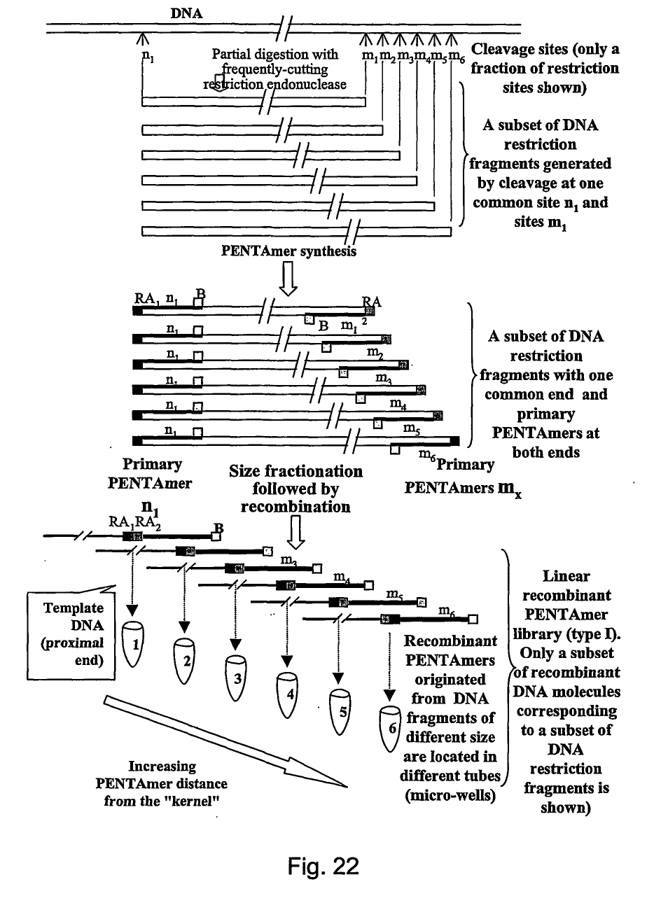

FIG. 22 illustrates how partial digestion with a restriction enzyme can be used to create nascent PENTAmers that can be size-fractionated to separate linear recombinant PENTAmers that have common ends at a proximal restriction site, n1 and opposite ends at different restriction sites, m1, m2, m3, . . . , located increasing distances from the proximal restriction site n1. The PENTAmers illustrated are those that have a common proximal end, however in a genomic preparation PENTAmers with proximal ends terminating at every restriction site would be represented.

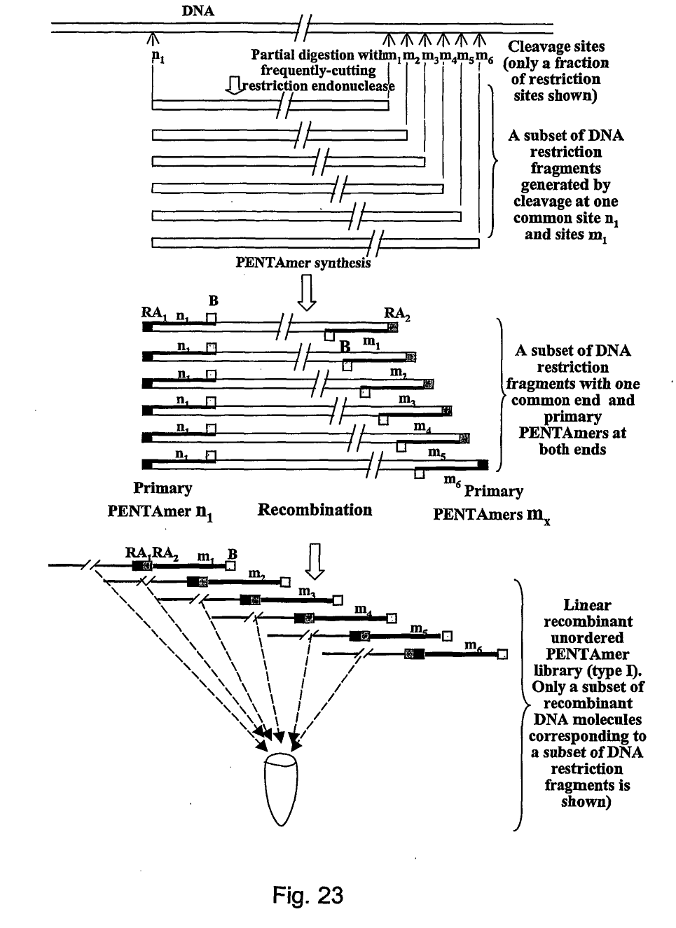

FIG. 23 illustrates how omission of the size separation step shown in FIG. 21 leads to a pool of recombinant PENTAmers that comprise an unordered library of amplifiable PENTAmer that terminate at a family of restriction sites. The PENTAmers illustrated are those that have a common proximal end, however in a genomic preparation PENTAmers with proximal ends terminating at every restriction site would be represented.

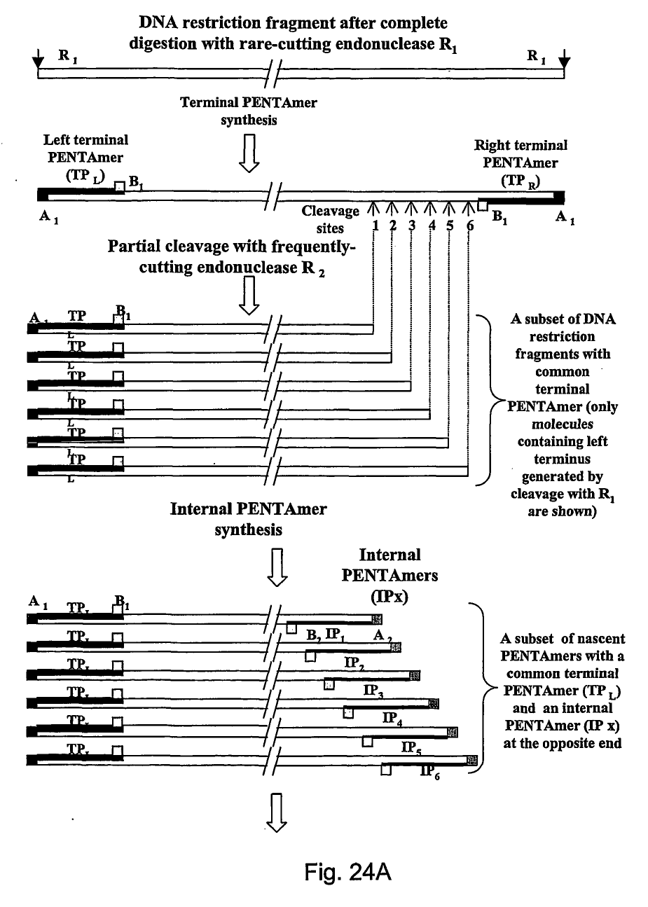

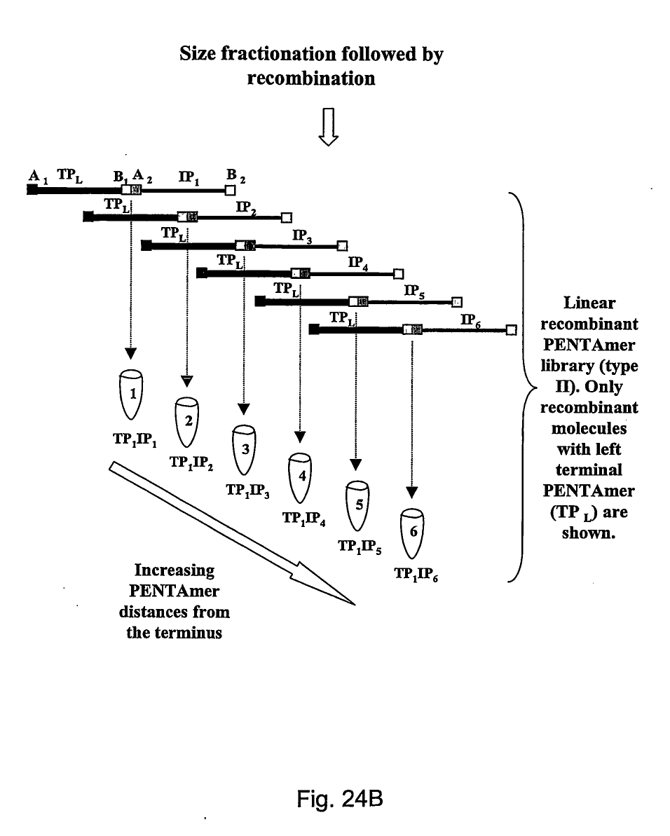

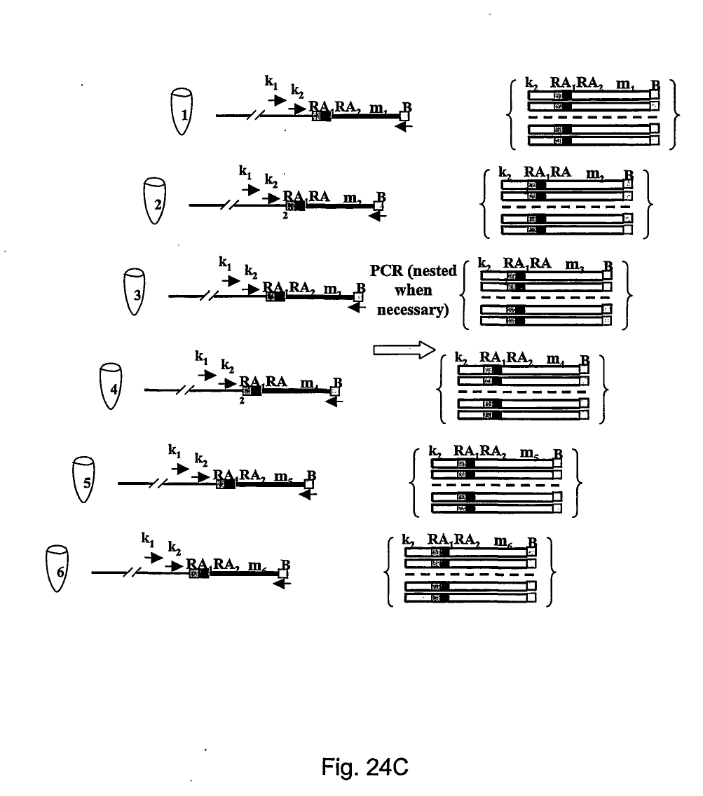

FIGS. 24A, 24B, and 24C show how an initial complete restriction digestion with an infrequently-cutting restriction endonuclease and a partial digestion with a second restriction enzyme can also be used to create an ordered recombinant PENTAmer library. Omission of the size separation step would also produce an unordered PENTAmer library, as in FIG. 23. FIG. 24C shows how amplification of the linear recombinant PENTAmers from each size fraction using PCR primers (nested primers are shown) complementary to a sequence (the kernel) near the proximal ends of the fragments can be used to achieve locus-specific amplification of an ordered set of distal sequences.

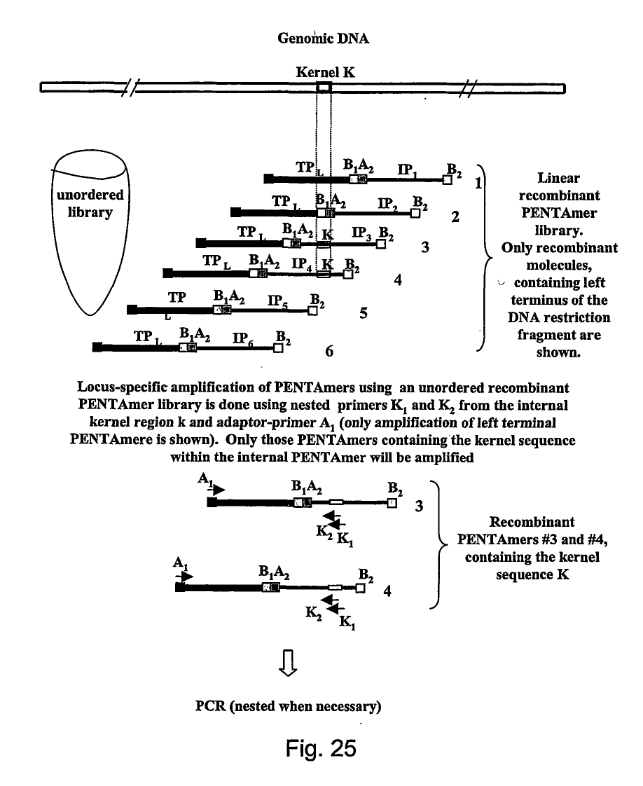

FIG. 25 illustrates the principle of locus-specific amplification of the recombinant. PENTAmers in an unordered library that contain kernel sequences. The example shows how only the PENTAmers containing the kernel sequence are amplified.

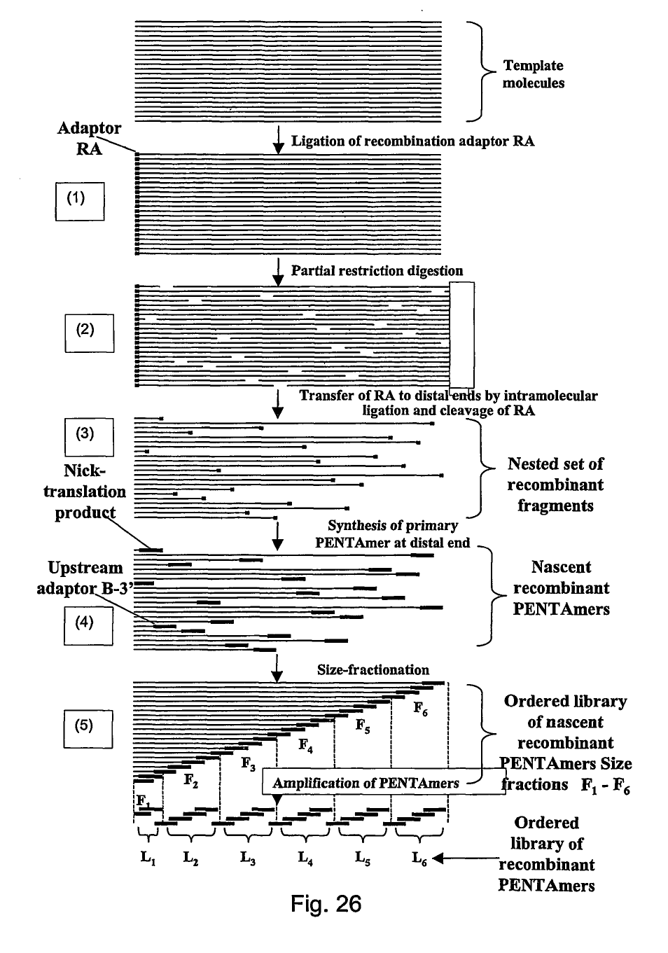

FIG. 26 illustrates how the ordered PENTAmers in a library represent sequences different distances from a proximal end.

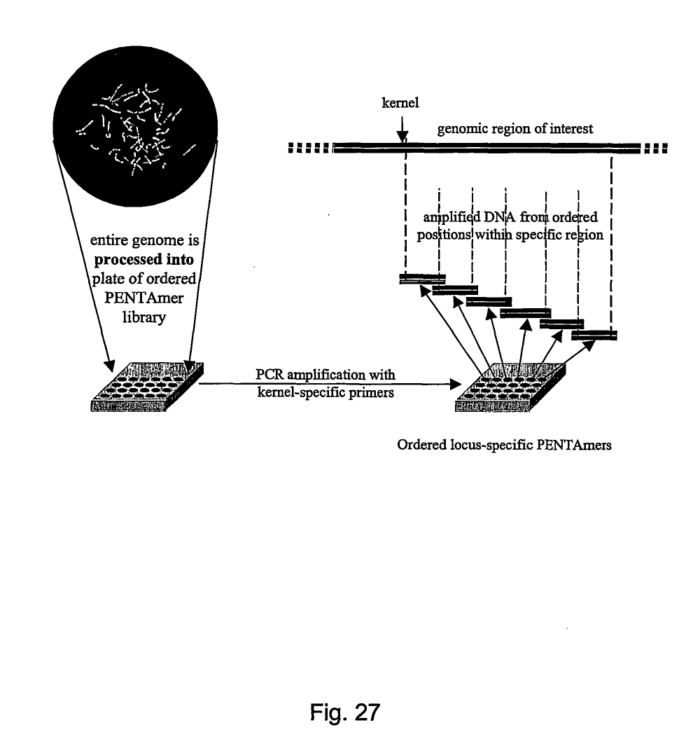

FIG. 27 illustrates how an entire genome is first processed into an ordered PENTAmer library contained within the wells of a microwell plate, and amplified with the same kernel primers in each well to produce amplicons that cover different positions within a large genomic region of interest that is to one side of the kernel.

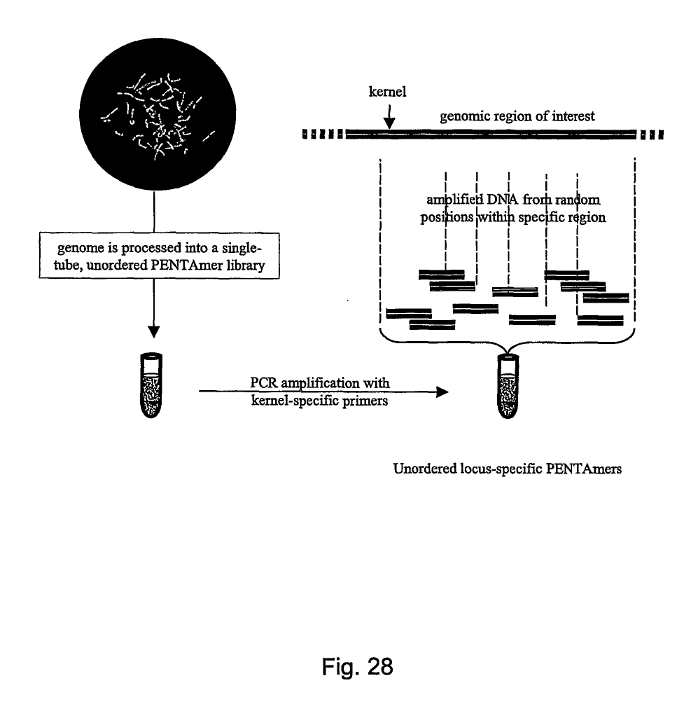

FIG. 28 illustrates how a genome is first processed into an unordered PENTAmer library that is contained within a single tube, and amplified with kernel primers to produce a mixture of amplicons of uniform length that cover a large region of interest. Because the nascent PENTAmers have not been separated by size the size of the region complementary to the amplicons is only limited by the maximum size of intact DNA fragments that are present in the solution. The only sequence that must be known for the amplification is the sequence chosen to be the kernel. If the kernel primers are complementary to more than one site in the genome, more than one region will be amplified.

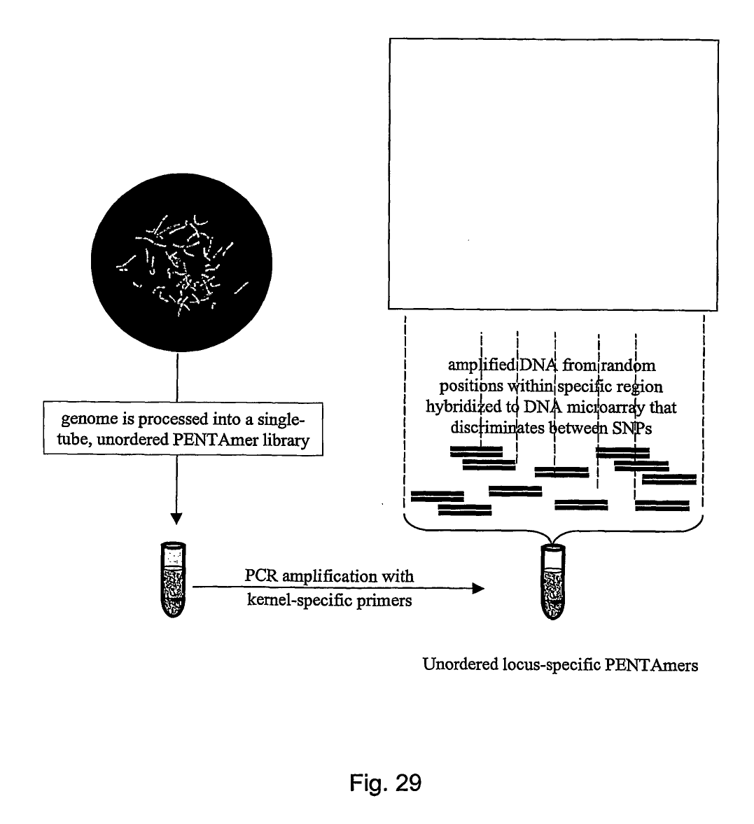

FIG. 29 illustrates how the amplified unordered PENTAmer library can be hybridized to a DNA microarray that is designed to test whether a specific base is present at a specific location within the sequence. The microarray does not have to “test” the sequence at all positions, but only a subset of those in the genome or in the amplified fraction of the genome; e.g. the amplification might be designed to amplify m loci in the genome, whereas the microarray might only test for the presence of n SNP, where m>n.

The amplification of unordered PENTAmer libraries can be multiplexed by simple multiplexing of the PCR reactions. For example, if ten sets of kernel primers are used in the same amplification reaction, ten loci can be simultaneously amplified. Each locus can be hundreds of thousands of bases long, if desired. Up to 20 sets of primers can be used to perform conventional PCR in a multiplexed mode. Thus, it is feasible to use 20 sets of kernel primers to simultaneously amplify up to 20 distinct large regions in a genome. For purposes of SNP analysis, the regions could contain specific genes or sets of genes responsible for drug metabolism, responsible for a multigenic disease such as asthma, or multiple genes linked to a common disease such as colon cancer. The amplicons from different loci can be differentially labeled by attaching a tag to the kernel primers. For example, different kernel primers can be labeled with different fluorescent dyes detectable in a fluorimeter, different mass labels detectable in a mass spectrometer, or by different sequences detectable by hybridization to a DNA microarray.

For purposes of detecting a large number of SNPs (e.g., thousands, tens of thousands, hundreds of thousands, or millions) from a single tissue sample, the original DNA sample must be amplified many times to provide sufficient material for analysis. This amplification must be done in such a way that many sites are amplified to the same extent, without loss of some sites. Recombinant PENTAmers can be amplified in a locus-independent fashion using primers complementary to the terminal adaptors. Locus-independent amplification of the entire genomic library (amplification en masse) is an important step in detection of genome polymorphisms, because it increases the number of copies of the molecules which increases the number of SNP assays that can be performed given a limited amount of DNA collected from an individual human, animal or plant.

Significant for detection of SNPs in a single, large, contiguous region of the genome is locus-specific amplification of the recombinant PENTAmers as ordered or unordered libraries of molecules using primers that are complementary to a single kernel sequence. The size of the contiguous region is limited by the maximum size of DNA fragment that can be produced without nicks or breaks, e.g., as large as 500,000 bases. Experimental data shown in U.S. patent application Ser. No. 09/860,738 shows how a 50 kb region of DNA in a viral genome can be amplified using recombinant PENTAmers.

Unordered PENTAmers are created when the nascent PENTAmers are not separated according to size before amplification. This results in a large region of the genome being amplified as molecules of uniform size in a single tube. If recombinant PENTAmer libraries are created in this way, their locus-specific amplification produces a pool of molecules covering a region as large as 500 kb. These molecules can be shotgun sequenced or used for non-sequencing applications. The inherent advantages over PCR in these applications are 1) only a single priming site rather than two priming sites is necessary; 2) the amplimers are of short, uniform length, which is ideal for labeling and hybridization; and 3) the amplimers cover larger regions.

After amplification, the locus-specific PENTAmers can be used to discover and validate new polymorphisms, e.g., SNPs, deletions, amplifications, etc., or detect known polymorphisms in the DNA from individual organisms such as human patients. Some of the tools currently used to detect polymorphisms using PCR amplification would be more powerful using amplified PENTAmers, because of the three factors mentioned.

Tiled oligonucleotide microarray hybridization (e.g., to an Affymetrix array) can be used to detect single base changes in a genome (Cantor and Smith, Genomics, John Wiley & Sons, Inc., N.Y., 1999). Fifteen to thirty oligonucleotide features are often employed to determine which specific base is present at a specific position in the sequence. Therefore, a microarray with 600,000 features could detect up to 20,000 specific SNPs in a sample. Unfortunately, amplification of DNA to detect that number of SNPs might require up to 20,000 PCR reactions, prohibitively expensive, as well as time and material limited. Far fewer amplification reactions would be required to amplify the same amount of DNA from a recombinant PENTAmer library.

Alternatively, sequencing by hybridization can be used to resequence every base of the amplified region. Different specific SNPs within the amplified region can be tested using single base extension, pyrosequencing, oligonucleotide ligation assay (OLA), rolling circle amplification, strand invasion, or other techniques (Cantor and Smith, Genomics, John Wiley & Sons, Inc., N.Y., 1999).

Recombinant PENTAmers are useful for studies of haplotypes, i.e., the polymorphisms that are present in cis, i.e., located on the same copy of the chromosome (because they were inherited from one parent), or in trans, i.e., located on the chromosomes inherited from different parents. This information is significant, because many functional characteristics of genes and sets of genes are determined by whether multiple polymorphisms occur on the same copy of the chromosome and therefore create affect multiple alterations to the same protein molecules. Sometimes different genetic alleles function in cis to complement each other by producing proteins that have substantially different properties than if the alleles are present on separate chromosomes and give rise to separate protein molecules. Haplotype-specific amplification of PENTAmer libraries can be achieved using kernel primers that are specific for one allele, e.g., having a 3′ end complementary to one allele but not another. PCR of genomic DNA is usually unable to amplify a region larger than 5-10 kb, which is not large enough to cover many human genes, and the amplicons are then too large to effectively analyze. Allele-specific amplification of a large region as PENTAmers can produce short amplicons covering distances sufficient large to completely represent the largest human genes and even sets of functionally related genes that are in close proximity in the genome.

SNP Detection Using Amplified PENTAmer Libraries

Single nucleotide polymorphisms (SNPs) can be screened from pools of selected and amplified PENTAmers. Methods to isolate specific PENTAmers are illustrated in the Examples herein. The following examples describe how one or more SNPs can be detected in the PENTAmer pool(s). Fluorescently labeled products are generated from direct primer extension reactions or by ligation of fluorescent oligonucleotides to primer extension products. Both the extension reaction and the ligation reaction are highly sensitive to nucleotide identity. This specificity is exploited in the SNP detection methods. Electrophoretic separation of products identifies the target SNP, allowing analysis of several SNPs at the same time.

The examples rely on capillary electrophoresis for resolution of products. However, any DNA separation technology that can discriminate fluorescent dye types and/or molecule size is applicable. The last example shows how DNA oligonucleotide arrays on a plate or chip can be used to screen for SNP detection products.

### EXAMPLE 10

### Detection of Multiple SNPS in One DNA Sample Using Primer Extension Assay and Size Separation

Selected and amplified PENTAmers can be screened for the presence of multiple SNPs between alleles within a sample (FIG. 30). Fluorescently tagged oligonucleotides are designed to anneal adjacent to a known SNP location. The 3′ base of the oligonucleotides is varied using each complement to the known SNP location. The identity of the 3′ base of the oligonucleotide is marked using a different fluorescent dye in the oligonucleotide. Therefore, depending on the SNP identity, only the oligonucleotide with a complementary 3′ end will pair and be competent for extension with DNA polymerase. Mismatched 3′ oligonucleotides will not be extended due to the sensitive nature of DNA polymerase.

The size of primer extension products for a particular SNP location will be unique for that SNP. Each SNP analyzed by this method will produce discrete extension products that are of uniform fluorescence or of mixed fluorescence. Uniform fluorescence indicates the same fluorescently tagged oligonucleotide was extended on both alleles, while mixed fluorescence indicates a different oligonucleotide was extended on each allele. Specific products can be resolved by capillary electrophoresis. The resolution of different sized products enables many SNPs to be analyzed in the same reaction.

### EXAMPLE 11

### Detection of Multiple SNPS in One DNA Sample Using Primer Extension/Selective Ligation Assay and Size Separation

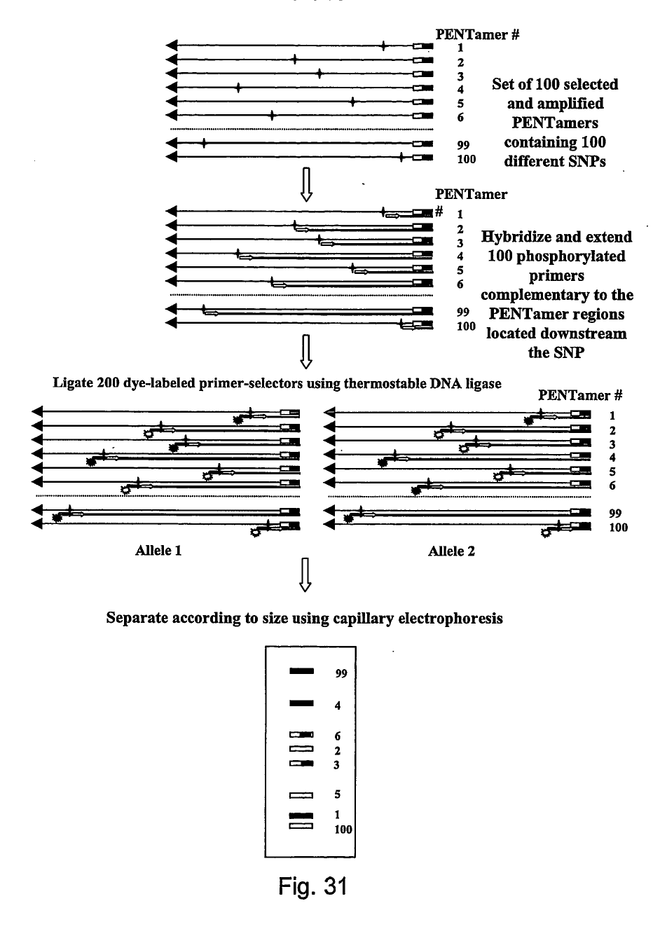

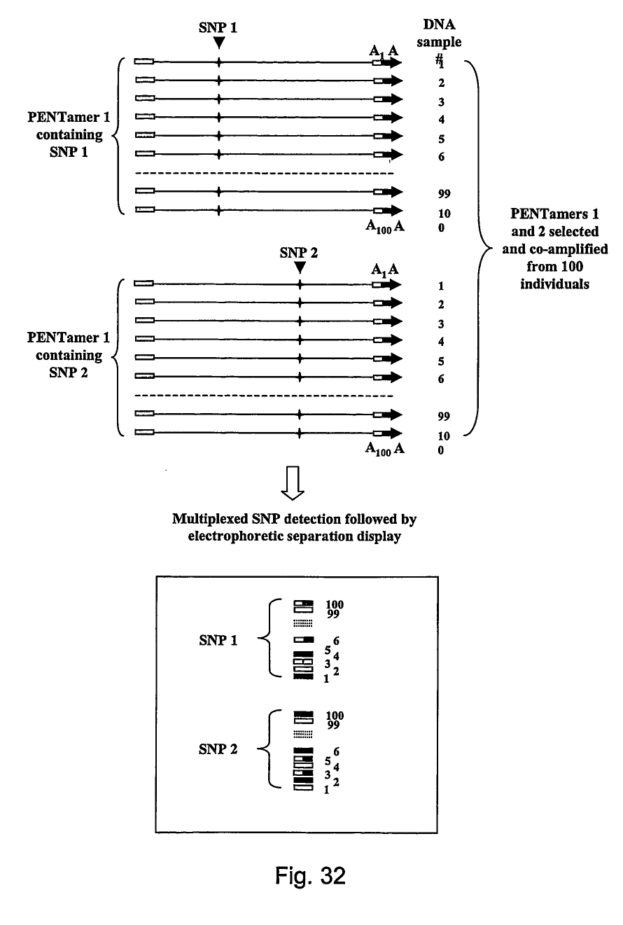

Base pairing identity at the site of DNA ligation can be used to discriminate SNPs (FIG. 31). This method is an adaptation of Example 10, except that ligation is used in place of extension as the selective event. An oligonucleotide is annealed with its 5′ end adjacent to a known SNP location. This oligonucleotide is extended by primer extension producing a product of discrete length from the SNP location. Next, fluorescently tagged oligonucleotides are annealed opposite the SNP from the first oligonucleotide. The 3′ terminal base of the fluorescently tagged oligonucleotide is varied to accommodate all pairing combinations with the known SNP. Each oligonucleotide variant is tagged with a unique fluorescent dye. The mixture is then incubated with DNA ligase, which will covalently join primer extension products with only fluorescently tagged oligonucleotides whose 3′ base is complementary to the SNP. Products are then resolved by size, with uniform fluorescence indicating the same nucleotide at each allele and mixed fluorescence indicating different bases between alleles at the SNP location.

### EXAMPLE 12

### Multiplexed Analysis of Several SNPS in Multiple DNA Samples Using Size Separation Display

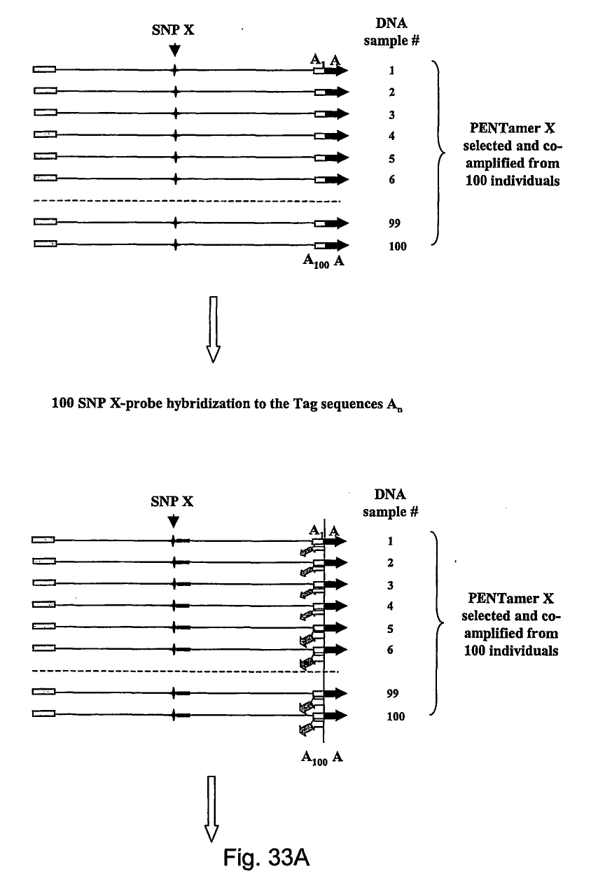

PENTAmers from multiple individuals can be screened for SNPs using either of the methods described in Examples 10 and 11. For this application, the PENTAmers must contain a uniquely sized portion of the A adaptor (FIG. 32). The PENTAmer source can thus be identified by the difference in size of primer extension products. Products generated by either Example 10 or 11 are resolved by electrophoresis resulting in clusters of products for each SNP analyzed. For example, the product of SNP 1 analysis will be longer than the product of SNP 2 analysis (FIG. 32). Within the pool of SNP 1 products there are different sized products corresponding to changes in the A adaptor. The A adaptor can contain 1 to 100 extra bases or units of bases unique to each source, as shown in FIG. 32 This method will permit analysis of as many SNPs and unique sources as long as products from each SNP will not overlap with size variations in the A adaptors (i.e., the SNPs must be far enough apart to prevent the clusters of products from A adaptor variation from being the same size). The location of SNPs analyzed and the number of DNA samples can be adjusted to ensure effective resolution of products.

### EXAMPLE 13

### Detection of One SNP in Multiple DNA Samples by One Base Primer Extension-Labeling Reaction and Hybridization to Oligo-Chip

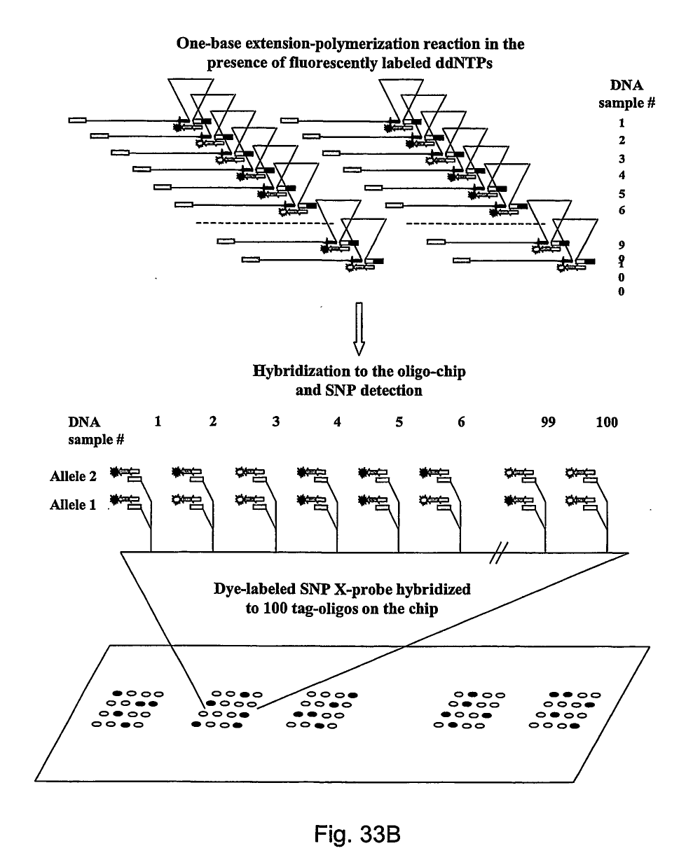

A single SNP can be detected in DNA samples from multiple individuals. PENTAmers from each individual must contain a unique sequence tag with the A adaptor region. This tag is designated A₁ to A₁₀₀ in FIG. 33A. A two-part oligonucleotide is used to discriminate the SNP identity for each unique A adaptor (FIGS. 33A and 33B). The 5′ region of the two-part oligonucleotide is complementary to the unique sequence tag within the A adaptor of each source. Therefore, there is a unique two-part oligonucleotide required for each DNA source. The second part of the two-part oligonucleotide, consisting of the 3′ region, is complementary to the region located immediately 5′ of the SNP of interest.

The two-part oligonucleotide is first annealed to the unique region of the A adaptor. The 3′ region of the two-part oligonucleotide can then anneal to the region immediately 5′ of the SNP of interest. Flexibility of the single-stranded PENTAmer will permit the length of DNA between the A adaptor and the SNP location to loop out, bringing the A adaptor and SNP region close together. Once both halves of the two-part oligonucleotide are annealed, the mixture is incubated with all four dideoxynucleotide triphosphates, each with a unique fluorescent tag, and DNA polymerase. The polymerase will incorporate the fluorescently tagged dideoxynucleotide corresponding to the base complement of the SNP of interest. Products can then be hybridized to an array of oligonucleotides, each position having one of the unique adaptor A sequences. SNPs from each source can be read by fluorescence at the corresponding position on the plate or chip array.

All of the methods and compositions disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

### Patents

U.S. Pat. No. 4,563,419

U.S. Pat. No. 4,656,127

U.S. Pat. No. 4,683,195

U.S. Pat. No. 4,683,202

U.S. Pat. No. 4,751,177

U.S. Pat. No. 4,800,159

U.S. Pat. No. 4,883,750

U.S. Pat. No. 5,075,216

U.S. Pat. No. 5,143,854

U.S. Pat. No. 5,202,231

U.S. Pat. No. 5,219,726

U.S. Pat. No. 5,279,721

U.S. Pat. No. 5,296,375

U.S. Pat. No. 5,302,509

U.S. Pat. No. 5,304,487

U.S. Pat. No. 5,424,186

U.S. Pat. No. 5,547,861

U.S. Pat. No. 5,578,832

U.S. Pat. No. 5,599,668

U.S. Pat. No. 5,610,287

U.S. Pat. No. 5,633,134

U.S. Pat. No. 5,719,028

U.S. Pat. No. 5,837,832

U.S. Pat. No. 5,837,860

U.S. Pat. No. 5,840,873

U.S. Pat. No. 5,843,640

U.S. Pat. No. 5,843,651

U.S. Pat. No. 5,846,708

U.S. Pat. No. 5,846,717

U.S. Pat. No. 5,846,726

U.S. Pat. No. 5,846,729

U.S. Pat. No. 5,849,487

U.S. Pat. No. 5,853,990

U.S. Pat. No. 5,853,992

U.S. Pat. No. 5,853,993

U.S. Pat. No. 5,856,092

U.S. Pat. No. 5,856,174

U.S. Pat. No. 5,858,659

U.S. Pat. No. 5,861,242

U.S. Pat. No. 5,861,244

U.S. Pat. No. 5,863,732

U.S. Pat. No. 5,863,753

U.S. Pat. No. 5,866,331

U.S. Pat. No. 5,882,864

U.S. Pat. No. 5,888,819

U.S. Pat. No. 5,904,824

U.S. Pat. No. 5,905,024

U.S. Pat. No. 5,910,407

U.S. Pat. No. 5,912,124

U.S. Pat. No. 5,912,145

U.S. Pat. No. 5,919,630

U.S. Pat. No. 5,925,517

U.S. Pat. No. 5,928,862

U.S. Pat. No. 5,928,869

U.S. Pat. No. 5,932,413

U.S. Pat. No. 5,935,791

U.S. Pat. No. 6,045,996

WO 86/03782

WO 88/10315

WO 89/06700

WO 89/01025

WO 89/11548

WO 90/01564

WO 91/02087

WO 92/15712

WO 95/11995

WO 95/12607

WO 96/21144

WO 97/10366

WO 97/31256

WO 98/12355

WO 98/14616

WO 98/20165

WO 98/30717

WO 98/30883

WO 98/44157

WO 00/55372

WO 00/66607

WO 01/32929

EP 235,726

EP 717,113

FR 2,650,840

GB 2,202,328

### Publications

Anderson and Young, 1985

Ardrey, 1992

Bains, 1992

Barinaga, 1991

Beltz et al., 1985

Chee et al., 1996

Connor et al., 1983

Drmanac et al., 1989

Effenhauser et al., 1994

Fan, 1997

Fodor et al., 1993

Frohman, 1990

Grant and Dervan, 1996

Gu et al., 1998

Guo et al., 1994

Hacia, 1996

Haff, 1997

Harrison et al., 1993

Hauser et al., 1998

Higuchi, 1992

Holland et al., 1991

Holmstrom, 1993

Hunkapiller, 1991

Jacobsen et al., 1994

Komher et al., 1989

Kornberg et al., 1992

Koster et al., 1987

Kozal et al., 1996

Kuppuswamy et al., 1991

Lai et al., 1998

Landegren et al., 1988

Lee, 1993

Manz et al., 1992

Marino, 1996

Meyer and Geider, 1979

Nanibhushan and Rabin, 1987

Newton et al., 1993

Nickerson et al., 1990

Nyreen et al., 1993

Ohara et al., 1989

Pastinen, 1996

Prezant et al., 1992

Ranki et al., 1983

Ross et al., 1997

Running et al., 1990

Saiki et al., 1986

Sambrook et al., 1989

Smith, 1990

Sokolov, 1990

Southern et al., 1992

Strezoska et al., 1991

Syv anen et al., 1990

Taboret al., 1989

Taillon-Miller et al., 1998

Ugozzoli et al., 1992

Wallace et al., 1979

Weiss, 1998

Williams, 1989

Woolley and Mathies, 1994

Wu et al., 1989

Zhao et al., 1998

We claim:

1. A method of amplifying a single nucleotide polymorphism (SNP) from a DNA sample, comprising:
a) obtaining the DNA sample comprising said single nucleotide polymorphism to be amplified;
b) generating at least one nick translate molecule from said DNA sample, wherein said nick translate molecule comprises said single nucleotide polymorphism; and
c) amplifying said nick translate molecule.

2. The method of claim 1, wherein said step of generating the nick translate molecule comprises:
a) attaching upstream adaptor molecules to ends of DNA sample molecules to provide a nick translation initiation site;
b) subjecting the DNA molecules to nick translation comprising DNA polymerization and 5′-3′ exonuclease activity to produce the nick translate molecules; and
c) attaching downstream adaptor molecules to the nick translate molecules to produce adaptor attached nick translate molecules.

3. A method of producing a library of SNP-containing DNA molecules, comprising:
a) obtaining a DNA sample comprising at least one SNP;
b) digesting DNA molecules of the DNA sample with a sequence-specific endonuclease;
c) attaching upstream adaptor molecules to ends of DNA molecules of the sample to provide a nick translation initiation site;
d) subjecting the DNA molecules to nick translation comprising DNA polymerization and 5′-3′ exonuclease activity to produce the nick translate molecules, wherein said nick translate molecules comprise said SNP;
e) attaching downstream adaptor molecules to the nick translate molecules to produce adaptor attached nick translate molecules; and
f) separating the SNP-containing nick translate molecules.

4. The method of claim 3, wherein said separating step is by size.

5. The method of claim 3, wherein said separating step is by hybridization.

6. The method of claim 3, wherein said separating step further comprises amplification of at least one said SNP-containing nick translate molecules.

7. The method of claim 6, wherein said amplification is by polymerase chain reaction.

8. A method of analyzing a SNP from a plurality of DNA samples, comprising:
a) obtaining said plurality of DNA samples, wherein at least one DNA sample comprises said SNP;
b) digesting DNA molecules of the DNA sample with a sequence-specific endonuclease;
c) attaching upstream adaptor molecules to ends of DNA molecules of the sample to provide a nick translation initiation site;
d) subjecting the DNA molecules to nick translation comprising DNA polymerization and 5′-3′ exonuclease activity to produce the nick translate molecules; wherein said nick translate molecules comprise said at least one SNP;
e) attaching downstream adaptor molecules to the nick translate molecules to produce adaptor attached nick translate molecules; and
f) separating the SNP-containing nick translate molecules.

9. The method of claim 8, wherein the upstream adaptors are nonidentical.

10. The method of claim 8, wherein said separating step is by size.

11. The method of claim 8, wherein said separating step is by hybridization.

12. The method of claim 8, wherein said separating step further comprises amplification of said SNP-containing nick translate molecules.

13. A method of isolating a specific SNP-containing nick translate molecule from a plurality of nick translate molecules, comprising:
a) obtaining a plurality of SNP-containing nick translate molecules;
b) ligating to an end of the SNP-containing nick translate molecules a first oligonucleotide to form a first oligonucleotide-nick translate molecule complex, wherein said first oligonucleotide comprises
i) nucleic acid sequence complementary to an adaptor end of said nick translate molecules;
ii) a double stranded region; wherein the double stranded region facilitates the formation of an adjacent hairpin or loop in the oligonucleotide;
iii) a free 3′ OH; and
iv) a 5′ phosphate;
c) attaching to said first oligonucleotide-nick translate molecule complex a second oligonucleotide to form a first oligonucleotide-nick translate molecule-second oligonucleotide-complex, wherein the second oligonucleotide comprises:
i) nucleic acid sequence adjacent to an adaptor end of said nick translate molecules;
ii) nucleic acid sequence nonidentical to a restriction endonuclease site used in generating the nick translate molecules; and
iii) an affinity tag;
d) isolating the nick translate molecule-first oligonucleotide-second oligonucleotide-complex from said plurality of nick translate molecules by said affinity tag.

14. The method of claim 13, wherein said attaching step further comprises ligation of said second oligonucleotide to said first oligonucleotide-nick translate molecule complex.

15. The method of claim 13, wherein said first oligonucleotide further comprises a labile base.

16. The method of claim 13, wherein said double stranded region of said first oligonucleotide is approximately six to eight bases.

17. The method of claim 13, wherein said double stranded region of said first oligonucleotide is at least about 4 bases.

18. The method of claim 13, wherein said double stranded region of said first oligonucleotide is no more than about 100 bases.

19. The method of claim 13, wherein said nucleic acid sequence in said second oligonucleotide which corresponds to the nucleic acid sequence adjacent to an adaptor end of said nick translate molecules is five nucleotides in length.

20. The method of claim 13, wherein the affinity tag of said second oligonucleotide is biotin.

21. A method of isolating a complementary nucleic acid molecule to a specific SNP-containing nick translate molecule, comprising:
a) obtaining a plurality of nick translate molecules;
b) introducing to said plurality an oligonucleotide comprising:
i) a nucleic acid sequence complementary to a specific region of said specific nick translate molecule;
ii) a nucleic acid sequence substantially nonidentical to a sequence in said specific nick translate molecule, wherein the nucleic acid sequence is 5′ to said sequence in i); and
iii) an affinity tag,
wherein the oligonucleotide hybridizes to the specific nick translate molecule;
c) extending the oligonucleotide by polymerization to form a complementary nucleic acid molecule for the specific nick translate molecule; and
d) isolating the extended complementary nucleic acid sequence molecule from the plurality of nick translate molecules.

22. The method of claim 21, wherein the method further comprises amplifying said complementary nucleic acid molecule.

23. The method of claim 22, wherein said amplification step is by polymerase chain reaction.

24. The method of claim 21, wherein the oligonucleotide further comprises a hairpin or loop structure.

25. A method of amplifying a nucleic acid sequence for SNP analysis, comprising:
a) generating a nick translate molecule comprising the nucleic acid sequence and comprising an upstream adaptor and a downstream adaptor;
b) performing polymerase chain reaction to amplify said nick translate molecule using a first oligonucleotide complementary to an adaptor sequence of said nick translate molecule and a second oligonucleotide complementary to a known nucleic acid sequence of said nick translate molecule.

26. The method of claim 25, wherein the step of generating said nick translate molecule comprises:
a) attaching said upstream adaptor molecule to ends of DNA molecules comprising said nucleic acid sequence for SNP analysis to provide a nick translation initiation site;
b) subjecting the DNA molecules to nick translation comprising DNA polymerization and 5′-3′ exonuclease activity to produce the nick translate molecules; and
c) attaching downstream adaptor molecules to the nick translate molecules to produce adaptor attached nick translate molecules.

27. A method of multiplex amplification of a plurality of nucleic acid sequences for SNP analysis, comprising:
a) generating a plurality of nick translate molecules comprising a nucleic acid sequence comprising said SNP, wherein each nick translate molecule comprises a first adaptor and a second adaptor;
b) introducing to said plurality of nick translate molecules a plurality of first oligonucleotides complementary to said first or second adaptor sequence of said nick translate molecules and a plurality of second oligonucleotides, wherein each second oligonucleotide is complementary to a known nucleic acid sequence in a nick translate molecule; and
c) amplifying the region in the nucleic acid sequence of said nick translate molecules between said first oligonucleotide and said second oligonucleotide by polymerase chain reaction.

28. A method of multiplex amplification of a plurality of nucleic acid sequences for SNP analysis, comprising:
a) generating a plurality of nick translate molecules each comprising a nucleic acid sequence comprising said SNP, wherein each nick translate molecule comprises a first adaptor and a second adaptor;
b) introducing to said plurality of nick translate molecules a plurality of first oligonucleotides complementary to said first adaptor sequence of said nick translate molecules and a plurality of second oligonucleotides, wherein the second oligonucleotide comprises
i) nucleic acid sequence complementary to said second adaptor; and
ii) multiple nucleotide bases at the 3 terminal end of said second oligonucleotide which are complementary to corresponding multiple nucleotide bases in the nucleic acid sequence of said nick translate molecule immediately adjacent to said second adaptor;
c) amplifying the region in the nucleic acid sequence of said nick translate molecules between said first oligonucleotide and said second oligonucleotide by polymerase chain reaction, whereby the amplification of the nucleic acid sequence occurs only under conditions wherein the second oligonucleotide anneals to said nick translate molecule at said multiple nucleotide bases immediately adjacent to the second adaptor.

29. The method of claim 28, wherein said multiple nucleotide bases comprise two bases.

30. The method of claim 28, wherein said multiple nucleotide bases comprise three bases.

31. A method of multiplex amplification of a nucleic acid sequence comprising a SNP of interest, wherein the nucleic acid sequence is adjacent to a known nucleic acid sequence, comprising:
a) obtaining a DNA sample;
b) processing said DNA sample to generate a library of nick translate molecules, wherein said nick translate molecules are separated into sublibraries of molecules that are complementary to specified positions within a region of the DNA, and wherein said sublibraries are partitioned into chambers of a solid support; and
c) amplifying by polymerase chain reaction within said chambers at least one nick translate molecule or fragment thereof using a primer from said known nucleic acid sequence.

32. The method of claim 31, wherein said DNA sample further comprises a genome.

33. The method of claim 31, wherein said solid support is a microwell plate.

34. A method of multiplex amplification of a nucleic acid sequence comprising a SNP of interest, wherein the nucleic acid sequence is adjacent to a known nucleic acid sequence, comprising:
a) obtaining a DNA sample;
b) processing said DNA sample to generate a library of nick translate molecules, wherein said nick translate molecules are in a pooled collection and wherein the nick translate molecules are comprised of sequences complementary to unknown positions within a region of the template DNA; and
c) amplifying by polymerase chain reaction within said pooled collection at least one nick translate molecule or fragment thereof using a primer from said known nucleic acid sequence.

35. The method of claim 34, wherein said pooled collection is in a single tube.

36. The method of claim 34, further comprising applying said amplified nick translate molecules to a DNA microarray, wherein hybridization of a nick translate molecule to the DNA microarray identifies said SNP.

37. A method of assaying a DNA sample for the presence of multiple specific SNPs, comprising:
a) generating a plurality of nick translate molecules from said DNA molecules of said sample, wherein said plurality of nick translate molecules comprise said multiple SNPs;
b) introducing to said nick translate molecules a plurality of oligonucleotides, wherein an oligonucleotide hybridizes adjacent to a specific SNP location and wherein the 3′ base of said oligonucleotide is variable;
c) extending by polymerization from said oligonucleotide, whereby extension only occurs if said variable 3′ base of said oligonucleotide is complementary to the corresponding nucleotide of said specific SNP; and
d) detecting said extended oligonucleotide.

38. The method of claim 37, wherein said detection step further comprises separation by size.

39. The method of claim 38, wherein said size detection is by capillary electrophoresis.

40. The method of claim 37, wherein said extended oligonucleotide is detected by detecting a label on the 3′ base of said oligonucleotide.

41. The method of claim 40, wherein said label is fluorescent.

42. The method of claim 37, wherein multiple specific SNPs are detected concomitantly, and wherein the labels for multiple nonidentical oligonucleotides in said plurality of oligonucleotides are distinguishable.

43. A method of assaying a DNA sample for the presence of multiple specific SNPs, comprising:
a) generating a plurality of nick translate molecules from said DNA molecules of said sample, wherein said plurality of nick translate molecules comprise said SNP;
b) introducing to said nick translate molecules a plurality of first oligonucleotides, wherein a first oligonucleotide hybridizes such that its 5′ end is adjacent to a specific SNP;
c) extending said first oligonucleotide by primer extension to form a plurality of nick translate molecule-first oligonucleotide extension product hybrids;
d) introducing to said plurality of hybrids a plurality of second oligonucleotides, wherein a second oligonucleotide hybridizes adjacent to the specific SNP and comprises a variable nucleotide 3′ end; and
e) ligating the 3′ end of said second oligonucleotide to the 5′ end of said first oligonucleotide extension product, whereby said ligation occurs only if said variable nucleotide is complementary to said SNP, to form a ligated molecule of said second oligonucleotide and said first oligonucleotide extension product; and
f) detecting said ligated molecule.

44. The method of claim 43, wherein said second oligonucleotide is fluorescently labeled.

45. The method of claim 43, wherein said plurality of second oligonucleotides are differentially fluorescently labeled.

46. The method of claim 43, wherein said detection step of said ligated molecule further comprises separation by size.

47. The method of claim 46, wherein said size separation is by capillary electrophoresis.

48. A method of analyzing at least one SNP from a plurality of individuals, comprising:
a) generating at least one specific nick translate molecule from DNA samples from each individual, wherein said specific nick translate molecule comprises the SNP; and
b) detecting said SNP.

49. The method of claim 48, wherein said detection step further comprises:
a) introducing to the nick translate molecule from the plurality of individuals a plurality of oligonucleotides, wherein said oligonucleotides hybridize adjacent to said SNP and wherein the 3′ base of said oligonucleotide is variable;
b) extending by polymerization from said oligonucleotide, whereby extension only occurs if said variable 3′ base of said oligonucleotide is complementary to the corresponding nucleotide of said SNP; and
c) detecting said extended oligonucleotide.

50. The method of claim 49, wherein said method further comprises separating said extended oligonucleotides by size.

51. The method of claim 50, wherein said size separation is by electrophoresis.

52. The method of claim 49, wherein said extended oligonucleotides are detected by fluorescent label.

53. The method of claim 48, wherein said detection step further comprises:
a) introducing to the nick translate molecules from the plurality of individuals a plurality of first oligonucleotides, wherein a first oligonucleotide hybridizes such that its 5′ end is adjacent to the SNP;
b) extending said first oligonucleotide by primer extension to form a plurality of nick translate molecule-first oligonucleotide extension product hybrids;
c) introducing to said plurality of hybrids a plurality of second oligonucleotides, wherein a second oligonucleotide hybridizes adjacent to the SNP and comprises a variable nucleotide 3′ end; and
d) ligating the 3′ end of said second oligonucleotide to the 5′ end of said first oligonucleotide extension product, whereby said ligation occurs only if said variable nucleotide is complementary to said SNP, to form a ligated molecule of said second oligonucleotide and said first oligonucleotide extension product; and
e) detecting said ligated molecule.

54. The method of claim 53, wherein said detection step further comprises separating said ligated molecules by size.

55. The method of claim 54, wherein said size separation is by electrophoresis.

56. The method of claim 54, wherein said extended oligonucleotides are detected by fluorescent label.

57. A method of analyzing at least one SNP from DNA samples from a plurality of individuals, comprising:
a) generating from each of said DNA samples a specific nick translate molecule comprising said SNP, wherein an adaptor on one end of said nick translate molecule comprises a unique nucleic acid sequence;
b) introducing to said nick translate molecules a two-part oligonucleotide, comprising:
i) a first part comprising nucleic acid sequence complementary to the unique nucleic acid sequence of said adaptor; and
ii) a second part comprising nucleic acid sequence complementary to nucleic acid sequence immediately 5′ to the SNP;
whereby said introduction results in the hybridization of said two parts of the oligonucleotide to the respective complementary sequences of said nick translate molecule and results in the formation of a loop in said nick translate molecule to bring said two parts in proximity of each other;
c) introducing to said two-part oligonucleotide differentially fluorescently labeled dideoxynucleotide triphosphates and DNA polymerase;
d) incorporating into the two-part oligonucleotide the fluorescently labeled dideoxynucleotide triphosphate which is complementary to said SNP; and
e) detecting said SNP.

58. The method of claim 57, wherein said SNP detection step further comprises hybridization of said fluorescently labeled dideoxynucleotide triphosphate-incorporated two-part oligonucleotide to a solid support, wherein the solid support comprises multiple positions, wherein each position comprises a unique adaptor sequence.

59. The method of claim 58, wherein said solid support is a chip.

60. A method of amplification of a genome comprising a SNP of interest, comprising:
a) obtaining the genome;
b) generating a plurality of nick translate molecules from said genome, wherein at least one nick translate molecule comprises the SNP of interest; and
c) amplifying the SNP-containing nick translate molecule.

61. The method of claim 60, further comprising detection of said SNP.

62. The method of claim 61, wherein said SNP is detected by microarray analysis, sequencing, hybridization, or a combination thereof.

63. The method of claim 60, wherein said generating of the nick translate molecules comprises:
a) attaching upstream adaptor molecules to ends of DNA molecules in the genome to provide a nick translation initiation site;
b) subjecting the DNA molecules to nick translation comprising DNA polymerization and 5′-3′ exonuclease activity to produce the nick translate molecules; and
c) attaching downstream adaptor molecules to the nick translate molecules to produce adaptor attached nick translate molecules.
